(19) United States
(12) Patent Application Publication
(10) Pub. No.: US 2014/0206678 A1
(43) Pub. Date: Jul. 24, 2014

(54) INHIBITORS OF MTOR KINASE AS ANTI -VIRAL AGENT

(71) Applicants: Thomas Shenk, Princeton, NJ (US); Ji-In Kim, Princeton, NJ (US)

(72) Inventors: Thomas Shenk, Princeton, NJ (US); Ji-In Kim, Princeton, NJ (US)

(73) Assignees: KADMON CORPORATION, LLC, New York, NY (US); THE TRUSTEES OF PRINCETON UNIVERSITY, Princeton, NJ (US)

(21) Appl. No.: 13/982,331

(22) PCT Filed: Jan. 27, 2012
(86) PCT No.: PCT/US12/23035

§ 371 Date: Mar. 12, 2014

Related U.S. Application Data
(60) Provisional application No. 61/436,970, filed on Jan. 27, 2011.

(51) Int. Cl. A61K 31/5377; A61K 31/5386; A61K 31/4745; A61K 45/06

(52) U.S. Cl. CPC A61K 31/5377; A61K 45/06; A61K 31/5386; A61K 31/4745
USPC 514/230.5; 514/234.2; 514/293

(57) ABSTRACT

The present invention provides methods for treating or preventing viral infections using modulators of host cell enzymes relating to mTOR. The invention also provides methods for treating or preventing viral infections using modulators of host cell enzymes relating to mTOR and modulators of the unfolded protein response.

### CROSS-REFERENCE TO RELATED APPLICATIONS

This application claims priority to U.S. Application No. 61/436,970, filed Jan. 27, 2011, which is incorporated herein by reference in its entirety.

### STATEMENT OF GOVERNMENT INTEREST

The invention was made with government support under Grant No. CA085786 awarded by the National Institutes of Health. The Government has certain rights in this invention.

### FIELD OF THE INVENTION

The present invention provides methods for treating or preventing viral infections using modulators of host cell enzymes relating to mTOR. The invention also provides methods for treating or preventing viral infections using modulators of host cell enzymes relating to mTOR and modulators of the unfolded protein response.

### BACKGROUND OF THE INVENTION

The mammalian target of rapamycin (mTOR) is a serine/threonine kinase that functions to regulate translation. mTOR exists in two complexes called mTOR complex 1 (mTORC1) and mTOR complex 2 (mTORC2). In addition to the mTOR catalytic subunit, mTORC1 contains additional proteins, including Raptor, mLST8, and PRAS40. mTORC2 contains mTOR and mLST8, but also contains the regulatory proteins Rictor, mSIN1, and PROTOR. In addition, mTORC1 and mTORC2 interact with DEPTOR, which inhibits their activities.

Rapamycin is an immunosuppressant used to prevent rejection in organ transplantation. Rapamycin and its analogs inhibit mTOR by binding to the FKBP-12 protein and mediating the formation of a complex with the FKBP-rapamycin binding (FKB) domain of mTOR. This interaction inhibits certain functions of mTORC1 such as S6K phosphorylation. However, there are other functions of mTORC1 that are resistant to rapamycin such as phosphorylation of 4EBP (eIF4E-binding protein). In addition, mTORC2 function is resistant to rapamycin inhibition because the FKBP-rapamycin complex does not interact with mTORC2.

There is a great unmet medical need for agents that more safely, effectively, and reliably treat viral infections, from HIV to the common cold. This includes a major need for better agents to treat human cytomegalovirus (where current agents suffer from significant toxicity and lack of efficacy), herpes simplex virus (where current agents are beneficial but provide incomplete relief), influenza A (where resistance to current agents is rampant), and hepatitis C virus (where many patients die from poor disease control). It further includes a major need for agents that work across a spectrum of viruses, facilitating their clinical use without necessarily requiring identification of the underlying pathogen.

### SUMMARY OF THE INVENTION

In a first aspect, the invention features a method of treating or preventing viral infection in a mammal, comprising administering to a mammalian subject in need thereof a therapeutically effective amount of a compound or prodrug thereof, or pharmaceutically acceptable salt or ester of said compound or prodrug, wherein the compound is an inhibitor of a rapamycin-resistant function of mTOR.

In another aspect, the invention features a pharmaceutical composition for treatment or prevention of a viral infection comprising a therapeutically effective amount of a composition comprising (i) a compound or prodrug thereof, or pharmaceutically acceptable salt of said compound or prodrug; and (ii) a pharmaceutically acceptable carrier, wherein the compound is an inhibitor of a rapamycin-resistant function of mTOR.

In another aspect, the invention features the use of a compound or prodrug thereof, or pharmaceutically acceptable salt of said compound or prodrug, wherein the compound is an inhibitor of a rapamycin-resistant function of mTOR, in the manufacture of a medicament for treatment or prevention of a viral infection.

In another aspect, the invention features a compound or prodrug thereof, or pharmaceutically acceptable salt or ester of said compound or prodrug for use in treating or preventing viral infection in a mammal, wherein the compound is an inhibitor of a rapamycin-resistant function of mTOR.

In one embodiment the inhibitor of a rapamycin-resistant function of mTOR is a compound of Formula VIII

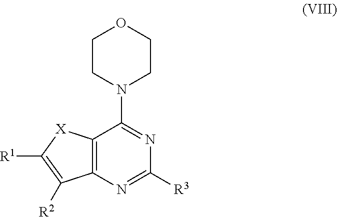

wherein:
X is O or S;
R¹ is selected from H, F, Cl, Br, I, CN, —CR¹⁴R¹⁵—NR¹⁶R¹⁷, —CR¹⁴R¹⁵—NHR¹⁰, —(CR¹⁴R¹⁵)ₜNR¹⁰R¹¹, —C(R¹⁴R¹⁵)ₙNR¹²C(═Y)R¹⁰, —(CR¹⁴R¹⁵)ₙNR¹²S(O)₂R¹⁰, —(CR¹⁴R¹⁵)ₘOR¹⁰, —(CR¹⁴R¹⁵)ₙS(O)₂R¹⁰, —(CR¹⁴R¹⁵)ₙS(O)₂NR¹⁰R¹¹, —C(OR¹⁰)R¹¹R¹⁴, —C(R¹⁴)═CR¹⁵R¹⁹, —C(═Y)R¹⁰, —C(═Y)OR¹⁰, —C(═Y)NR¹⁰R¹¹, —C(═Y)NR¹²OR¹⁰, —C(═O)NR¹²S(O)₂R¹⁰, —C(═O)NR¹²(CR¹⁴R¹⁵)ₘNR¹⁰R¹¹, —NO₂, —NHR¹², —NR¹²C(═Y)R¹¹, —NR¹²C(═Y)OR¹¹, —NR¹²C(═Y)NR¹⁰R¹¹, —NR¹²S(O)₂R¹⁰, —NR¹²SO₂NR¹⁰R¹¹, —S(O)₂R¹⁰, —S(O)₂NR¹⁰R¹¹, —SC(═Y)R¹⁰, —SC(═Y)OR¹⁰, C₂-C₁₂ alkyl, C₂-C₁₂ alkyl-R¹⁰, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl;
R² is selected from H, F, Cl, Br, I, CN, CF₃, —NO₂, —C(═Y)R¹⁰, —C(—Y)OR¹⁰, —C(═Y)NR¹⁰R¹¹, —(CR¹⁴R¹⁵)ₘNR¹⁰R¹¹, —(CR¹⁴R¹⁵)ₙOR¹⁰, —(CR¹⁴R¹⁵)ₜ—NR¹²C(═O)(CR¹⁴R¹⁵)NR¹⁰R¹¹, —NR¹²C(═Y)R¹⁰, —NR¹²C(═Y)OR¹⁰, —NR¹²C(═Y)NR¹⁰R¹¹, —NR¹²SO₂R¹⁰, OR¹⁰, —OC(═Y)R¹⁰, —OC(═Y)OR¹⁰, —OC(═Y)NR¹⁰R¹¹, —OS(O)₂(OR¹⁰), —OP(═Y)(OR¹⁰)(OR¹¹), —OP(OR¹⁰)(OR¹¹), SR¹⁰, —S(O)R¹⁰, —S(O)₂R¹⁰, —S(O)₂NR¹⁰R¹¹, —S(O)(OR¹⁰), —S(O)₂(OR¹⁰), —SC(═Y)R¹⁰, —SC(═Y)OR¹⁰, —SC(═Y)NR¹⁰R¹⁰, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, and C₁-C₂₀ heteroaryl;
R³ is a C₂-C₅ heterocyclyl, a C₂-C₅ heteroaryl, a fused bicyclic C₄-C₂₀ heterocyclyl or a fused bicyclic C₃-C₂₀ heteroaryl, each of which are unsubstituted or are optionally substituted;
R¹⁰, R¹¹ and R¹² are independently H, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl,
or R¹⁰ and R¹¹ together with the nitrogen to which they are attached optionally form a saturated, partially unsaturated or fully unsaturated C₃-C₂₀ heterocyclic ring optionally containing one or more additional ring atoms selected from N, O or S, wherein said heterocyclic ring is optionally substituted with one or more groups independently selected from oxo, (CH₂)ₘOR¹⁰NR¹⁰R¹¹, CF₃, F, Cl, Br, I, SO₂R¹⁰, C(═O)R¹⁰, NR¹²C(═Y)R¹¹, NR¹²S(O)₂R¹¹, C(═Y)NR¹⁰R¹¹, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl and C₁-C₂₀ heteroaryl;
R¹⁴ and R¹⁵ are independently selected from H, C₁-C₁₂ alkyl, or —(CH₂)ₙ-aryl,
or R¹⁴ and R¹⁵ together with the atoms to which they are attached form a saturated or partially unsaturated C₃-C₁₂ carbocyclic ring,
R¹⁶ and R¹⁷ are independently H, C₁-C₁₂ alkyl, C₂-C₈ alkenyl, C₂-C₈ alkynyl, C₃-C₁₂ carbocyclyl, or C₆-C₂₀ aryl,
R¹⁸ and R¹⁹ together with the carbon to which they are attached form a C₃-C₂₀ heterocyclic ring,
where said alkyl, alkenyl, alkynyl, carbocyclyl, heterocyclyl, aryl and heteroaryl are optionally substituted with one or more groups independently selected from F, Cl, Br, I, CN, CF₃, —NO₂, oxo, R¹⁰, —C(═Y)R¹⁰, —C(═Y)OR¹⁰, —C(═Y)NR¹⁰R¹¹, —(CR¹⁴R¹⁵)ₙNR¹⁰R¹¹, —(CR¹⁴R¹⁵)ₙOR¹⁰, —NR¹⁰R¹¹, —NR¹²C(═Y)R¹⁰, —NR¹²C(═Y)OR¹¹, —NR¹²C(═Y)NR¹⁰R¹¹, —NR¹²SO₂R¹⁰, ═NR¹², OR¹⁰, —OC(═Y)R¹⁰, —OC(═Y)OR¹⁰, —OC(═Y)NR¹⁰R¹¹, —OS(O)₂(OR¹⁰), —OP(═Y)(OR¹⁰)(OR¹¹), —OP(OR¹⁰)(OR¹¹), SR¹⁰, —S(O)R¹⁰, —S(O)₂R¹⁰, —S(O)₂NR¹⁰R¹¹, —S(O)(OR¹⁰), —S(O)₂(OR¹⁰), —SC(═Y)R¹⁰, —SC(═Y)OR¹⁰, —SC(═Y)NR¹⁰R¹¹, C₁-C₁₂ optionally substituted alkyl, C₂-C₈ optionally substituted alkenyl, C₂-C₈ optionally substituted alkynyl, C₃-C₁₂ optionally substituted carbocyclyl, C₂-C₂₀ optionally substituted heterocyclyl, C₆-C₂₀ optionally substituted aryl, C₁-C₂₀ optionally substituted heteroaryl, —(CR¹⁴R¹⁵)ₜ—NR¹²C(═O)(CR¹⁴R¹⁵)NR¹⁰R¹¹, and (CR¹⁴R¹⁵)ₜNR¹⁰R¹¹;
Y is O, S, or NR¹²;
m is 0, 1, 2, 3, 4, 5 or 6;
n is 1, 2, 3, 4, 5 or 6; and
t is 1, 2, 3, 4, 5 or 6, and
wherein the substituent groups that are alkyls, alkenyls, alkynyls, carbocyclyls, heterocyclyls, aryls, heteroaryls, fused bicyclic heterocyclyls, and fused bicyclic heteroaryls are optionally substituted with one or more substituents selected from the group consisting of F, Cl, Br, I, CN, CF₃, —NO₂, —NH₂, oxo, R¹⁰, —C(═Y)R¹⁰, —C(═Y)OR¹⁰, —C(═Y)NR¹⁰R¹¹, —(CR¹⁴R¹⁵)ₙNR¹⁰R¹¹, —(CR¹⁴R¹⁵)ₙOR¹⁰, —NR¹⁰R¹¹, —NR¹²C(═Y)R¹⁰, —NR¹²C(═Y)OR¹¹, —NR¹²C(═Y)NR¹⁰R¹¹, —NR¹²SO₂R¹⁰, ═NR¹², OR¹⁰, —OC(═Y)R¹⁰, —OC(═Y)OR¹⁰, —OC(═Y)NR¹⁰R¹¹, —OS(O)₂(OR¹⁰), —OP(═Y)(OR¹⁰)(OR¹¹), —OP(OR¹⁰)(OR¹¹), SR¹⁰, —S(O)R¹⁰, —S(O)₂R¹⁰, —S(O)₂NR¹⁰R¹¹, —S(O)(OR¹⁰), —S(O)₂(OR¹⁰), —SC(═Y)R¹⁰, —SC(═Y)OR¹⁰, —SC(═Y)NR¹⁰R¹¹, C₁-C₁₂ optionally substituted alkyl, C₂-C₈ optionally substituted alkenyl, C₂-C₈ optionally substituted alkynyl, C₃-C₁₂ optionally substituted carbocyclyl, C₂-C₂₀ optionally substituted heterocyclyl, C₆-C₂₀ optionally substituted aryl, C₁-C₂₀ optionally substituted heteroaryl, —(CR¹⁴R¹⁵)ₜ—NR¹²C(═O)(CR¹⁴R¹⁵)NR¹⁰R¹¹, and (CR¹⁴R¹⁵)ₜ—NR¹⁰R¹¹.

In one embodiment the compound of formula VIII is GNE-493. In one embodiment wherein the compound of formula VIII is GNE-0941.

In one embodiment the compound of Formula VIII is a specific inhibitor of mTOR. In one embodiment the compound of Formula VIII is an inhibitor mTORC1. In another embodiment the compound of Formula I is an inhibitor of mTORC2.

In one embodiment the inhibitor of a rapamycin-resistant function of mTOR is a compound of Formula IX

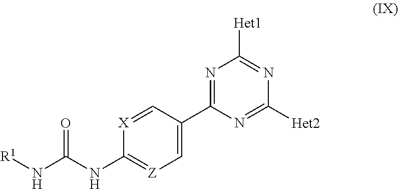

wherein,
Het1 and Het2 are independently selected from (i) a 5 to 7 membered heterocyclic group having 1-3 heteroatoms selected from O, N, and S, and (ii) a 6 to 10 membered bicyclic heterocyclic group having 1-3 heteroatoms selected from O, N, and S; wherein each of (i) or (ii) may by unsubstituted or substituted with 1 to 3 substituents selected from C₁-C₄ alkyl, NH₂, —O—R⁰, CN, and halo;
X and Z are independently selected from N and CH;
R¹ is selected from the group consisting of:
   (i) C₁-C₁₀ alkyl, C₂-C₁₀ alkenyl, C₂-C₁₀ alkynyl, and C₃ to C₁₀ cycloalkyl; each of which is unsubstituted or is substituted by 1 to 3 substituents selected from the group consisting of halo, —N(R⁰)₂, CN, NO₂, —O—(C₂-C₁₀ alkyl), aryl, heteroaryl, and heterocyclyl;
   (ii) aryl, heteroaryl and heterocyclyl; each of which is unsubstituted or is substituted by 1 to 3 substituents selected from the group consisting of halo, —N(R⁰)₂, CN, NO₂, C₁-C₆ alkyl, C₂-C₆ alkenyl, aryl, heteroaryl, heterocyclyl, —O—R⁰, —(C₁-C₁₀ alkyl)-OR⁰, —O—(C₁-C₁₀ alkyl)-OR⁰, —(C₁-C₁₀ alkyl)-N(R⁰)₂, —O—(C₂-C₁₀ alkyl)-N(R⁰)₂, —O—(C₂-C₁₀ alkyl)-C(═O)—N(R⁰)₂, —C(═O)—(C₂-C₁₀ alkyl)-N(R⁰)₂, —C(═O)N(R⁰)—(C₂-C₁₀ alkyl)-N(R⁰)₂, and —(C₁-C₆ alkyl)-aryl;
   (iii) -aryl-C(═O)-aryl, -aryl-C(═O)-heteroaryl, and -aryl-C(═O)-heterocyclyl; each of which is unsubstituted or is substituted by 1 to 3 substituents selected from the group consisting of halo, —N(R⁰)₂, CN, NO₂, C₁-C₆ alkyl, C₂-C₆ alkenyl, and —O—R⁰; and
   (iv) -aryl-aryl, -aryl-heteroaryl, and -aryl-heterocyclyl; each of which is unsubstituted or is substituted by 1 to 3 substituents selected from the group consisting of halo, —N(R⁰)₂, CN, NO₂, C₁-C₆ alkyl, C₂-C₆ alkenyl, —O—R⁰; and
each R⁰ is independently selected from H and C₁-C₆ alkyl; or alternatively two adjacent R⁰ attached to a nitrogen atom may be taken together to form a 5 or 6-membered heterocyclic ring having 0 to 2 additional ring heteroatoms selected from N, O, and S, and which may be unsubstituted or substituted by 1 to 3 substituents selected form the group consisting of C₁-C₄ alkyl, NH₂, —OH, CN, and halo.

In one embodiment the compound of Formula IX is Wyeth BMCL 20102644-13. In one embodiment the compound of Formula IX is Wyeth BMCL 20102648-27.

In one embodiment the compound of Formula IX is a specific inhibitor of mTOR. In one embodiment the compound of Formula IX is an inhibitor mTORC1. In one embodiment the compound of Formula II is an inhibitor of mTORC2.

In one embodiment the inhibitor of a rapamycin-resistant function of mTOR is a compound of Formula X:

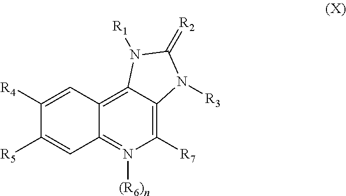

wherein
R₁ is selected from naphthyl or phenyl, which is unsubstituted or is substituted by one or two substituents independently selected from the group consisting of (i) halogen; (ii) lower alkyl unsubstituted or substituted by halogen, cyano, imidazolyl or triazolyl; (iii) cycloalkyl; (iv) amino substituted by one or two substituents independently selected from the group consisting of lower alkyl, lower alkyl sulfonyl, lower alkoxy and lower alkoxy lower alkylamino; (v) piperazinyl unsubstituted or substituted by one or two substituents independently selected from the group consisting of lower alkyl and lower alkyl sulfonyl; (vi) 2-oxo-pyrrolidinyl; (vii) lower alkoxy lower alkyl; (viii) imidazolyl; (ix) pyrazolyl; and (x) triazolyl;
R₂ is O or S;
R₃ is lower alkyl;
R₄ is selected from (i) pyridyl unsubstituted or substituted by halogen, cyano, lower alkyl, lower alkoxy or piperazinyl unsubstituted or substituted by lower alkyl; (ii) pyrimidinyl unsubstituted or substituted by lower alkoxy; (iii) quinolinyl unsubstituted or substituted by halogen; or (iv) quinoxalinyl;
R₅ is hydrogen or halogen;
n is 0 or 1;
R₆ is oxido; with the proviso that if n=1, the N-atom bearing the radical R₆ has a positive charge; and
R₇ is hydrogen or amino.

In one embodiment the compound of Formula X is NVP-BEZ235.

In one embodiment the compound of Formula X is a specific inhibitor of mTOR. In one embodiment the compound of Formula X is an inhibitor mTORC1. In another embodiment the compound of Formula X is an inhibitor of mTORC2.

In one embodiment, the compound is selected from AZD2014, CC-223 (TORKi), SB-2015, NVP-BGT-226, LY294002, GSK2126458, XL147, XL765, PF-04691502, P-2281, GDC-0980, ETP-45658, WJD008, PKI-179, PKI-402, PKI-587, OXA-01, Compound 401, ZSTK-474, PI-103, and Palomid-529.

In one embodiment the viral infection is by a herpesvirus. In one embodiment the viral infection is by a virus selected from herpes simplex virus (HSV) types 1 and 2, varicella-zoster virus, human cytomegalovirus (HCMV), Epstein-Barr virus (EBV), human herpesvirus 6 (variants A and B), human herpesvirus 7, human herpesvirus 8 (Kaposi's sarcoma—associated herpesvirus, KSHV), and cercopithecine herpesvirus 1 (B virus). In one embodiment the viral infection is by a virus selected from human cytomegalovirus and herpes simplex virus-1.

In one embodiment the invention further comprises administering to the mammalian subject an inhibitor of the unfolded protein response. In one embodiment the inhibitor of the unfolded protein response is 4-phenylbutyrate. In one embodiment the inhibitor of the unfolded protein response is tauroursodeoxycholic acid.

In one aspect the invention features the use of a first compound or prodrug thereof, or pharmaceutically acceptable salt of said first compound or prodrug, wherein the compound is an inhibitor of mTOR and a second compound or prodrug thereof, or pharmaceutically acceptable salt of said second compound or prodrug wherein the second compound is an inhibitor of the unfolded protein response in the manufacture of a medicament for treatment or prevention of a viral infection.

In one aspect, the invention features a method of treating or preventing a herpesvirus infection in a mammal, comprising administering to a mammalian subject in need thereof a therapeutically effective amount of a compound or prodrug thereof, or pharmaceutically acceptable salt or ester of said compound or prodrug, wherein the compound is an inhibitor of the unfolded protein response. In one embodiment, the compound is a chemical chaperone. In one embodiment, the compound is 4-phenylbutyrate. In one embodiment, the compound is tauroursodeoxycholic acid. In one embodiment the herpesvirus is selected from herpes simplex virus (HSV) types 1 and 2, varicella-zoster virus, human cytomegalovirus (HCMV), Epstein-Barr virus (EBV), human herpesvirus 6 (variants A and B), human herpesvirus 7, human herpesvirus 8 (Kaposi's sarcoma—associated herpes virus, KSHV), and cercopithecine herpesvirus 1 (B virus).

In another aspect, the invention features a pharmaceutical composition for treatment or prevention of a herpesvirus infection in a mammal comprising a therapeutically effective amount of a composition comprising (i) a compound or prodrug thereof, or pharmaceutically acceptable salt of said compound or prodrug; and (ii) a pharmaceutically acceptable carrier, wherein the compound is an inhibitor of the unfolded protein response. In one embodiment, the compound is a chemical chaperone. In one embodiment, the compound is 4-phenylbutyrate. In one embodiment, the compound is tauroursodeoxycholic acid. In one embodiment the herpesvirus is selected from herpes simplex virus (HSV) types 1 and 2, varicella-zoster virus, human cytomegalovirus (HCMV), Epstein-Barr virus (EBV), human herpesvirus 6 (variants A and B), human herpesvirus 7, human herpesvirus 8 (Kaposi's sarcoma—associated herpes virus, KSHV), and cercopithecine herpesvirus 1 (B virus).

In another aspect, the invention features the use of a compound or prodrug thereof, or pharmaceutically acceptable salt of said compound or prodrug, wherein the compound is an inhibitor of the unfolded protein response, in the manufacture of a medicament for treatment or prevention of a herpesvirus infection. In one embodiment, the compound is a chemical chaperone. In one embodiment, the compound is 4-phenylbutyrate. In one embodiment, the compound is tauroursodeoxycholic acid. In one embodiment the herpesvirus is selected from herpes simplex virus (HSV) types 1 and 2, varicella-zoster virus, human cytomegalovirus (HCMV), Epstein-Barr virus (EBV), human herpesvirus 6 (variants A and B), human herpesvirus 7, human herpesvirus 8 (Kaposi's sarcoma—associated herpes virus, KSHV), and cercopithecine herpesvirus 1 (B virus).

In another aspect, the invention features a compound or prodrug thereof, or pharmaceutically acceptable salt or ester of said compound or prodrug for use in treating or preventing a herpesvirus infection in a mammal, wherein the compound is an inhibitor of the unfolded protein response. In one embodiment, the compound is a chemical chaperone. In one embodiment, the compound is 4-phenylbutyrate. In one embodiment, the compound is tauroursodeoxycholic acid. In one embodiment the herpesvirus is selected from herpes simplex virus (HSV) types 1 and 2, varicella-zoster virus, human cytomegalovirus (HCMV), Epstein-Barr virus (EBV), human herpesvirus 6 (variants A and B), human herpesvirus 7, human herpesvirus 8 (Kaposi's sarcoma—associated herpes virus, KSHV), and cercopithecine herpesvirus 1 (B virus).

### DESCRIPTION OF THE FIGURES

FIG. 1. HCMV replication is inhibited by Torin1. Serum-starved confluent human fibroblasts were infected with HCMV at a multiplicity of 0.05 PFU/cell. Cell-free virus was quantified by a TCID₅₀ assay, and error bars represent the standard errors of the means from two independent experiments, each performed in duplicate. (A) Torin1 inhibits HCMV replication to a greater extent than does rapamycin. Immediately following viral adsorption, cells were treated with vehicle alone (N) (black bars) (dimethyl sulfoxide [DMSO]), rapamycin (T) (gray bars) (20 nM), or Torin1 (T) (white bars) (250 nM). Supernatants were harvested every other day and replaced with fresh medium containing the appropriate treatment, and virus in the supernatant was assayed on the indicated days. (B) Inhibition of HCMV replication is dose dependent and does not result from cellular toxicity. Infected fibroblasts were treated with various doses of Torin1. Medium with drug was replaced every other day, and virus in the supernatant was assayed on day 8 post infection (black bars). On day 8, a second set of cultures was washed twice, serum-free medium containing no drug was added to each well, and virus was assayed after an additional 8 days (16 days post infection) (white bars). (C) Torin1 is not toxic to uninfected human fibroblasts. The viability of fibroblasts treated with Torin1 (250 nM) was monitored over a time course of 10 days by a trypan blue exclusion assay.

FIG. 2. Torin1 does not affect HCMV entry into human fibroblasts. Serum-starved confluent fibroblasts were infected with HCMV at a multiplicity of 3 PFU/cell. (A) Torin1 does not block the entry of viral DNA. Serum-free confluent fibroblasts were pretreated with Torin1 (T) (250 nM) for 24 h prior to infection (Pre) or beginning immediately after adsorption at 1 hpi (Post). Control cultures received the vehicle in which Torin1 was dissolved (NT). At 2 hpi cells were harvested, and cell-associated viral DNA was quantified by real-time PCR analysis. Error bars represent the standard errors of the means from two independent experiments performed in duplicate. (B) Torin1 does not alter the accumulation of the HCMV IE1 protein. The level of IE1 was determined at 6 hpi by a Western blot assay using an IE1-specific monoclonal antibody. The image is representative of two independent experiments. (C) Torin1 does not alter the percentage of infected cells. The expression of a GFP marker gene present in the viral genome was monitored at 24 h after infection in the presence or absence of drug.

FIG. 3. Torin1 has little effect on the accumulation of an immediate-early protein and an early protein but inhibits the accumulation of HCMV DNA and a late protein. (A) Rapamycin-resistant mTOR activity is required for the accumulation of an some but not all HCMV proteins. Serum-starved confluent human fibroblasts were infected with HCMV at a multiplicity of 3 PFU/cell and then incubated with vehicle (N) (DMSO), rapamycin (R) (20 nM), or Torin1 (T) (250 nM) immediately following adsorption. Cells were harvested at the indicated times, and the accumulation of the indicated proteins was analyzed by Western blotting. (B) Torin1 inhibits HCMV DNA accumulation. Serum-starved confluent human fibroblasts were infected with HCMV at a multiplicity of 0.05 PFU/cell and incubated with vehicle, rapamycin, or Torin1 as described above (A). At the indicated times DNA was isolated, and viral DNA was quantified by qPCR. Equivalent amounts of DNA were analyzed for each sample, and the results are normalized to the level of actin DNA per sample. (C) The levels of the viral late transcript UL99 are inhibited by Torin1 treatment. Human fibroblasts were infected with HCMV at a multiplicity of 3 PFU/cell and treated with vehicle, rapamycin, or Torin1 as described above (A). At the indicated times the amount of UL99 RNA was determined by qPCR, and the results are normalized to the amount of actin RNA in each sample.

FIG. 4. Rapamycin-resistant mTOR activity is required for 4EBP1 phosphorylation and eIF4F complex integrity during HCMV infection. Serum-starved confluent human fibroblasts were infected with HCMV at a multiplicity of 3 PFU/cell. At 1 hpi, cultures were treated with the vehicle in which drugs were dissolved (N) (DMSO), rapamycin (R) (20 nM), or Torin1 (T) (250 nM). (A) At 48 hpi the phosphorylation status of mTORC1 targets was assessed by Western blot assay by using antibodies to phosphorylated targets (4EBP1-PT^{37/46} and rpS6-PS^{235/6}) and total proteins. Tubulin was assayed as a loading control. (B) Same as above (A) except that cells were harvested at the indicated times. (C and D) After mock infection (M) or infection with HCMV (WT) at a multiplicity of 3 PFU/cell, cultures were harvested at the indicated times. Equivalent amounts of protein from each sample were incubated with m⁷GTP-Sepharose, and the isolated protein complexes were analyzed by Western blotting using the indicated antibodies to the eIF4F complex and 4EBP1. In all cases the results are representative of at least two independent experiments. lys, lysate.

FIG. 5. Murine cytomegalovirus (MCMV) replication is inhibited by Torin1. (A) Torin1 but not rapamycin inhibits the production of MCMV progeny. Mouse embryo fibroblasts (MEFs) were infected with MCMV at a multiplicity of 0.05 PFU/cell and treated with vehicle (black bars) (DMSO), rapamycin (gray bars) (20 nM), or Torin1 (white bars) (250 nM). Fresh serum-free medium containing drugs was added every other day. At the indicated times, cell-free supernatants were harvested, and the amount of virus in the supernatant was quantified by the TCID₅₀ method. Error bars represent the standard errors of the means form two independent experiments performed in duplicate. (B) MEFs were infected with MCMV at a multiplicity of 3 PFU/cell and treated with vehicle (N), rapamycin (R), or Torin1 (T) as described above (A) or were treated with LY294002 (LY) (20 μM). At 48 hpi the phosphorylation state of the indicated mTORC1 targets was analyzed by a Western blot assay by using antibodies to phosphorylated targets (4EBP1-PT^{37/46} and rpS6-PS^{235/6}) and total proteins. The results are representative of three independent experiments.

FIG. 6. mTORC2 and its target, Akt, are not the source of rapamycin-resistant mTOR activity. (A) MCMV growth is inhibited by Torin1 in Rictor-null MEFs. Confluent serum-starved cells were infected with MCMV at a multiplicity of 0.05 PFU/cell, and vehicle (black bars) (DMSO), rapamycin (gray bars) (20 nM), or Torin1 (white bars) (250 nM) was added at 1 hpi. At 6 days post infection the amount of MCMV in cell-free supernatants was determined by the TCID₅₀ method. (B) Torin1 blocks 4EBP1 phosphorylation in Rictor-null MEFs. MEFs were mock infected (M) or infected with MCMV (WT) at a multiplicity of 3 PFU/cell and treated with vehicle (N), rapamycin (R), or Torin1 (T) as described above (A). At 48 hpi, the phosphorylation state of mTORC1 targets was assessed by Western blotting using antibodies specific for the indicated proteins. (C) Confirmation of the genotype of Rictor-null MEFs. Total DNA was isolated from wild-type and Rictor-null MEFs, and the genotype was confirmed by use of PCR. (D) Same as above (A) except that Akt1- and Akt2-null MEFs were used. (E) Same as above (B) except that Akt1- and Akt2-null MEFs were used. For B and E, the error bars represent the standard errors of the means from at least two independent experiments, each performed in duplicate. For C and E, tubulin was assayed as a loading control. (F) Akt is not expressed in Akt1- and Akt2-null MEFs. Protein from wild-type or mutant MEFs was analyzed by Western blotting by use of an antibody specific for Akt.

FIG. 7. Deletion of the mTORC1 target 4EBP1 rescues replication of MCMV in the presence of Torin1. (A) MCMV growth is not inhibited by Torin1 in 4EBP1-null MEFs. Confluent serum-starved cells were infected with MCMV at a multiplicity of 0.05 PFU/cell, and vehicle (black bars) (DMSO), rapamycin (gray bars) (20 nM), or Torin1 (white bars) (250 nM) was added at 1 hpi. At 6 days post infection the amount of MCMV in cell-free supernatants was determined by the TCID₅₀ method. The error bars represent the standard errors of the means from three independent experiments, each performed in duplicate. (B) Torin1 does not exclude eIF4G or eIF4A from the cap-binding complex in 4EBP1-null MEFs. Cells were infected with MCMV at a multiplicity of 3 PFU/cell and treated with vehicle (N), rapamycin (R), or Torin1 (T) as described above (A). At 48 hpi equal amounts of protein from cell lysates were incubated with m⁷G-Sepharose. The presence of eIF4F complex components bound by the cap analog was determined by Western blotting. The results are representative of two independent experiments.

FIG. 8. Rapamycin-resistant mTOR activity is required for lytic replication by representative alpha- and gamma-herpesviruses. (A) Confluent serum-starved MEFs were infected at a multiplicity of 0.05 PFU/cell with HSV-1 or γHV68. The amount of virus in cell-free supernatants was determined by the TCID₅₀ method at 72 hpi for HSV-1 (left) and at 6 days post infection for γHV68 (right). Black bars represent vehicle-treated samples (N) (DMSO), gray bars represent rapamycin-treated samples (R) (20 nM), and white bars represent Torin1-treated samples (T) (250 nM). The error bars represent the standard errors of the means from at least two independent experiments. (B) Confluent MEF monolayers were infected with HSV-1 at a multiplicity of 3 PFU/cell. Infected cell lysates were harvested at 8 hpi, and equal amounts of protein were analyzed by Western blotting. (C) WT or 4EBP1-null MEFs were infected with HSV-1 at a multiplicity of 0.05 PFU/cell. The amount of cell-free virus present in the supernatant at 72 hpi was quantified by the TCID₅₀ method. The error bars represent the standard errors of the means from two independent experiments.

FIG. 9. Inhibition of HCMV yield by treatment of human fibroblasts with siRNA directed against the mTOR kinase. MRC5 fibroblasts (ATCC #CCL-171) at passage 23-24 were plated at a density of 7500 cells/well in DMEM (Sigma-Aldrich product #D5756, St. Louis, Mo.) supplemented 10% FBS (GIBCO) in 96-well plastic tissue culture dishes. Cells were grown to ˜70% confluence and then transfected with 1 nmol siRNA targeting GFP mRNA (non-specific), the viral IE2 mRNA, or mTOR kinase using Oligofectamine (Invitrogen, Carlsbad, Calif.) per manufacturer's instructions. IE2 siRNA sequence: 5′-AAACGCAUCUCCGAGUUGGAC-3′ (SEQ ID NO:1); GFP siRNA sequence: 5′-GCAAGCUGACCCUGAAGUUCAU-3′ (SEQ ID NO:2); mTOR kinase (FRAP1_{—}2) siRNA sequence: 5′-GAGUUACAGUCGGGCAUAU-3′ (SEQ ID NO:3). All siRNAs were obtained from Sigma-Aldrich. 4 h post-transfection, medium was supplemented with FBS to 10% final concentration. 28 h post-transfection, culture supernatants were removed and replaced with 100 μl DMEM/10% FBS containing HCMV strain AD169 at a concentration of 0.1 pfu/cell. Infection proceeded for 96 h, at which time culture supernatants were harvested and used to infect a fresh plate of ˜90% confluent MRC5 cells in 96-well format. 24 h post-infection of this reporter plate, the samples were fixed with chilled methanol at −20° for 15 min and processed for immunofluorescence to quantify infectivity. Results are presented as “robust Z score”, which correlates with standard deviations from mean value for infectivity generated in the absence of siRNA treatment. Thus, the mTOR kinase-specific siRNA reduced the yield of infectious HCMV by a factor of >2 standard deviations, a highly significant effect.

FIG. 10. 4-PBA inhibits HCMV replication in a dose-dependent manner. Human fibroblasts were infected with HCMV strain AD169 at a multiplicity of 0.1 pfu/cell and maintained in medium containing 10% fetal calf serum and the indicated amount of drug. The medium with drug was replaced every other day. Cell-free and cell-associated virus was collected on day 8 post infection and titered by the TCID₅₀ method. Data represent the log mean titer of duplicate samples.

FIG. 11. 4-PBA is not toxic to uninfected or infected confluent human fibroblasts. (A) Fibroblasts were maintained in medium containing the indicated concentrations of 4-PBA for 8 days. The medium was replaced every other day throughout the time course. At the end of the treatment period, cell viability was measured by the trypan blue exclusion assay. Date points represent the mean of duplicate wells. (B) Fibroblasts were infected with HCMV at a multiplicity of 0.1 pfu/cell. Cells were fed every other day with fresh medium containing the indicated concentration of 4-PBA. At eight days post infection, cells were washed once with media, and then media lacking drug was added. Eight days later (16 days post infection) cell free virus in the supernatant was quantitated by the TCID₅₀ method. Date points represent the mean of duplicate wells.

FIG. 12. 4-PBA cooperates with mTOR inhibitors to interfere with HCMV replication in a dose-dependent manner. Human fibroblasts were infected with HCMV strain AD169 at a multiplicity of 0.1 pfu/cell and maintained in medium containing 10% fetal calf serum and the indicated drug(s). Drugs were used at the following concentrations: 4-PBA, 1 mM; Torin1, 250 nM; rapamycin, 20 nM. The medium with drug(s) was replaced every other day. Cell-free and cell-associated virus was collected on days 0, 4, 8 and 12 post infection, and titered by the TCID₅₀ method. Data represent the log mean titer of duplicate samples.

FIG. 13. Dose-dependent inhibition of human cytomegalovirus replication by mTOR inhibitors. About 95% confluent MRC5 human fibroblasts were infected with HCMV at a multiplicity of 0.5 pfu/cell and 2 hours after infection, the medium of the cells was replaced with fresh medium containing the indicated concentrations of BEZ235 (top panel, right), INK128 (top panel, left), OSI-027 (bottom panel, right), Wyeth—Compound 27 (Wyeth-BMCL20102648-27) (bottom panel, left). The yield of HCMV was determined at 96 hours post-infection and represented as a percentage of virus yield in untreated cells. Ganciclovir (a marketed drug for HCMV) was used as comparator for the efficacy of tested compounds in the assay. The effects of the compounds on the viability of uninfected MRC5 cells at 96 hours of treatment were assessed by using the Toxilight bioassay kit (Lonza).

### DETAILED DESCRIPTION

Viral replication requires energy and macromolecular precursors derived from the metabolic network of the host cell. Using an integrated approach to profiling metabolic flux, the inventors discovered alterations of certain metabolite concentrations and fluxes in response to viral infection. Details of the profiling methods are described in PCT/US2008/006959, which is incorporated by reference in its entirety. Using this approach, certain enzymes in the various metabolic pathways, especially those which serve as key “switches,” have been discovered to be useful targets for intervention; i.e., as targets for redirecting the metabolic flux to disadvantage viral replication and restore normal metabolic flux profiles, thus serving as targets for antiviral therapies. Enzymes involved in initial steps in a metabolic pathway are potential enzyme targets. In addition, enzymes that catalyze “irreversible” reactions or committed steps in metabolic pathways can be advantageously used as enzyme targets for antiviral therapy.

The subsections below describe in more detail the antiviral compounds and target enzymes of the invention, screening assays for identifying and characterizing new antiviral compounds, and methods for their use as antiviral therapeutics to treat and prevent viral infections. The compounds of the invention include inhibitors of mTOR activity and inhibitors of the unfolded protein response, which can be used alone or in combination to treat or prevent viral infection.

1. Modulators of mTOR

In one embodiment, the present invention provides a method of treating or preventing a viral infection in a mammal, comprising administering to a subject in need thereof a therapeutically effective amount of a compound or a relative, analogue, or derivative thereof, wherein the compound is an inhibitor of a rapamycin-resistant function of mTOR. An inhibitor of mTOR can inhibit mTORC1, mTORC2, or both mTORC1 and mTORC2. In one embodiment the compound is a specific inhibitor of mTOR. In one embodiment the compound is an inhibitor of mTOR and other kinases, including for example, PI3K.

Rapamycin and its analogs bind to the FKBP-12 protein and mediate the formation of a complex with the FKBP-Rapamycin Binding (FKB) domain of mTOR. This interaction inhibits certain functions of mTORC1 such as S6K phosphorylation. However, there are other functions of mTORC1 that are resistant to rapamycin such as phosphorylation of 4EBP (eIF4E-binding protein). In addition, mTORC2 function is resistant to rapamycin inhibition because the FKBP-Rapamycin complex does not interact with mTORC2. Thus, rapamycin-resistant functions of mTOR exist through mTORC1 and/or mTORC2.

1.1 Small Molecule Inhibitors

Compounds that inhibit rapamycin-resistant functions of mTOR include mTOR kinase domain inhibitors. Such compounds can, for example, selectively bind to the ATP binding site of the mTOR kinase domain. mTOR kinase inhibitors can be selective for mTOR showing >2, >5, >10, >20, >50, or >100 fold selectivity for the inhibition of mTOR over one or more kinases in Table 1 as measured by comparing, for example, the IC₅₀ values. In a preferred embodiment, the mTOR kinase inhibitor has >2, >5, >10, >20, >50, or >100 fold selectivity as compared to PI3K. In one embodiment, the compound that is an inhibitor of mTOR kinase also inhibits other kinases including, for example, PI3K.

Compounds of the invention include small molecules. As used herein, the terms “chemical agent” and “small molecule” are used interchangeably, and both terms refer to substances that have a molecular weight up to about 4000 atomic mass units (Daltons), preferably up to about 2000 Daltons, and more preferably up to about 1000 Daltons. Unless otherwise stated herein, the term “small molecule” as used herein refers exclusively to chemical agents, and does not refer to biological agents. As used herein, “biological agents” are molecules which include proteins, polypeptides, and nucleic acids, and have molecular weights equal to or greater than about 2000 atomic mass units (Daltons). Compounds of the invention include salts, esters, and other pharmaceutically acceptable forms of such compounds.

WO2010/044885, which is incorporated by reference in its entirety, describes small molecule modulators of mTOR. Described in this publication are pyridinonequinoline compounds of Formula I:

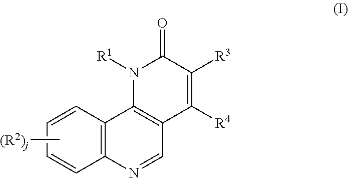

wherein R¹ is an optionally substituted group selected from the group consisting of 6-10-membered aryl; C₇₋₁₅ arylalkyl; C₆₋₁₅ heteroarylalkyl; C₁₋₁₂ heteroaliphatic; C₁₋₁₂ aliphatic; 5-10-membered heteroaryl having 1-4 heteroatoms independently selected from the group consisting of nitrogen, oxygen, and sulfur; and 4-7-membered heterocyclic having 1-2 heteroatoms independently selected from the group consisting of nitrogen, oxygen, and sulfur;

each occurrence of R² is independently halogen, —NR₂—OR, —SR, or an optionally substituted group selected from the group consisting Of C₁₋₁₂ acyl; 6-10-membered aryl; C₇₋₁₅ arylalkyl; C₆₋₁₅ heteroarylalkyl; C₁₋₁₂ heteroaliphatic; C₁₋₁₂ aliphatic; 5-10-membered heteroaryl having 1-4 heteroatoms independently selected from the group consisting of nitrogen, oxygen, and sulfur; and 4-7-membered heterocyclic having 1-2 heteroatoms independently selected from the group consisting of nitrogen, oxygen, and sulfur; j is an integer from 1 to 4, inclusive;

R³ and R⁴ are independently hydrogen, hydroxyl, alkoxy, halogen, or optionally substituted C₁₋₆ aliphatic, with the proviso that R³ and R⁴ are not taken together to form a ring; and each R is independently hydrogen, an optionally substituted group selected from the group consisting of C₁₋₁₂ acyl; 6-10-membered aryl; C₇₋₁₅ arylalkyl; C₆₋₁₅ heteroarylalkyl; C₁₋₁₂ aliphatic; 5-10-membered heteroaryl having 1-4 heteroatoms independently selected from the group consisting of nitrogen, oxygen, and sulfur; 4-7-membered heterocyclic having 1-2 heteroatoms independently selected from the group consisting of nitrogen, oxygen, and sulfur; and C₁₋₁₂ heteroaliphatic having 1-2 heteroatoms independently selected from the group consisting of nitrogen, oxygen, and sulfur; or

two R on the same nitrogen atom are taken with the nitrogen to form a 4-7-membered heterocyclic ring having 1-2 heteroatoms independently selected from the group consisting of nitrogen, oxygen, and sulfur.

Inhibitors of a rapamycin-resistant function of mTOR include the following:

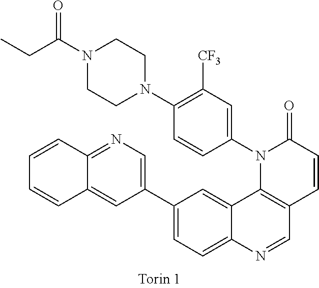 [SMILES: CCC(=O)N1CCN(c2ccc(-n3c(=O)ccc4cnc5ccc(-c6cnc7ccccc7c6)cc5c43)cc2C(F)(F)F)CC1]

is a pyridinonequinoline compound that is an ATP-competitive inhibitor of mTORC1 and mTORC2 with an IC₅₀ of about 2-10 nM. Torin1 is exemplified herein as an antiviral agent with activity against herpesvirus.

US 2009/0099174, which is incorporated by reference in its entirety, describes selective mTOR inhibitors. Described mTOR inhibitors include compounds of Formula II:

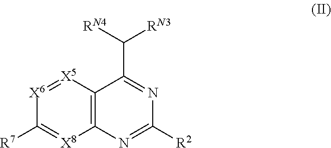

wherein

one or two of X⁵, X⁶ and X⁸ is N, and the others are CH;

R⁷ is selected from halo, OR^{O1}, SR^{S1}, NR^{N1}R^{N2}, NR^{N7a}C(═O)R^{C1}, NR^{N7b}SO₂R^{S2a}, an optionally substituted C₅₋₂₀ heteroaryl group, or an optionally substituted C₅₋₂₀ aryl group, where R^{O1} and R^{S1} are selected from H, an optionally substituted C₅₋₂₀ aryl group, an optionally substituted C₅₋₂₀ heteroaryl group, or an optionally substituted C₁₋₇ alkyl group; R^{N1} and R^{N2} are independently selected from H, an optionally substituted C₁₋₇ alkyl group, an optionally substituted C₅₋₂₀ heteroaryl group, an optionally substituted C₅₋₂₀ aryl group or R^{N1} and R^{N2} together with the nitrogen to which they are bound form a heterocyclic ring containing between 3 and 8 ring atoms; R^{C1} is selected from H, an optionally substituted C₅₋₂₀ aryl group, an optionally substituted C₅₋₂₀ heteroaryl group, an optionally substituted C₁₋₇ alkyl group or NR^{N8}R^{N9}, where R^{N8} and R^{N9} are independently selected from H, an optionally substituted C₁₋₇ alkyl group, an optionally substituted C₅₋₂₀ heteroaryl an optionally substituted C₅₋₂₀ aryl group or R^{N8} and R^{N9} together with the nitrogen to which they are bound form a heterocyclic ring containing between 3 and 8 ring atoms; R^{S2a} is selected from H, an optionally substituted C₅₋₂₀ aryl group, an optionally substituted C₅₋₂₀ heteroaryl group, or an optionally substituted C₁₋₇ alkyl group; R^{N7a} and R^{N7b} are selected from H and a C₁₋₄ alkyl group;

R^{N3} and R^{N4}, together with the nitrogen to which they are bound, form a heterocyclic ring containing between 3 and 8 ring atoms;

R² is selected from H, halo, OR^{O2}, SR^{S2b}, NR^{N5}R^{N6}, an optionally substituted C₅₋₂₀ heteroaryl group, and an optionally substituted C₅₋₂₀ aryl group, wherein R^{O2} and R^{S2b} are selected from H, an optionally substituted C₅₋₂₀ aryl group, an optionally substituted C₅₋₂₀ heteroaryl group, or an optionally substituted C₁₋₇ alkyl group; R^{N5} and R^{N6} are independently selected from H, an optionally substituted C₁₋₇ alkyl group, an optionally substituted C₅₋₂₀ heteroaryl group, and an optionally substituted C₅₋₂₀ aryl group, or R^{N5} and R^{N6} together with the nitrogen to which they are bound form a heterocyclic ring containing between 3 and 8 ring atoms.

The compound, Ku-0063794, is a selective inhibitor of mTOR and has the chemical structure:

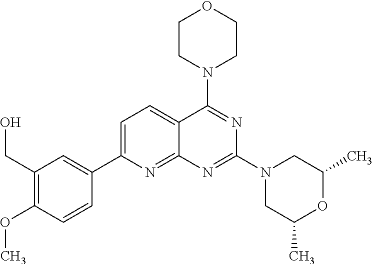 [SMILES: COc1ccc(-c2ccc3c(N4CCOCC4)nc(N4C[C@@H](C)O[C@@H](C)C4)nc3n2)cc1CO]

Ku-0063794 inhibits mTOR with an IC₅₀ of 10 nM and is selective with regard to PI3 kinases (P110α isoform IC₅₀ of 10 μM) (Garcia-Martinez et al. Biochem. J. 421:29-42).

WO2010/006072, which is incorporated by reference in its entirety describes selective mTOR inhibitors of Formula III or Formula IV:

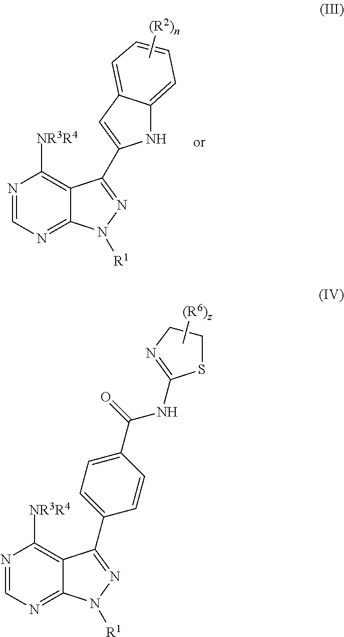

wherein, n is an integer from 1 to 5; z is an integer from 1 to 2;
R¹, R³, and R⁴ are independently hydrogen, halogen, —CN, —CF₃, —OH, —NH₂, —SO₂, —COOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R⁶ are independently hydrogen, halogen, —CN, —CF₃, —OR⁵, —NH₂, —SO₂, —COOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R⁵ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The compound, PP30, is one such compound of Formula IV, and has the following chemical structure:

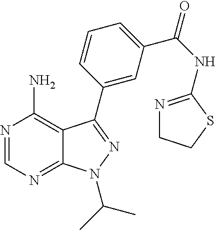 [SMILES: CC(C)n1nc(-c2cccc(C(=O)NC3=NCCS3)c2)c2c(N)ncnc21]

PP30 inhibits mTOR with an IC₅₀ of 80 nM and is selective with regard to PI3 kinases (P110α isoform IC₅₀ of 3 μM).

The compound, PP242, is one such compound of Formula III, and has the following chemical structure:

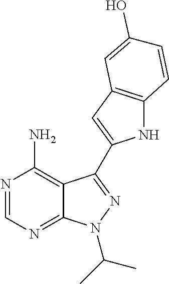 [SMILES: CC(C)n1nc(-c2cc3cc(O)ccc3[nH]2)c2c(N)ncnc21]

PP242 inhibits mTOR with an IC₅₀ of 8 nM and is selective with regard to PI3 kinases (P110α isoform IC₅₀ of 2 μM).

The invention further provides selective mTOR inhibitor of Formula V and VI as well as dual mTOR/PI3K inhibitors of Formula V, VI and VII:

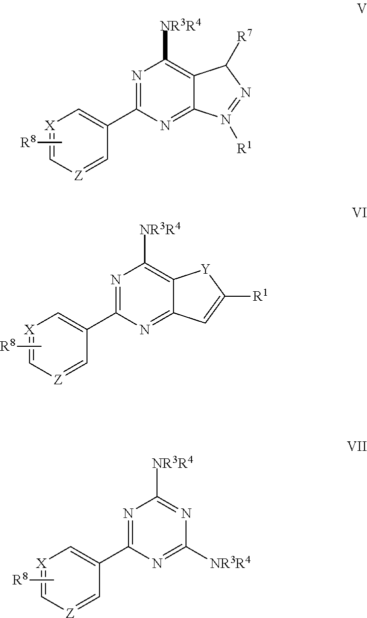

wherein
R¹, R³, R⁴, and R⁷ are independently hydrogen, halogen, —CN, —CF₃, —OH, —NH₂, —SO₂, —COOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
alternatively, R³ and R⁴ may be taken together with the nitrogen atom to which they are attached to form (i) a 5 to 7-membered heterocyclic group having 0 to 2 additional ring heteroatoms selected from N, O, and S, or (ii) a 6 to 9-membered hetero bicyclic group having 0 to 3 additional ring heteroatoms selected from N, O, and S; each of (i) and (ii) may be unsubstituted or may be substituted with 1 to 3 substituents selected from the group consisting of halogen, —CN, —CF₃, —OH, —NH₂, —SO₂, —COOH, substituted or unsubstituted alkyl.
R⁸ is —OH, —NH₂, substituted or unsubstituted urea; X and Z are independently N, or CH; and

### Y is S or NR⁷.

WYE-125132 and Wyeth-BMCL20096830-27 are compounds of Formula V and have the following chemical structures:

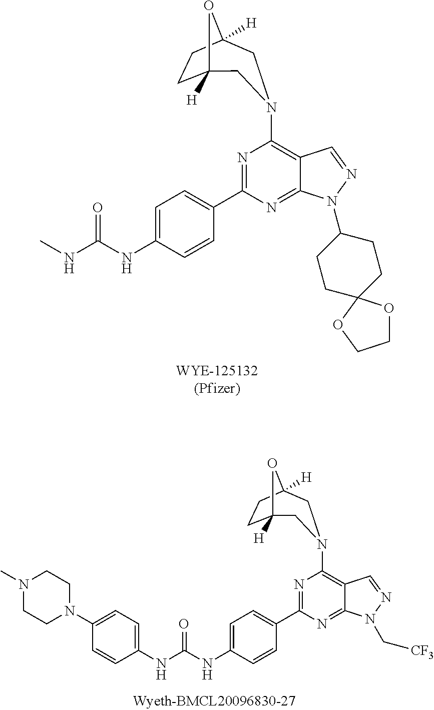 [SMILES: CNC(=O)Nc1ccc(-c2nc(N3C[C@H]4CC[C@@H](C3)O4)c3cnn(C4CCC5(CC4)OCCO5)c3n2)cc1 ; CN1CCN(c2ccc(NC(=O)Nc3ccc(-c4nc(N5C[C@H]6CC[C@@H](C5)O6)c5cnn(CC(F)(F)F)c5n4)cc3)cc2)CC1]

GNE-493 and GNE-0941 are compounds of Formula VI and have the following chemical structures:

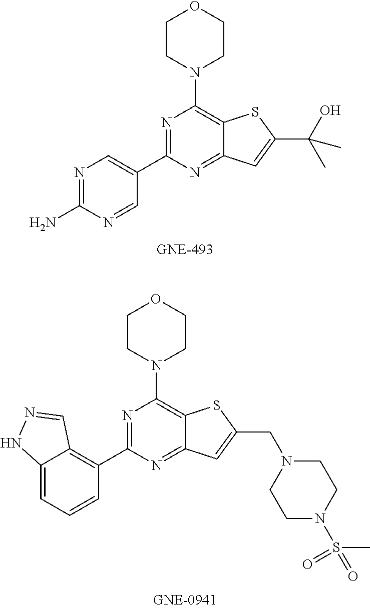 [SMILES: CC(C)(O)c1cc2nc(-c3cnc(N)nc3)nc(N3CCOCC3)c2s1 ; CS(=O)(=O)N1CCN(Cc2cc3nc(-c4cccc5[nH]ncc45)nc(N4CCOCC4)c3s2)CC1]

Wyeth BMCL 20102644-13 and Wyeth BMCL 20102648-27 are compounds of Formula VII and have the following chemical structures:

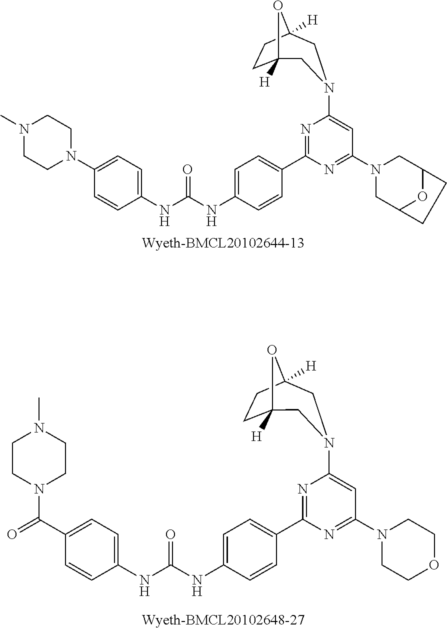 [SMILES: CN1CCN(c2ccc(NC(=O)Nc3ccc(-c4nc(N5CC6CCC(C5)O6)cc(N5C[C@H]6CC[C@@H](C5)O6)n4)cc3)cc2)CC1 ; CN1CCN(C(=O)c2ccc(NC(=O)Nc3ccc(-c4nc(N5CCOCC5)cc(N5C[C@H]6CC[C@@H](C5)O6)n4)cc3)cc2)CC1]

The present invention further provides compounds of Formula (VIII):

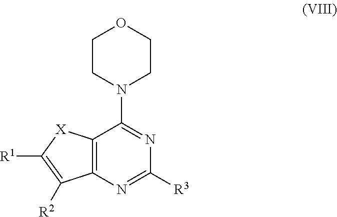

and pharmaceutically acceptable salts thereof, wherein:

### X is O or S;

R¹ is selected from H, F, Cl, Br, I, CN, —CR¹⁴R¹⁵—NR¹⁶R¹⁷, —CR¹⁴R¹⁵—NHR¹⁰, —(CR¹⁴R¹⁵)ₜNR¹⁰R¹¹, —C(R¹⁴R¹⁵)ₙNR¹²C(═Y)R¹⁰, —(CR¹⁴R¹⁵)NR¹²S(O)₂R¹⁰, —(CR¹⁴R¹⁵)ₘOR¹⁰, —(CR¹⁴R¹⁵)ₙS(O)₂R¹⁰, —(CR¹⁴R¹⁵)ₙS(O)₂NR¹⁰R¹¹, —C(OR¹⁰)R¹¹R¹⁴, —C(R¹⁴)═CR¹⁸R¹⁹, —C(═Y)R¹⁰, —C(═Y)OR¹⁰, —C(═Y)NR¹⁰R¹¹, —C(═Y)NR¹²OR¹⁰, —C(═O)NR¹²S(O)₂R¹⁰, —C(═O)NR¹²(CR¹⁴R¹⁵)ₘNR¹⁰R¹¹, —NO₂, —NHR¹², —NR¹²C(═Y)R¹¹, —NR¹²C(═Y)OR¹¹, —NR¹²C(═Y)NR¹⁰R¹¹, —NR¹²S(O)₂R¹⁰, —NR¹²SO₂NR¹⁰R¹¹, —S(O)₂R¹⁰, —S(O)₂NR¹⁰R¹¹, —SC(═Y)R¹⁰, —SC(═Y)OR¹⁰, C₂-C₁₂ alkyl, C₂-C₁₂ alkyl-R¹⁰, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl;
R² is selected from H, F, Cl, Br, I, CN, CF₃, —NO₂, —C(═Y)R¹⁰, —C(—Y)OR¹⁰, —C(═Y)NR¹⁰R¹¹, —(CR¹⁴R¹⁵)ₘNR¹⁰R¹¹, —(CR¹⁴R¹⁵)ₙOR¹⁰, —(CR¹⁴R¹⁵)ₜ—NR¹²C(═O)(CR¹⁴R¹⁵)NR¹⁰R¹¹, —NR¹²C(═Y)R¹⁰, —NR¹²C(═Y)OR¹⁰, —NR¹²C(═Y)NR¹⁰R¹¹, —NR¹²SO²R¹⁰, OR¹⁰, —OC(═Y)R¹⁰, —OC(═Y)OR¹⁰, —OC(═Y)NR¹⁰R¹¹, —OS(O)₂(OR¹⁰), —OP(═Y)(OR¹⁰)(OR¹¹), —OP(OR¹⁰)(OR¹¹), SR¹⁰, —S(O)R¹⁰, —S(O)₂R¹⁰, —S(O)₂NR¹⁰R¹¹, —S(O)(OR¹⁰), —S(O)₂(OR¹⁰), —SC(═Y)R¹⁰, —SC(═Y)OR¹⁰, —SC(═Y)NR¹⁰R¹¹, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, and C₁-C₂₀ heteroaryl;
R³ is a C₂-C₅ heterocyclyl, a C₂-C₅ heteroaryl, a fused bicyclic C₄-C₂₀ heterocyclyl or a fused bicyclic C₃-C₂₀ heteroaryl, each of which are unsubstituted or are optionally substituted;
R¹⁰, R¹¹ and R¹² are independently H, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl,
or R¹⁰ and R¹¹ together with the nitrogen to which they are attached optionally form a saturated, partially unsaturated or fully unsaturated C₃-C₂₀ heterocyclic ring optionally containing one or more additional ring atoms selected from N, O or S, wherein said heterocyclic ring is optionally substituted with one or more groups independently selected from oxo, (CH₂)ₘOR¹⁰NR¹⁰R¹¹, CF₃, F, Cl, Br, I, SO₂R¹⁰, C(═O)R¹⁰, NR¹²C(═Y)R¹¹, NR¹²S(O)₂R¹¹, C(═Y)NR¹⁰R¹¹, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl and C₁-C₂₀ heteroaryl;
R¹⁴ and R¹⁵ are independently selected from H, C₁-C₁₂ alkyl, or —(CH₂)ₙ-aryl,
or R¹⁴ and R¹⁵ together with the atoms to which they are attached form a saturated or partially unsaturated C₃-C₁₂ carbocyclic ring,
R¹⁶ and R¹⁷ are independently H, C₁-C₁₂ alkyl, C₂-C₈ alkenyl, C₂-C₈ alkynyl, C₃-C₁₂ carbocyclyl, or C₆-C₂₀ aryl,
R¹⁸ and R¹⁹ together with the carbon to which they are attached form a C₃-C₂₀ heterocyclic ring,
where said alkyl, alkenyl, alkynyl, carbocyclyl, heterocyclyl, aryl and heteroaryl are optionally substituted with one or more groups independently selected from F, Cl, Br, I, CN, CF₃, —NO₂, Oxo, R¹⁰, —C(═Y)R¹⁰, —C(═Y)OR¹⁰, —C(═Y)NR¹⁰R¹¹, —(CR¹⁴R¹⁵)ₙNR¹⁰R¹¹, —(CR¹⁴R¹⁵)OR¹⁰, —NR¹⁰R¹¹, —NR¹²C(═Y)R¹⁰, —NR¹²C(═Y)OR¹¹, —NR¹²C(═Y)NR¹⁰R¹¹, —NR¹²SO₂R¹⁰, ═NR¹², OR¹⁰, —OC(═Y)R¹⁰, —OC(═Y)OR¹⁰, —OC(═Y)NR¹⁰R¹¹, —OS(O)₂(OR¹⁰), —OP(═Y)(OR¹⁰)(OR¹¹), —OP(OR¹⁰)(OR¹¹), SR¹⁰, —S(O)R¹⁰, —S(O)₂R¹⁰, —S(O)₂NR¹⁰R¹¹, —S(O)(OR¹⁰), —S(O)₂(OR¹⁰), —SC(═Y)R¹⁰, —SC(═Y)OR¹⁰, —SC(═Y)NR¹⁰R¹¹, C₁-C₁₂ optionally substituted alkyl, C₂-C₈ optionally substituted alkenyl, C₂-C₈ optionally substituted alkynyl, C₃-C₁₂ optionally substituted carbocyclyl, C₂-C₂₀ optionally substituted heterocyclyl, C₆-C₂₀ optionally substituted aryl, C₁-C₂₀ optionally substituted heteroaryl, —(CR¹⁴R¹⁵)ₜ—NR¹²C(═O)(CR¹⁴R¹⁵)NR¹⁰R¹¹, and (CR¹⁴R¹⁵)ₜNR¹⁰R¹¹;

### Y is O, S, or NR¹²

m is 0, 1, 2, 3, 4, 5 or 6;
n is 1, 2, 3, 4, 5 or 6; and
t is 1, 2, 3, 4, 5 or 6.

In the above formula (VIII), the substituent groups that are alkyls, alkenyls, alkynyls, carbocyclyls, heterocyclyls, aryls, heteroaryls, fused bicyclic heterocyclyls, and fused bicyclic heteroaryls are optionally substituted with one or more substituents selected from the group consisting of F, Cl, Br, I, CN, CF₃, —NO₂, —NH₂, oxo, R¹⁰, —C(═Y)R¹⁰, —C(═Y)OR¹⁰, —C(═Y)NR¹⁰R¹¹, —(CR¹⁴R¹⁵)ₙNR¹⁰R¹¹, —(CR¹⁴R¹⁵)ₙOR¹⁰, —NR¹⁰R¹¹, —NR¹²C(═Y)R¹⁰, —NR¹²C(═Y)OR¹¹, —NR¹²C(═Y)NR¹⁰R¹¹, —NR¹²SO₂R¹⁰, ═NR¹², OR¹⁰, —OC(═Y)R¹⁰, —OC(═Y)OR¹⁰, —OC(═Y)NR¹⁰R¹¹, —OS(O)₂(OR¹⁰), —OP(═Y)(OR¹⁰)(OR¹¹), —OP(OR¹⁰)(OR¹¹), SR¹⁰, —S(O)R¹⁰, —S(O)₂R¹⁰, —S(O)₂NR¹⁰R¹¹, —S(O)(OR¹⁰), —S(O)₂(OR¹⁰), —SC(═Y)R¹⁰, —SC(═Y)OR¹⁰, —SC(═Y)NR¹⁰R¹¹, C₁-C₁₂ optionally substituted alkyl, C₂-C₈ optionally substituted alkenyl, C₂-C₈ optionally substituted alkynyl, C₃-C₁₂ optionally substituted carbocyclyl, C₂-C₂₀ optionally substituted heterocyclyl, C6-C20 optionally substituted aryl, C1-C20 optionally substituted heteroaryl, —(CR¹⁴R¹⁵)ₜ—NR¹²C(═O)(CR¹⁴R¹⁵)NR¹⁰R¹¹, and (CR¹⁴R¹⁵)ₜ—NR¹⁰R¹¹

Compounds of Formula VIII include GNE-493 and GNE-0941 (structures disclosed above), GNE-490 and GNE-477.

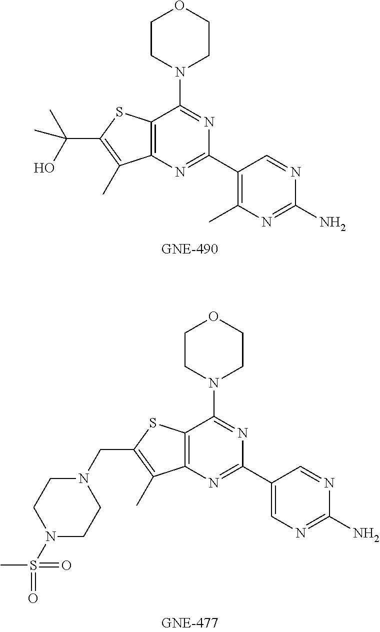 [SMILES: Cc1nc(N)ncc1-c1nc(N2CCOCC2)c2sc(C(C)(C)O)c(C)c2n1 ; Cc1c(CN2CCN(S(C)(=O)=O)CC2)sc2c(N3CCOCC3)nc(-c3cnc(N)nc3)nc12]

The invention further provides mTOR inhibitors of Formula IX:

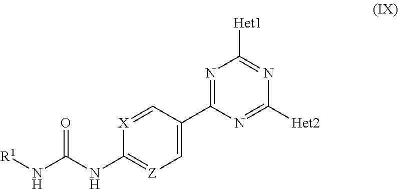

wherein,
Het1 and Het2 are independently selected from (i) a 5 to 7 membered heterocyclic group having 1-3 heteroatoms selected from O, N, and S, and (ii) a 6 to 10 membered bicyclic heterocyclic group having 1-3 heteroatoms selected from O, N, and S; wherein each of (i) or (ii) may by unsubstituted or substituted with 1 to 3 substituents selected from C₁-C₄ alkyl, NH₂, —O—R⁰, CN, and halo;
X and Z are independently selected from N and CH;
R¹ is selected from the group consisting of
(i) C₁-C₁₀ alkyl, C₂-C₁₀ alkenyl, C₂-C₁₀ alkynyl, and C₃ to C₁₀ cycloalkyl; each of which is unsubstituted or is substituted by 1 to 3 substituents selected from the group consisting of halo, —N(R⁰)₂, CN, NO₂, —O—(C₂-C₁₀ alkyl), aryl, heteroaryl, and heterocyclyl;
(ii) aryl, heteroaryl and heterocyclyl; each of which is unsubstituted or is substituted by 1 to 3 substituents selected from the group consisting of halo, —N(R⁰)₂, CN, NO₂, C₁-C₆ alkyl, C₂-C₆ alkenyl, aryl, heteroaryl, heterocyclyl, —O—R⁰, —(C₁-C₁₀ alkyl)-OR⁰, —O—(C₁-C₁₀ alkyl)-OR⁰, —(C₁-C₁₀ alkyl)-N(R⁰)₂, —O—(C₂-C₁₀ alkyl)-N(R⁰)₂, —O—(C₂-C₁₀ alkyl)-C(═O)—N(R⁰)₂, —C(═O)(C₂-C₁₀ alkyl)-N(R⁰)₂, —C(═O)N(R⁰)—(C₂-C₁₀ alkyl)-N(R⁰)₂, and —(C₁-C₆ alkyl)-aryl;
(iii) -aryl-C(═O)-aryl, -aryl-C(═O)-heteroaryl, and -aryl-C(═O)-heterocyclyl; each of which is unsubstituted or is substituted by 1 to 3 substituents selected from the group consisting of halo, —N(R⁰)₂, CN, NO₂, C₁-C₆ alkyl, C₂-C₆ alkenyl, and —O—R⁰;
(iv) -aryl-aryl, -aryl-heteroaryl, and -aryl-heterocyclyl; each of which is unsubstituted or is substituted by 1 to 3 substituents selected from the group consisting of halo, —N(R⁰)₂, CN, NO₂, C₁-C₆ alkyl, C₂-C₆ alkenyl, —O—R⁰; and
   each R⁰ is independently selected from H and C₁-C₆ alkyl; or alternatively two adjacent R⁰ attached to a nitrogen atom may be taken together to form a 5 or 6-membered heterocyclic ring having 0 to 2 additional ring heteroatoms selected from N, O, and S, and which may be unsubstituted or substituted by 1 to 3 substituents selected form the group consisting of C₁-C₄ alkyl, NH₂, —OH, CN, and halo.

Compounds of Formula IX also include compounds 9-13 disclosed by Zask et al, Bioorg. Med. Chem. Let. (2010) 20:2644-2647 and compounds I-15, and 18-39 disclosed by Verheijen et al, Bioorg. Med. Chem. Let. (2010) 20:2648-2653. These compounds of Formula IX include Wyeth BMCL 20102644-13 and Wyeth BMCL 20102648-27.

The invention further provides mTOR and mTOR/PI3K inhibitors of Formula X:

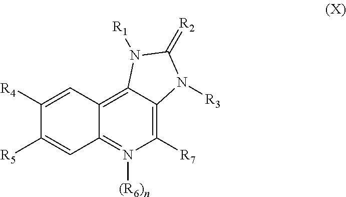

wherein
R₁ is selected from naphthyl or phenyl, which is unsubstituted or is substituted by one or two substituents independently selected from the group consisting of (i) halogen; (ii) lower alkyl unsubstituted or substituted by halogen, cyano, imidazolyl or triazolyl; (iii) cycloalkyl; (iv) amino substituted by one or two substituents independently selected from the group consisting of lower alkyl, lower alkyl sulfonyl, lower alkoxy and lower alkoxy lower alkylamino; (v) piperazinyl unsubstituted or substituted by one or two substituents independently selected from the group consisting of lower alkyl and lower alkyl sulfonyl; (vi) 2-oxo-pyrrolidinyl; (vii) lower alkoxy lower alkyl; (viii) imidazolyl; (ix) pyrazolyl; and (x) triazolyl;

### R₂ is O or S;

R₃ is lower alkyl;
R₄ is selected from (i) pyridyl unsubstituted or substituted by halogen, cyano, lower alkyl, lower alkoxy or piperazinyl unsubstituted or substituted by lower alkyl; (ii) pyrimidinyl unsubstituted or substituted by lower alkoxy; (iii) quinolinyl unsubstituted or substituted by halogen; or (iv) quinoxalinyl;
R₅ is hydrogen or halogen;
n is 0 or 1;
R₆ is oxido; with the proviso that if n=1, the N-atom bearing the radical R₆ has a positive charge; and
R₇ is hydrogen or amino.

NVP-BEZ235 is a compound of Formula X and has the following chemical structure:

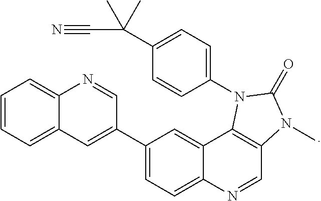 [SMILES: Cn1c(=O)n(-c2ccc(C(C)(C)C#N)cc2)c2c3cc(-c4cnc5ccccc5c4)ccc3ncc21]

In addition to the compounds disclosed above, selective mTOR inhibitors that can be used in the present invention include KU-BMCL-200908069-1; KU-BMCL-200908069-5 (IC₅₀ 21 nmol; >500-fold selective versus PI3Ks); WAY-600 (IC₅₀ 9 nmol; >100-fold selective versus PI3Kα and >500 selective versus PI3Kγ); WYE-687 (IC₅₀ 7 nmol; >100-fold selective versus PI3Kα and >500 selective versus PI3Kγ); WYE-354 (IC₅₀ 5 nmol; >100-fold selective versus PI3Kα and >500 selective versus PI3Kγ); Wyeth-BMCL-200910075-9b (IC₅₀ 0.7 nmol; >1,000-fold selective versus PI3K); Wyeth-BMCL-200910096-27 (IC₅₀ 0.6 nmol; >200-fold selective versus PI3Kα); INK128 (Intellikine, Inc.) (IC₅₀ 1 nmol; >100-fold selective versus PI3Ks); XL388 (Exelixis) (IC₅₀ 9.8 nmol against mTORC1 and 166 nM against mTORC2; >100-fold selective versus a panel of 140 protein kinases (IC₅₀>3 μM)); AZD8055 (Astra Zeneca) (IC₅₀ 0.13 nmol; >10,000-fold selective versus p100α); and OSI-027 (OSI pharmaceuticals). Another ATP-competitive specific mTOR inhibitor is WYE-125132 (IC₅₀ 0.19 nmol; >5,000-fold selective versus PI3Ks). Other mTOR inhibitors that can be used in the present invention include those disclosed in WO2006/090167, WO2006/090169, WO2007/060404, WO2007/080382, and WO2008/023161.

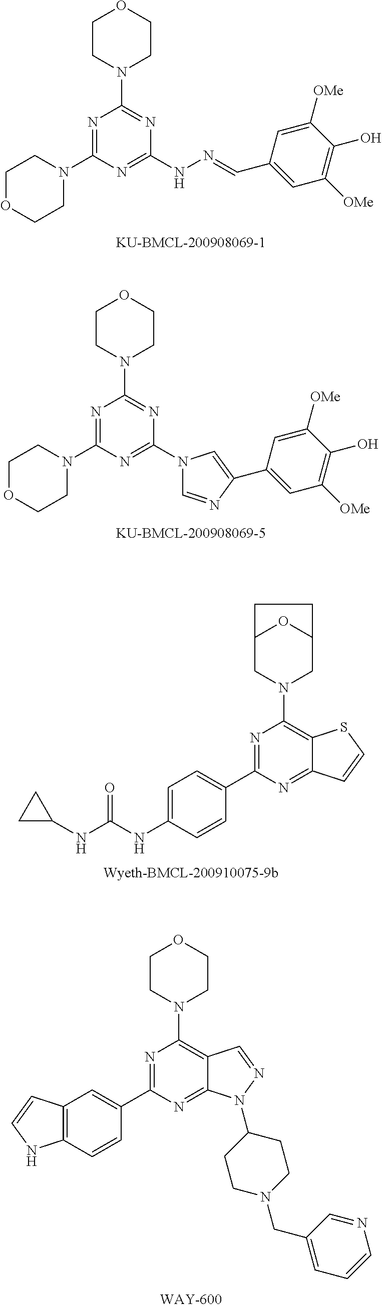 [SMILES: COc1cc(/C=N/Nc2nc(N3CCOCC3)nc(N3CCOCC3)n2)cc(OC)c1O ; COc1cc(-c2cn(-c3nc(N4CCOCC4)nc(N4CCOCC4)n3)cn2)cc(OC)c1O ; O=C(Nc1ccc(-c2nc(N3CC4CCC(C3)O4)c3sccc3n2)cc1)NC1CC1 ; c1cncc(CN2CCC(n3ncc4c(N5CCOCC5)nc(-c5ccc6[nH]ccc6c5)nc43)CC2)c1]

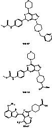 [SMILES: COC(=O)Nc1ccc(-c2nc(N3CCOCC3)c3cnn(C4CCN(Cc5cccnc5)CC4)c3n2)cc1 ; COC(=O)Nc1ccc(-c2nc(N3CCOCC3)c3cnn(C4CCN(C(=O)OC)CC4)c3n2)cc1 ; COc1cccc2cc(-c3nc([C@H]4CC[C@H](C(=O)O)CC4)n4ncnc(N)c34)[nH]c12]

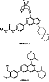 [SMILES: CNC(=O)Nc1ccc(-c2nc(N3C[C@H]4CC[C@@H](C3)O4)c3cnn(C4CCC5(CC4)OCCO5)c3n2)cc1 ; COc1ccc(-c2ccc3c(N4CCOC[C@@H]4C)nc(N4CCOC[C@@H]4C)nc3n2)cc1CO]

Additional mTOR inhibitors and dual mTOR/PI3K inhibitors that can be used in the present invention include: AZD2014 (Zask et al., Expert Opin Ther. Patents (2011) 21:11109-1127); CC-223 (TORKi) (Zask et al. (2011)): SB-2015 (Bonday, American Association for Cancer Research Annual Meeting, Denver, Colo., USA (2009) 100:Abs 3710); NVP-BGT-226 (Zask et al. (2011); Richards et al., Curr. Opin. in Drug Disc. & Dev. (2010) 13:428-440); and the following compounds:

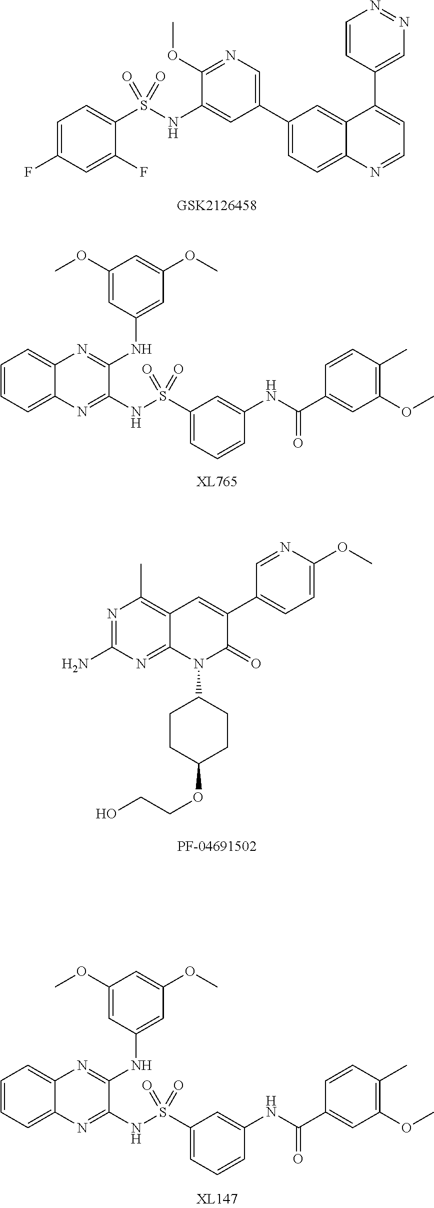

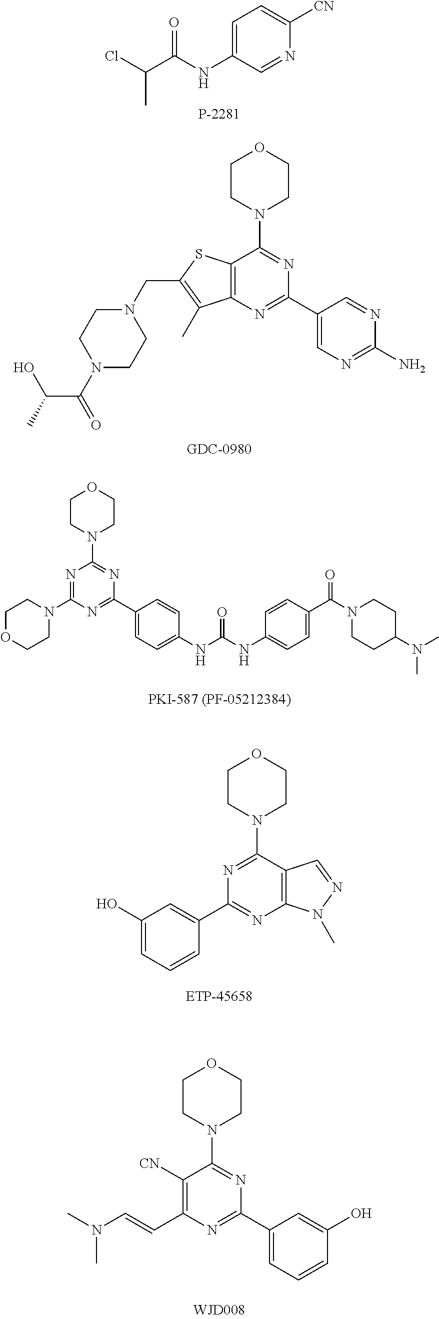

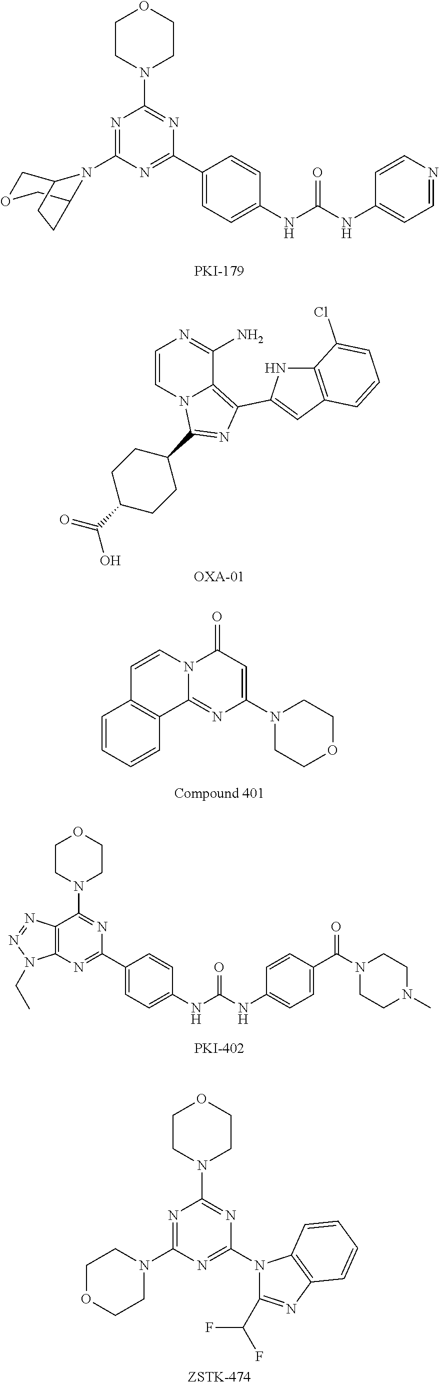

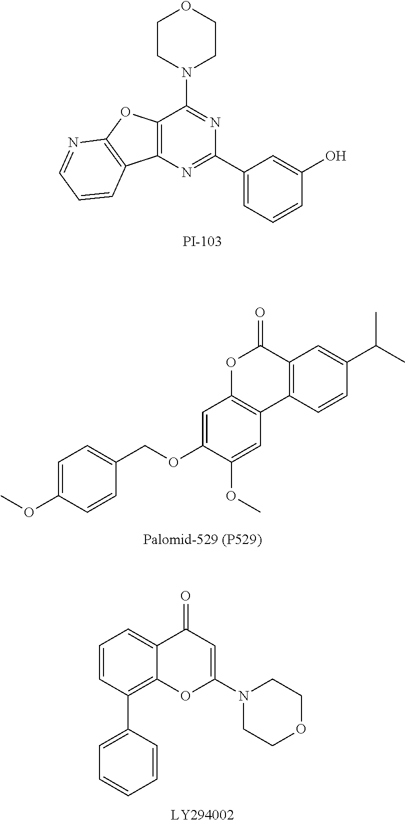 [SMILES: Oc1cccc(-c2nc(N3CCOCC3)c3oc4ncccc4c3n2)c1 ; COc1ccc(COc2cc3oc(=O)c4cc(C(C)C)ccc4c3cc2OC)cc1 ; O=c1cc(N2CCOCC2)oc2c(-c3ccccc3)cccc12]

The following mTOR inhibitors (WO 2010/051043) can also be used according to the invention.

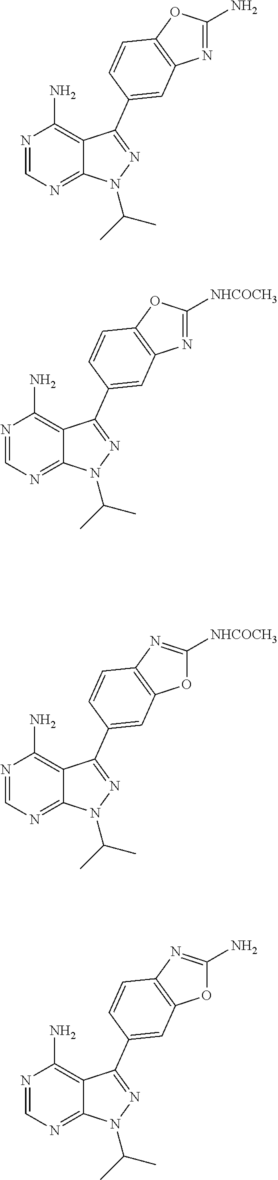

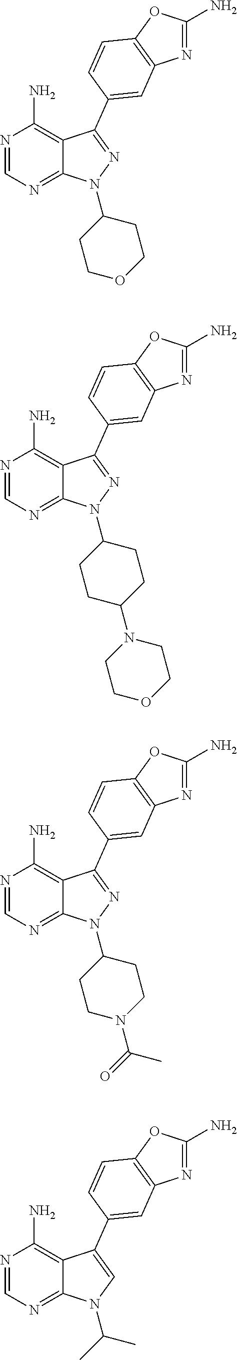 [SMILES: Nc1nc2cc(-c3nn(C4CCOCC4)c4ncnc(N)c34)ccc2o1 ; Nc1nc2cc(-c3nn(C4CCC(N5CCOCC5)CC4)c4ncnc(N)c34)ccc2o1 ; CC(=O)N1CCC(n2nc(-c3ccc4oc(N)nc4c3)c3c(N)ncnc32)CC1 ; CC(C)n1cc(-c2ccc3oc(N)nc3c2)c2c(N)ncnc21]

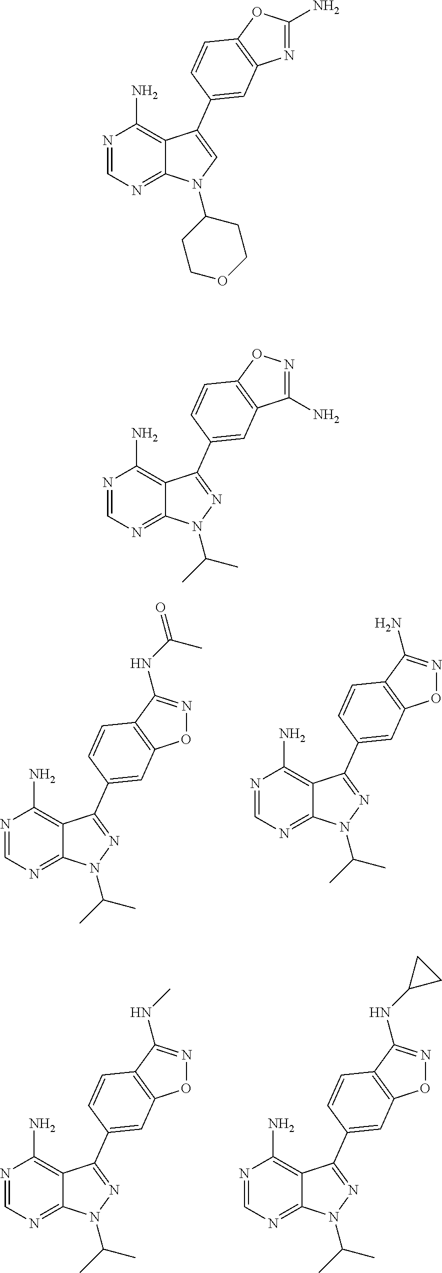

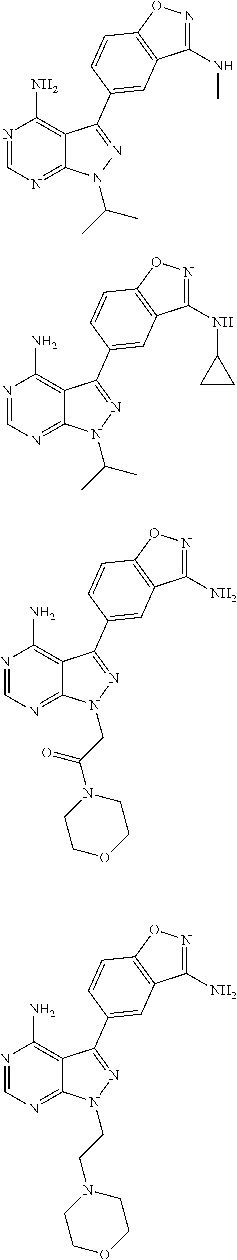 [SMILES: CNc1noc2ccc(-c3nn(C(C)C)c4ncnc(N)c34)cc12 ; CC(C)n1nc(-c2ccc3onc(NC4CC4)c3c2)c2c(N)ncnc21 ; Nc1noc2ccc(-c3nn(CC(=O)N4CCOCC4)c4ncnc(N)c34)cc12 ; Nc1noc2ccc(-c3nn(CCN4CCOCC4)c4ncnc(N)c34)cc12]

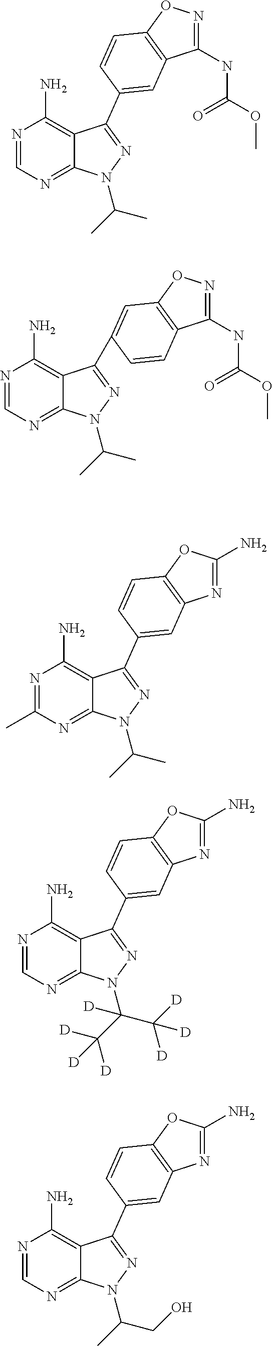

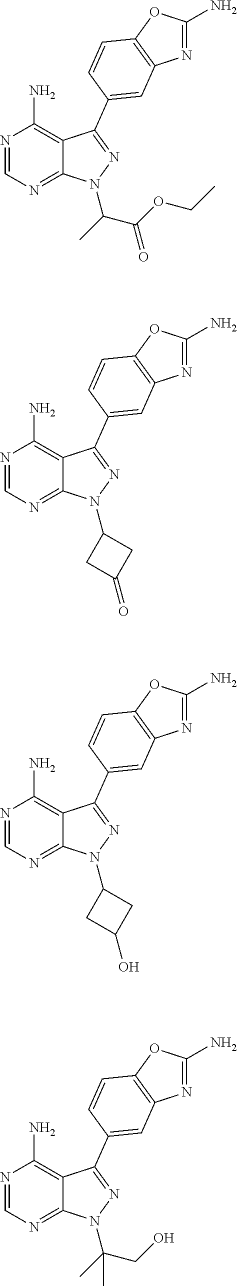 [SMILES: CCOC(=O)C(C)n1nc(-c2ccc3oc(N)nc3c2)c2c(N)ncnc21 ; Nc1nc2cc(-c3nn(C4CC(=O)C4)c4ncnc(N)c34)ccc2o1 ; Nc1nc2cc(-c3nn(C4CC(O)C4)c4ncnc(N)c34)ccc2o1 ; CC(C)(CO)n1nc(-c2ccc3oc(N)nc3c2)c2c(N)ncnc21]

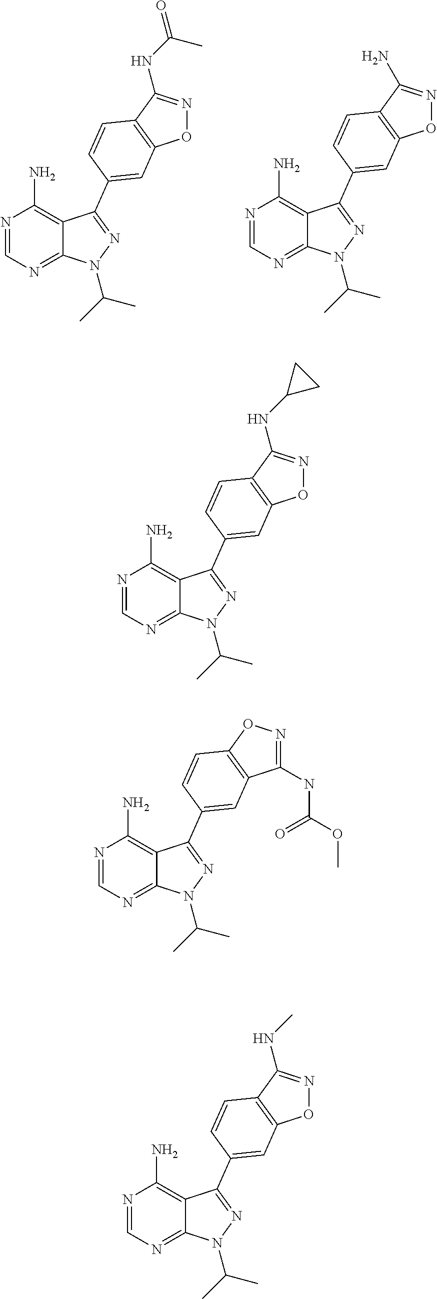 [SMILES: CC(=O)Nc1noc2cc(-c3nn(C(C)C)c4ncnc(N)c34)ccc12 ; CC(C)n1nc(-c2ccc3c(N)noc3c2)c2c(N)ncnc21 ; CC(C)n1nc(-c2ccc3c(NC4CC4)noc3c2)c2c(N)ncnc21 ; COC(=O)Nc1noc2ccc(-c3nn(C(C)C)c4ncnc(N)c34)cc12 ; CNc1noc2cc(-c3nn(C(C)C)c4ncnc(N)c34)ccc12]

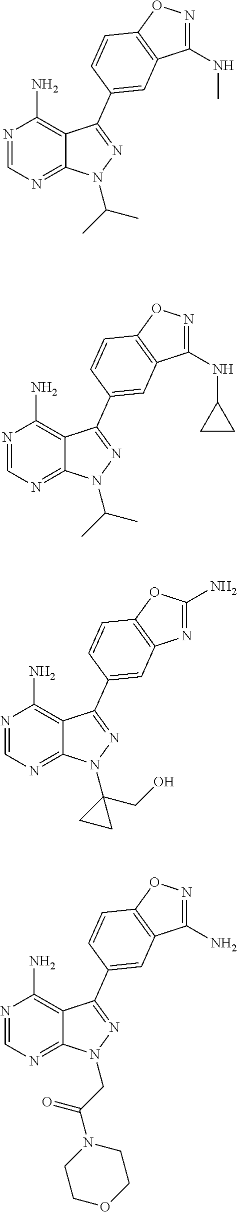 [SMILES: CNc1noc2ccc(-c3nn(C(C)C)c4ncnc(N)c34)cc12 ; CC(C)n1nc(-c2ccc3onc(NC4CC4)c3c2)c2c(N)ncnc21 ; Nc1nc2cc(-c3nn(C4(CO)CC4)c4ncnc(N)c34)ccc2o1 ; Nc1noc2ccc(-c3nn(CC(=O)N4CCOCC4)c4ncnc(N)c34)cc12]

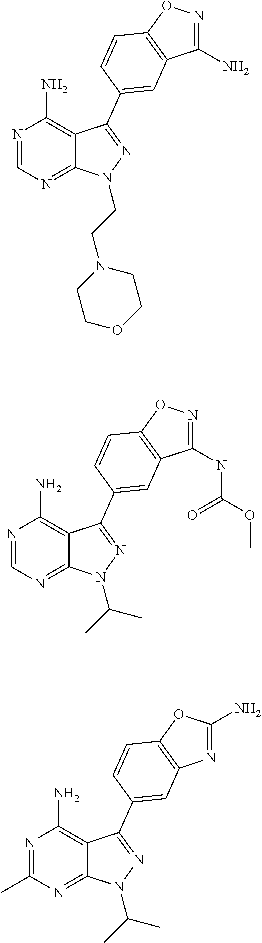

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art, See for example, Remington's Pharmaceutical Sciences, 18th eds., Mack Publishing, Easton Pa. (1990) or Remington: The Science and Practice of Pharmacy, 19th eds., Mack Publishing, Easton Pa. (1995).

As used herein and unless otherwise indicated, the term “hydrate” means a compound, or a salt thereof, that further includes a stoichiometric or non-stoichiometric amount of water bound by non-covalent intermolecular forces.

As used herein and unless otherwise indicated, the term “solvate” means a compound, or a salt thereof, that further includes a stoichiometric or non-stoichiometric amount of a solvent bound by non-covalent intermolecular forces.

As used herein and unless otherwise indicated, the term “prodrug” means a compound derivative that can hydrolyze, oxidize, or otherwise react under biological conditions (in vitro or in vivo) to provide a compound. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound, in the context of an organic or inorganic molecule, that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof.

Various compounds contain one or more chiral centers, and can exist as racemic mixtures of enantiomers, mixtures of diastereomers or enantiomerically or optically pure compounds. The use of stereomerically pure forms of such compounds, as well as the use of mixtures of those forms are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

It should also be noted that compounds, in the context of organic and inorganic molecules, can include E and Z isomers, or a mixture thereof, and cis and trans isomers or a mixture thereof. In certain embodiments, compounds are isolated as either the E or Z isomer. In other embodiments, compounds are a mixture of the E and Z isomers.

According to the invention, an inhibitor of a rapamycin-resistant function of mTOR or a related compound or analog or prodrug thereof, is used for treating or preventing infection by a virus that depends on maintaining mTOR function for replication and/or spread. In one embodiment, an inhibitor of a rapamycin-resistant function of mTOR or a related compound or analog or prodrug thereof, is used for treating or preventing infection by a herpesvirus. Herpesvirus (Herpesviridae) is a family of viruses that contain a double stranded DNA genome. For example, as exemplified herein, nanomolar concentrations of torin1 inhibit the replication of herpes simplex virus-1 (HSV-1), which is an α-herpesvirus; human cytomegalovirus (HCMV), which is a β-herpesvirus; and γ-herpesvirus 68, which is a γ-herpesvirus.

As used herein, the term “effective amount” in the context of administering a therapy to a subject refers to the amount of a therapy which is sufficient to achieve one, two, three, four, or more of the following effects: (i) reduce or ameliorate the severity of a viral infection or a symptom associated therewith; (ii) reduce the duration of a viral infection or a symptom associated therewith; (iii) prevent the progression of a viral infection or a symptom associated therewith; (iv) cause regression of a viral infection or a symptom associated therewith; (v) prevent the development or onset of a viral infection or a symptom associated therewith; (vi) prevent the recurrence of a viral infection or a symptom associated therewith; (vii) reduce or prevent the spread of a virus from one cell to another cell, or one tissue to another tissue; (ix) prevent or reduce the spread of a virus from one subject to another subject; (x) reduce organ failure associated with a viral infection; (xi) reduce hospitalization of a subject; (xii) reduce hospitalization length; (xiii) increase the survival of a subject with a viral infection; (xiv) eliminate a virus infection; and/or (xv) enhance or improve the prophylactic or therapeutic effect(s) of another therapy.

As used herein, the term “effective amount” in the context of a compound for use in cell culture-related products refers to an amount of a compound which is sufficient to reduce the viral titer in cell culture or prevent the replication of a virus in cell culture.

A preferred dose of an mTOR inhibitor used to treat or prevent viral infections in mammals is <100 mg/kg, <50 mg/kg, <20 mg/kg, <10 mg/kg, <5 mg/kg, <2 mg/kg, <1 mg/kg, <0.5 mg/kg, <0.2 mg/kg, <0.1 mg/kg, <0.05 mg/kg, <0.02 mg/kg, or <0.01 mg/kg. A preferred dose of an mTOR inhibitor used to treat or prevent viral infections in a mammal results in total serum concentrations of <100 μM, <50 μM, <20 μM, <10 μM, <5 μM, <1 μM, <500 nM, or <250 nM.

The present invention also provides for the use of an mTOR inhibitor in cell culture-related products in which it is desirable to have antiviral activity. In one embodiment, an mTOR inhibitor is added to cell culture media. An mTOR inhibitor used in cell culture media includes compounds that may otherwise be found too toxic for treatment of a subject.

### 2. Inhibitors of the Unfolded Protein Response

In one embodiment, the present invention provides a method of treating or preventing a viral infection in a mammalian subject, comprising administering to a subject in need thereof a therapeutically effective amount of a compound that inhibits the Unfolded Protein Response (UPR). In one embodiment, the inhibitors of UPR combined with mTOR inhibitors to treat or prevent viral infection.

Viral protein synthesis, including the synthesis of virus-coded glycoproteins, increases dramatically as infection progresses. When the synthesis of glycoproteins exceeds the capacity of the cell to properly fold and traffic these proteins, the cell induces a stress response referred to as the unfolded protein response, or UPR. The mechanisms by which the UPR resolves cell stress are multi-faceted. They include the increased expression of chaperone proteins, the increased expression of proteins that resolve cell stress, and a reduction in the global rate of protein synthesis. In combination, these UPR events act to maintain cellular homeostasis. In the presence of stress and the absence of the UPR, cells induce a set of events resulting in cell death.

Thus, in one embodiment, compounds of the invention act as chemical chaperones and inhibit the UPR. One such chemical chaperone is 4-phenylbutyrate (4-PBA). Other chemical chaperones include taurourodeoxycholic acid (TUDCA), trimethylamine trioxide (TMO) and betaine.

A preferred dose of an inhibitor of the UPR used to treat or prevent viral infections in mammals is <100 mg/kg, <50 mg/kg, <20 mg/kg, <10 mg/kg, <5 mg/kg, <2 mg/kg, <1 mg/kg, <0.5 mg/kg, <0.2 mg/kg, <0.1 mg/kg, <0.05 mg/kg, <0.02 mg/kg, or <0.01 mg/kg. A preferred dose of an UPR inhibitor used to treat or prevent a viral infection in a mammal results in total serum concentrations of <100 μM, <50 μM, <20 μM, <10 μM, <5 μM, <1 μM, <500 nM, or <250 nM.

The present invention also provides for the use of an inhibitor of the UPR in cell culture-related products in which it is desirable to have antiviral activity. In one embodiment, an inhibitor of the UPR is added to cell culture media. An inhibitor of the UPR used in cell culture media includes compounds that may otherwise be found too toxic for treatment of a subject.

3. Combination of Inhibitors of mTOR and Inhibitors of the Unfolded Protein Response

In one embodiment, the present invention provides a method of treating or preventing a viral infection in a mammal, comprising administering to a subject in need thereof a therapeutically effective amount of a combination of a first compound or a relative, analogue, or derivative thereof, wherein the first compound is an inhibitor of mTOR and a second compound or a relative, analogue, or derivative thereof, wherein the second compound is an inhibitor of the UPR. In one embodiment, the mTOR inhibitor used in combination with the inhibitor of the UPR is a specific inhibitor of mTOR. In other embodiments the mTOR inhibitor is less specific with significant activity against other protein kinases such as XL765, PI-103, PF-4691502, LY294002, and LOR-220. In other embodiments, the inhibitor of mTOR inhibits a rapamycin-resistant function of mTOR, a rapamycin-sensitive sensitive function of mTOR, or both.

Thus, in addition to the mTOR inhibitors described in section 1, mTOR inhibitors that can be used in combination with inhibitors of the UPR include rapamycin and its analogs (rapalogs) such as: norrapamycin, everolimus, temsirolimus (CCI-779), ridaforolimus (AP23573), zotarolimus, deoxorapamycin, desmethylrapamycins, desmethoxyrapamycins, AP22594, 28-epi-rapamycin, 24,30-tetrahydro-rapamycin, ridaforolimus (AP23573), trans-3-aza-bicyclo[3.1.0]hexane-2-carboxylic acid rapamycin, ABT-578, SDZ RAD, AP20840, AP23464, AP23675, AP23841, AP24170, TAFA93, 40-O-(2-hydroxyethyl)-rapamycin, 32-deoxorapamycin, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, 40-[3-hydroxy-2-(hydroxy-methyl)-2-methylpropanoate]-rapamycin (CC1779), 40-epi-(tetrazolyl)-rapamycin (ABT578), TAFA-93, biolimus-7, biolimus-9, biolimus A9 and combinations.

As used herein, the term “combination,” in the context of the administration of two or more therapies to a subject, refers to the use of more than one therapy (e.g., more than one prophylactic agent and/or therapeutic agent). The use of the term “combination” does not restrict the order in which therapies are administered to a subject with a viral infection. A first therapy (e.g., a first prophylactic or therapeutic agent) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject with a viral infection.

4. Screening Assays to Identify Inhibitors of mTOR

Compounds known to be inhibitors of a rapamycin-resistant function of mTOR can be directly screened for antiviral activity using assays known in the art and/or described herein. While optional, derivatives or congeners of such inhibitors, or any other compound can be tested for their ability to modulate mTOR function using assays known to those of ordinary skill in the art and/or described below. Compounds found to modulate mTOR function can be further tested for antiviral activity.

Alternatively, compounds can be tested directly for antiviral activity. Those compounds which demonstrate anti-viral activity, or that are known to be antiviral but have unacceptable specificity or toxicity, can be screened for mTOR inhibitory activity. Antiviral compounds that modulate the enzyme targets can be optimized for better activity profiles.

Assays to test compounds for mTOR kinase activity are known in the art (see e.g., Yu et al. Cancer Res. (2009) 69:6232-6240; Thoreen et al., J. Biological Chemistry (2009) 284:8023-8032; Reichling et al. J. Biomol Screen. (2008) 13:238-244).

To determine the selectivity of a compound for inhibition of mTOR kinase activity, the compound can be tested for inhibition of the kinase activity of a panel of kinases including, for example, one or more kinases listed in Table 1.

TABLE 1
Examples of kinases that may be tested to
determine selectivity of the mTOR inhibitor.
PIK3C2B
PIK3CA
PIK3CA (E545K)
PIK3CB
PIK3CD
PIK3CG
PI4Kβ
DNA-PK
PDK1
PKCα
PKCβI
PKCβII
RET
RAF1
JAK1
JAK2
JNK1
JNK2
JNK3

Methods for testing inhibition of protein kinases, such as serine/threonine kinases, and lipid kinases, such as PI3K, are known in the art (see e.g., Zask et al. J. Med. Chem. (2008) 51:1319-1323; Yu et al. Cancer Res. (2009) 69:6232-6240; Thoreen et al., J. Biological Chemistry (2009) 284:8023-8032).

For example, lipid kinase assays are described in Thoreen et al., J. Biological Chemistry (2009) 284:8023-8032. Reactions are performed in triplicate with variable amounts of inhibitor and with 10 μM ATP, 2 mM DTT, and a kinase-specific buffer and substrate. 50 μM PIP2:PS lipid kinase substrate can be used for p110α/p85α, p110β/p85α and p110γ. 100 μM PIP2:PS lipid kinase substrate can be used for p110δ/p85α. 100 μM PI lipid kinase substrate can be used for PI3K-C2α and PI3K-C2β. 100 μM PI:PS lipid kinase substrate can be used for hVPS34. The buffer for p110δ/p58α, p110β/p85α, p110δ/p85α, PI3K-C2α, and PI3K-C2β is 50 mM Hepes pH 7.5, 3 mM MgCl₂, 1 mM EGTA, 100 mM NaCl, and 0.03% CHAPS. The buffer for hVPS34 was 50 mM Hepes pH 7.3, 0.1% CHAPS, 1 mM EGTA, and 5 mM MnCl₂. The enzyme concentrations are 0.12, 4.5, 0.79, 3.5, 6.3, 42, and 2.8 nM for p110α/p85α, p110β/p85α, p110δ/p58α, p110γ, PI3K-C2α, PI3K-C2β, and hVPS34, respectively. After 1 hour at room temperature, 5 μL of detection mix is added, comprised of 12 nM Alexa Fluor647® ADP Tracer, 6 nM Adapta™ Eu-anti-ADP Antibody, 20 mM Tris pH 7.5, 0.01% NP-40, and 30 mM EDTA. After 30 minutes, the plates can be read on a Tecan InfiniTE® F500 or BMG PHERAstar plate reader. Instrument settings suitable for Adatpa™ assays (Invitrogen) are used measuring emission at 665 and 615 nm after excitation at 340 nm and with a lag time of 100 s and integration time of 200 s. The raw emission ratio (emission at 665 nm÷emission at 615 nm) values are converted to product formation (% conversion of ATP) using nucleotide (ATP:ADP) standard curves. IC₅₀ values are calculated from plots of compound concentration versus product formation.

Any host cell enzyme, that relates to a rapamycin resistant function of mTOR, is contemplated as a potential target for antiviral intervention. Further, additional host cell enzymes that have a role, directly or indirectly, in regulating the cell's translation activity are contemplated as potential targets for antiviral intervention.

In some embodiments of the invention, the compound increases an enzyme's activity (for example, an enzyme that is a negative regulator of mTOR might have its activity increased by a potential antiviral compound). In specific embodiments, the compound increases an enzyme's activity by at least approximately 10%, 15%, 20%, 25%, 30%, 40%, 50%, 60%, 70%, 80% or 90%. In some embodiments, the compound decreases an enzyme's activity. In particular embodiments, the compound decreases an enzyme's activity by at least approximately 10%, 15%, 20%, 25%, 30%, 40%, 50%, 60%, 70%, 80%, 90%, 95% or 100%. In certain embodiments, the compound exclusively modulates a single enzyme. In some embodiments, the compound modulates multiple enzymes, although it might modulate one enzyme to a greater extent than another. Using the standard enzyme activity assays described herein, the activity of the compounds could be characterized. In one embodiment, a compound exhibits an irreversible inhibition or activation of a particular enzyme. In some embodiments, a compound reversibly inhibits or activates an enzyme. In some embodiments, a compound alters the kinetics of the enzyme.

In one embodiment, for example, evaluating the interaction between the test compound and host target enzyme includes one or more of (i) evaluating binding of the test compound to the enzyme; (ii) evaluating a biological activity of the enzyme; (iii) evaluating an enzymatic activity (e.g., kinase activity) of the enzyme in the presence and absence of test compound. The in vitro contacting can include forming a reaction mixture that includes the test compound, enzyme, any required cofactor (e.g., biotin) or energy source (e.g., ATP, or radiolabeled ATP), a substrate (e.g., acetyl-CoA, a sugar, a polypeptide, a nucleoside, or any other metabolite, with or without label) and evaluating conversion of the substrate into a product. Evaluating product formation can include, for example, detecting the transfer of carbons or phosphate (e.g., chemically or using a label, e.g., a radiolabel), detecting the reaction product, detecting a secondary reaction dependent on the first reaction, or detecting a physical property of the substrate, e.g., a change in molecular weight, charge, or pI.

Target enzymes for use in screening assays can be purified from a natural source, e.g., cells, tissues or organs comprising adipocytes (e.g., adipose tissue), liver, etc. Alternatively, target enzymes can be expressed in any of a number of different recombinant DNA expression systems and can be obtained in large amounts and tested for biological activity. For expression in recombinant bacterial cells, for example E. coli, cells are grown in any of a number of suitable media, for example LB, and the expression of the recombinant polypeptide induced by adding IPTG to the media or switching incubation to a higher temperature. After culturing the bacteria for a further period of between 2 and 24 hours, the cells are collected by centrifugation and washed to remove residual media. The bacterial cells are then lysed, for example, by disruption in a cell homogenizer and centrifuged to separate the dense inclusion bodies and cell membranes from the soluble cell components. This centrifugation can be performed under conditions whereby the dense inclusion bodies are selectively enriched by incorporation of sugars such as sucrose into the buffer and centrifugation at a selective speed. If the recombinant polypeptide is expressed in the inclusion, these can be washed in any of several solutions to remove some of the contaminating host proteins, then solubilized in solutions containing high concentrations of urea (e.g., 8 M) or chaotropic agents such as guanidine hydrochloride in the presence of reducing agents such as beta-mercaptoethanol or DTT (dithiothreitol). At this stage it may be advantageous to incubate the polypeptide for several hours under conditions suitable for the polypeptide to undergo a refolding process into a conformation which more closely resembles that of the native polypeptide. Such conditions generally include low polypeptide (concentrations less than 500 mg/ml), low levels of reducing agent, concentrations of urea less than 2 M and often the presence of reagents such as a mixture of reduced and oxidized glutathione which facilitate the interchange of disulphide bonds within the protein molecule. The refolding process can be monitored, for example, by SDS-PAGE or with antibodies which are specific for the native molecule. Following refolding, the polypeptide can then be purified further and separated from the refolding mixture by chromatography on any of several supports including ion exchange resins, gel permeation resins or on a variety of affinity columns.

Isolation and purification of host cell expressed polypeptide, or fragments thereof may be carried out by conventional means including, but not limited to, preparative chromatography and immunological separations involving monoclonal or polyclonal antibodies.

These polypeptides may be produced in a variety of ways, including via recombinant DNA techniques, to enable large scale production of pure, biologically active target enzyme useful for screening compounds for the purposes of the invention. Alternatively, the target enzyme to be screened could be partially purified or tested in a cellular lysate or other solution or mixture.

Substrate and product levels can be evaluated in an in vitro system, e.g., in a biochemical extract, e.g., of proteins. For example, the extract may include all soluble proteins or a subset of proteins (e.g., a 70% or 50% ammonium sulfate cut), the useful subset of proteins defined as the subset that includes the target enzyme. The effect of a test compound can be evaluated, for example, by measuring substrate and product levels at the beginning of a time course, and then comparing such levels after a predetermined time (e.g., 0.5, 1, or 2 hours) in a reaction that includes the test compound and in a parallel control reaction that does not include the test compound. This is one method for determining the effect of a test compound on the substrate-to-product ratio in vitro. Reaction rates can obtained by linear regression analysis of radioactivity or other label incorporated vs. reaction time for each incubation. K_{M} and Vₘₐₓ values can be determined by non-linear regression analysis of initial velocities, according to the standard Henri-Michaelis-Menten equation. k_{cat} can be obtained by dividing Vₘₐₓ values by reaction concentrations of enzyme, e.g., derived by colorimetric protein determinations (e.g., Bio-RAD protein assay, Bradford assay, Lowry method). In one embodiment, the compound irreversibly inactivates the target enzyme. In another embodiment, the compound reversibly inhibits the target enzyme. In some embodiments, the compound reversibly inhibits the target enzyme by competitive inhibition. In some embodiments, the compound reversibly inhibits the target enzyme by noncompetitive inhibition. In some embodiments, the compound reversibly inhibits the target enzyme by uncompetitive inhibition. In a further embodiment, the compound inhibits the target enzyme by mixed inhibition. The mechanism of inhibition by the compound can be determined by standard assays known by those of ordinary skill in the art.

Methods for the quantitative measurement of enzyme activity utilizing a phase partition system are described in U.S. Pat. No. 6,994,956, which is incorporated by reference herein in its entirety. Specifically, a radiolabeled substrate and the product of the reaction are differentially partitioned into an aqueous phase and an immiscible scintillation fluid-containing organic phase, and enzyme activity is assessed either by incorporation of a radiolabeled-containing organic-soluble moiety into product molecules (gain of signal assay) or loss of a radiolabel-containing organic-soluble moiety from substrate molecules (loss of signal assay). Scintillations are only detected when the radionuclide is in the organic, scintillant-containing phase. Such methods can be employed to test the ability of a compound to inhibit the activity of a target enzyme.

Cellular assays may be employed. An exemplary cellular assay includes contacting a test compound to a culture cell (e.g., a mammalian culture cell, e.g., a human culture cell) and then evaluating substrate and product levels in the cell, e.g., using any method described herein, such as Reverse Phase HPLC, LC-MS, or LC-MS/MS.

Substrate and product levels can be evaluated, e.g., by NMR, HPLC (See, e.g., Bak, M. I., and Ingwall, J. S. (1994) J. Clin. Invest. 93, 40-49), mass spectrometry, thin layer chromatography, or the use of radiolabeled components (e.g., radiolabeled ATP for a kinase assay). For example, ³¹P NMR can be used to evaluate ATP and AMP levels. In one implementation, cells and/or tissue can be placed in a 10-mm NMR sample tube and inserted into a 1H/31P double-tuned probe situated in a 9.4-Tesla superconducting magnet with a bore of 89 cm. If desired, cells can be contacted with a substance that provides a distinctive peak in order to index the scans. Six ³¹P NMR spectra—each obtained by signal averaging of 104 free induction decays—can be collected using a 60° flip angle, 15-microsecond pulse, 2.14-second delay, 6,000 Hz sweep width, and 2048 data points using a GE-400 Omega NMR spectrometer (Bruker Instruments, Freemont, Calif., USA). Spectra are analyzed using 20-Hz exponential multiplication and zero- and first-order phase corrections. The resonance peak areas can be fitted by Lorentzian line shapes using NMR1 software (New Methods Research Inc., Syracuse, N.Y., USA). By comparing the peak areas of fully relaxed spectra (recycle time: 15 seconds) and partially saturated spectra (recycle time: 2.14 seconds), the correction factor for saturation can be calculated for the peaks. Peak areas can be normalized to cell and/or tissue weight or number and expressed in arbitrary area units. Another method for evaluating, e.g., ATP and AMP levels includes lysing cells in a sample to form an extract, and separating the extract by Reversed Phase HPLC, while monitoring absorbance at 260 nm.

Another type of in vitro assay evaluates the ability of a test compound to modulate interaction between a first enzyme pathway component and a second enzyme pathway component This type of assay can be accomplished, for example, by coupling one of the components with a radioisotope or enzymatic label such that binding of the labeled component to the second pathway component can be determined by detecting the labeled compound in a complex. An enzyme pathway component can be labeled with ¹²⁵I, ³⁵S, ¹⁴C, or ³H, either directly or indirectly, and the radioisotope detected by direct counting of radio-emission or by scintillation counting. Alternatively, a component can be enzymatically labeled with, for example, horseradish peroxidase, alkaline phosphatase, or luciferase, and the enzymatic label detected by determination of conversion of an appropriate substrate to product. Competition assays can also be used to evaluate a physical interaction between a test compound and a target.

Soluble and/or membrane-bound forms of isolated proteins (e.g., enzyme pathway components and their receptors or biologically active portions thereof) can be used in the cell-free assays of the invention. When membrane-bound forms of the enzyme are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton X-100, Triton X-114, Thesit, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl-N,N-dimethyl-3-ammonio-1-propane sulfonate. In another example, the enzyme pathway component can reside in a membrane, e.g., a liposome or other vesicle.

Cell-free assays involve preparing a reaction mixture of the target enzyme and the test compound under conditions and for a time sufficient to allow the two components to interact and bind, thus forming a complex that can be removed and/or detected. In one embodiment, the target enzyme is mixed with a solution containing one or more, and often many hundreds or thousands, of test compounds. The target enzyme, including any bound test compounds, is then isolated from unbound (i.e., free) test compounds, e.g., by size exclusion chromatography or affinity chromoatography. The test compound(s) bound to the target can then be separated from the target enzyme, e.g., by denaturing the enzyme in organic solvent, and the compounds identified by appropriate analytical approaches, e.g., LC-MS/MS.

The interaction between two molecules, e.g., target enzyme and test compound, can also be detected, e.g., using a fluorescence assay in which at least one molecule is fluorescently labeled, e.g., to evaluate an interaction between a test compound and a target enzyme. One example of such an assay includes fluorescence energy transfer (FET or FRET for fluorescence resonance energy transfer) (See, for example, Lakowicz et al., U.S. Pat. No. 5,631,169; Stavrianopoulos, et al., U.S. Pat. No. 4,868,103). A fluorophore label on the first, “donor” molecule is selected such that its emitted fluorescent energy will be absorbed by a fluorescent label on a second, “acceptor” molecule, which in turn is able to fluoresce due to the absorbed energy. Alternately, a proteinaceous “donor” molecule may simply utilize the natural fluorescent energy of tryptophan residues. Labels are chosen that emit different wavelengths of light, such that the “acceptor” molecule label may be differentiated from that of the “donor.” Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. A FET binding event can be conveniently measured through standard fluorometric detection means well known in the art (e.g., using a fluorimeter).

Another example of a fluorescence assay is fluorescence polarization (FP). For FP, only one component needs to be labeled. A binding interaction is detected by a change in molecular size of the labeled component. The size change alters the tumbling rate of the component in solution and is detected as a change in FP. See, e.g., Nasir et al. (1999) Comb Chem HTS 2:177-190; Jameson et al. (1995) Methods Enzymol 246:283; See Anal Biochem. 255:257 (1998). Fluorescence polarization can be monitored in multi-well plates. See, e.g., Parker et al. (2000) Journal of Biomolecular Screening 5:77-88; and Shoeman, et al. (1999) 38, 16802-16809.

In another embodiment, determining the ability of the target enzyme to bind to a target molecule can be accomplished using real-time Biomolecular Interaction Analysis (BIA) (See, e.g., Sjolander, S, and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target enzyme is anchored onto a solid phase. The target enzyme/test compound complexes anchored on the solid phase can be detected at the end of the reaction, e.g., the binding reaction. For example, the target enzyme can be anchored onto a solid surface, and the test compound (which is not anchored), can be labeled, either directly or indirectly, with detectable labels discussed herein.

It may be desirable to immobilize either the target enzyme or an anti-target enzyme antibody to facilitate separation of complexed from uncomplexed forms of one or both of the proteins, as well as to accommodate automation of the assay. Binding of a test compound to target enzyme, or interaction of a target enzyme with a second component in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/target enzyme fusion proteins can be adsorbed onto glutathione sepharose beads (Sigma Chemical, St. Louis, Mo., USA) or glutathione derivatized microtiter plates, which are then combined with the test compound or the test compound and either the non-adsorbed target enzyme, and the mixture incubated under conditions conducive to complex formation (e.g., at physiological conditions for salt and pH). Following incubation, the beads or microtiter plate wells are washed to remove any unbound components, the matrix immobilized in the case of beads, and the complex determined either directly or indirectly, for example, as described above. Alternatively, the complexes can be dissociated from the matrix, and the level of target enzyme binding or activity is determined using standard techniques.

Other techniques for immobilizing either a target enzyme or a test compound on matrices include using conjugation of biotin and streptavidin. Biotinylated target enzyme or test compounds can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface, e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with a target enzyme but which do not interfere with binding of the target enzyme to the test compound and/or substrate. Such antibodies can be derivatized to the wells of the plate, and unbound target enzyme trapped in the wells by antibody conjugation. Methods for detecting such complexes, in addition to those described above for the GST-immobilized complexes, include immunodetection of complexes using antibodies reactive with the target enzyme, as well as enzyme-linked assays which rely on detecting an enzymatic activity associated with the target enzyme.

Alternatively, cell free assays can be conducted in a liquid phase. In such an assay, the reaction products are separated from unreacted components, by any of a number of standard techniques, including but not limited to: differential centrifugation (See, for example, Rivas, G., and Minton, A. P., (1993) Trends Biochem Sci 18:284-7); chromatography (gel filtration chromatography, ion-exchange chromatography); electrophoresis (See, e.g., Ausubel, F. et al., eds. Current Protocols in Molecular Biology 1999, J. Wiley: New York); and immunoprecipitation (See, for example, Ausubel, F. et al., eds. (1999) Current Protocols in Molecular Biology, J. Wiley: New York). Such resins and chromatographic techniques are known to one skilled in the art (See, e.g., Heegaard, N. H., (1998) J Mol Recognit 11:141-8; Hage, D. S., and Tweed, S. A. (1997) J Chromatogr B Biomed Sci Appl. 699:499-525). Further, fluorescence energy transfer may also be conveniently utilized, as described herein, to detect binding without further purification of the complex from solution.

In a preferred embodiment, the assay includes contacting the target enzyme or biologically active portion thereof with a known compound which binds the target enzyme to form an assay mixture, contacting the assay mixture with a test compound, and determining the ability of the test compound to interact with the target enzyme, wherein determining the ability of the test compound to interact with the target enzyme includes determining the ability of the test compound to preferentially bind to the target enzyme, or to modulate the activity of the target enzyme, as compared to the known compound (e.g., a competition assay). In another embodiment, the ability of a test compound to bind to and modulate the activity of the target enzyme is compared to that of a known activator or inhibitor of such target enzyme.

The target enzymes of the invention can, in vivo, interact with one or more cellular or extracellular macromolecules, such as proteins, which are either heterologous to the host cell or endogenous to the host cell, and which may or may not be recombinantly expressed. For the purposes of this discussion, such cellular and extracellular macromolecules are referred to herein as “binding partners.” Compounds that disrupt such interactions can be useful in regulating the activity of the target enzyme. Such compounds can include, but are not limited to molecules such as antibodies, peptides, and small molecules. In an alternative embodiment, the invention provides methods for determining the ability of the test compound to modulate the activity of a target enzyme through modulation of the activity of a downstream effector of such target enzyme. For example, the activity of the effector molecule on an appropriate target can be determined, or the binding of the effector to an appropriate target can be determined, as previously described.

To identify compounds that interfere with the interaction between the target enzyme and its cellular or extracellular binding partner(s), a reaction mixture containing the target enzyme and the binding partner is prepared, under conditions and for a time sufficient, to allow the two products to form a complex. In order to test an inhibitory compound, the reaction mixture is provided in the presence and absence of the test compound. The test compound can be initially included in the reaction mixture, or can be added at a time subsequent to the addition of the target and its cellular or extracellular binding partner. Control reaction mixtures are incubated without the test compound or with a placebo. The formation of any complexes between the target product and the cellular or extracellular binding partner is then detected. The formation of a complex in the control reaction, but not in the reaction mixture containing the test compound, indicates that the compound interferes with the interaction of the target product and the interactive binding partner. Additionally, complex formation within reaction mixtures containing the test compound and normal target enzyme can also be compared to complex formation within reaction mixtures containing the test compound and mutant target enzyme. This comparison can be important in those cases wherein it is desirable to identify compounds that disrupt interactions of mutant but not normal target enzymes.

The assays described herein can be conducted in a heterogeneous or homogeneous format. Heterogeneous assays involve anchoring either the target enzyme or the binding partner, substrate, or tests compound onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target enzyme and a binding partners or substrate, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target enzyme or the interactive cellular or extracellular binding partner or substrate, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target enzyme and the interactive cellular or extracellular binding partner product or substrate is prepared in that either the target enzyme or their binding partners or substrates are labeled, but the signal generated by the label is quenched due to complex formation (See, e.g., U.S. Pat. No. 4,109,496 that utilizes this approach for immunoassays). The addition of a test substance that competes with and displaces one of the species from the preformed complex will result in the generation of a signal above background. In this way, test compounds that disrupt target enzyme-binding partner or substrate contact can be identified.

In yet another aspect, the target enzyme can be used as “bait protein” in a two-hybrid assay or three-hybrid assay (See, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent, International patent application Publication No. WO94/10300), to identify other proteins that bind to or interact with target enzyme (“target enzyme binding protein” or “target enzyme—bp”) and are involved in target enzyme pathway activity. Such target enzyme-bps can be activators or inhibitors of the target enzyme or target enzyme targets as, for example, downstream elements of the target enzyme pathway.

In another embodiment, modulators of a target enzyme's gene expression are identified. For example, a cell or cell free mixture is contacted with a candidate compound and the expression of the target enzyme mRNA or protein evaluated relative to the level of expression of target enzyme mRNA or protein in the absence of the candidate compound. When expression of the target enzyme component mRNA or protein is greater in the presence of the candidate compound than in its absence, the candidate compound is identified as a stimulator of target enzyme mRNA or protein expression. Alternatively, when expression of the target enzyme mRNA or protein is less (statistically significantly less) in the presence of the candidate compound than in its absence, the candidate compound is identified as an inhibitor of the target enzyme mRNA or protein expression. The level of the target enzyme mRNA or protein expression can be determined by methods for detecting target enzyme mRNA or protein, e.g., Westerns, Northerns, PCR, mass spectroscopy, 2-D gel electrophoresis, and so forth, all which are known to those of ordinary skill in the art.

4.1 Compounds

A compound of interest can be tested for its ability to modulate the activity of mTOR. Once such compounds are identified as having mTOR-modulating activity, they can be further tested for their antiviral activity as described herein. Alternatively, compounds can be screened for antiviral activity and optionally characterized using the mTOR screening assays described herein.

In addition, compounds that are identified as having mTOR-modulating activity can be further tested for selectivity by testing against a panel of

In one embodiment, high throughput screening methods are used to provide a combinatorial chemical or peptide library (e.g., a publicly available library) containing a large number of potential therapeutic compounds (potential modulators or ligand compounds). Such “combinatorial chemical libraries” or “ligand libraries” are then screened in one or more assays, as described herein, to identify those library members (particular chemical species or subclasses) that display a desired characteristic activity (e.g., inhibition of mTOR activity). The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (See, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (See Ausubel, Berger and Sambrook, all supra), peptide nucleic acid libraries (See, e.g., U.S. Pat. No. 5,539,083), antibody libraries (See, e.g., Vaughn et al., Nature Biotechnology, 14(3):309-314 (1996) and PCT/US96/10287), carbohydrate libraries (See, e.g., Liang et al., Science, 274:1520-1522 (1996) and International Patent Application Publication NO. WO 1997/000271), small organic molecule libraries (See, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like). Additional examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Some exemplary libraries are used to generate variants from a particular lead compound. One method includes generating a combinatorial library in which one or more functional groups of the lead compound are varied, e.g., by derivatization. Thus, the combinatorial library can include a class of compounds which have a common structural feature (e.g., scaffold or framework). Devices for the preparation of combinatorial libraries are commercially available (See, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (See, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.). The test compounds can also be obtained from: biological libraries; peptoid libraries (libraries of molecules having the functionalities of peptides, but with a novel, non-peptide backbone which are resistant to enzymatic degradation but which nevertheless remain bioactive; See, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological libraries include libraries of nucleic acids and libraries of proteins. Some nucleic acid libraries encode a diverse set of proteins (e.g., natural and artificial proteins; others provide, for example, functional RNA and DNA molecules such as nucleic acid aptamers or ribozymes. A peptoid library can be made to include structures similar to a peptide library. (See also Lam (1997) Anticancer Drug Des. 12:145). A library of proteins may be produced by an expression library or a display library (e.g., a phage display library). Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.). Enzymes can be screened for identifying compounds which can be selected from a combinatorial chemical library or any other suitable source (Hogan, Jr., Nat. Biotechnology 15:328, 1997).

Any assay herein, e.g., an in vitro assay or an in vivo assay, can be performed individually, e.g., just with the test compound, or with appropriate controls. For example, a parallel assay without the test compound, or other parallel assays without other reaction components, e.g., without a target or without a substrate. Alternatively, it is possible to compare assay results to a reference, e.g., a reference value, e.g., obtained from the literature, a prior assay, and so forth. Appropriate correlations and art known statistical methods can be used to evaluate an assay result.

Once a compound is identified as having a desired effect, production quantities of the compound can be synthesized, e.g., producing at least 50 mg, 500 mg, 5 g, or 500 g of the compound. Although a compound that is able to penetrate a host cell is preferable in the practice of the invention, a compound may be combined with solubilizing agents or administered in combination with another compound or compounds to maintain its solubility, or help it enter a host cell, e.g., by mixture with lipids. The compound can be formulated, e.g., for administration to a subject, and may also be administered to the subject.

### 5. Characterization of Antiviral Activity of Compounds

5.1 Viruses

The present invention provides compounds for use in the prevention, management and/or treatment of viral infection. The antiviral activity of compounds against any virus can be tested using techniques described herein below.

In one embodiment, the virus is a Herpesvirus (Herpesviridae). Herpesvirus include herpes simplex virus (HSV) types 1 and 2, varicella-zoster virus, human cytomegalovirus (HCMV), Epstein-Barr virus (EBV), human herpesvirus 6 (variants A and B), human herpesvirus 7, human herpesvirus 8 (Kaposi's sarcoma—associated herpes virus, KSHV), and cercopithecine herpesvirus 1 (B virus). B virus is a monkey virus that can occasionally infect humans. Human herpesvirus are listed in Table 2.

TABLE 2
The Human Herpesvirus

| Subgroup | Virus |
|---|---|
| alpha | Herpes simplex virus type 1 (human herpesvirus 1) |
| alpha | Herpes simplex virus type 2 (human herpesvirus 2) |
| alpha | Varicella zoster virus (human herpesvirus 3) |
| beta | Cytomegalovirus (human herpesvirus 5) |
| beta | Human herpesvirus 6 |
| beta | Human herpesvirus 7 |
| gamma | Epstein-Barr virus (human herpesvirus 4) |
| gamma | Kaposi Sarcoma-associated herpesvirus |
| | (human herpesvirus 8) |

In specific embodiments, the virus infects humans. In other embodiments, the virus infects non-human animals. In a specific embodiment, the virus infects pigs, fowl, other livestock, or pets.

The antiviral activities of compounds against any type, subtype or strain of virus can be assessed. For example, the antiviral activity of compounds against naturally occurring strains, variants or mutants, mutagenized viruses, reassortants and/or genetically engineered viruses can be assessed.

In some embodiments, the virus achieves peak titer in cell culture or a subject in 4 hours or less, 6 hours or less, 8 hours or less, 12 hours or less, 16 hours or less, or 24 hours or less. In other embodiments, the virus achieves peak titers in cell culture or a subject in 48 hours or less, 72 hours or less, or 1 week or less. In other embodiments, the virus achieves peak titers after more than 1 week. In accordance with these embodiments, the viral titer may be measured in the infected tissue or serum.

In some embodiments, the virus achieves in cell culture a viral titer of 10⁴ pfu/ml or more, 5×10⁴ pfu/ml or more, 10⁵ pfu/ml or more, 5×10⁵ pfu/ml or more, 10⁶ pfu/ml or more, 5×10⁶ pfu/ml or more, 10⁷ pfu/ml or more, 5×10⁷ pfu/ml or more, 10⁸ pfu/ml or more, 5×10⁸ pfu/ml or more, 10⁹ pfu/ml or more, 5×10⁹ pfu/ml or more, or 10¹⁰ pfu/ml or more. In certain embodiments, the virus achieves in cell culture a viral titer of 10⁴ pfu/ml or more, 5×10⁴ pfu/ml or more, 10⁵ pfu/ml or more, 5×10⁵ pfu/ml or more, 10⁶ pfu/ml or more, 5×10⁶ pfu/ml or more, 10⁷ pfu/ml or more, 5×10⁷ pfu/ml or more, 10⁸ pfu/ml or more, 5×10⁸ pfu/ml or more, 10⁹ pfu/ml or more, 5×10⁹ pfu/ml or more, or 10¹⁰ pfu/ml or more within 4 hours, 6 hours, 8 hours, 12 hours, 16 hours, or 24 hours or less. In other embodiments, the virus achieves in cell culture a viral titer of 10⁴ pfu/ml or more, 5×10⁴ pfu/ml or more, 10⁵ pfu/ml or more, 5×10⁵ pfu/ml or more, 10⁶ pfu/ml or more, 5×10⁶ pfu/ml or more, 10⁷ pfu/ml or more, 5×10⁷ pfu/ml or more, 10⁸ pfu/ml or more, 5×10⁸ pfu/ml or more, 10⁹ pfu/ml or more, 5×10⁹ pfu/ml or more, or 10¹⁰ pfu/ml or more within 48 hours, 72 hours, or 1 week.

In some embodiments, the virus achieves a viral yield of 1 pfu/ml or more, 10 pfu/ml or more, 5×10¹ pfu/ml or more, 10² pfu/ml or more, 5×10² pfu/ml or more, 10³ pfu/ml or more, 2.5×10³ pfu/ml or more, 5×10³ pfu/ml or more, 10⁴ pfu/ml or more, 2.5×10⁴ pfu/ml or more, 5×10⁴ pfu/ml or more, or 10⁵ pfu/ml or more in a subject. In certain embodiments, the virus achieves a viral yield of 1 pfu/ml or more, 10 pfu/ml or more, 5×10¹ pfu/ml or more, 10² pfu/ml or more, 5×10² pfu/ml or more, 10³ pfu/ml or more, 2.5×10³ pfu/ml or more, 5×10³ pfu/ml or more, 10⁴ pfu/ml or more, 2.5×10⁴ pfu/ml or more, 5×10⁴ pfu/ml or more, or 10⁵ pfu/ml or more in a subject within 4 hours, 6 hours, 8 hours, 12 hours, 16 hours, 24 hours, or 48 hours. In certain embodiments, the virus achieves a viral yield of 1 pfu/ml or more, 10 pfu/ml or more, 10¹ pfu/ml or more, 5×10¹ pfu/ml or more, 10² pfu/ml or more, 5×10² pfu/ml or more, 10³ pfu/ml or more, 2.5×10³ pfu/ml or more, 5×10³ pfu/ml or more, 10⁴ pfu/ml or more, 2.5×10⁴ pfu/ml or more, 5×10⁴ pfu/ml or more, or 10⁵ pfu/ml or more in a subject within 48 hours, 72 hours, or 1 week. In accordance with these embodiments, the viral yield may be measured in the infected tissue or serum. In a specific embodiment, the subject is immunocompetent. In another embodiment, the subject is immunocompromised or immunosuppressed.

In some embodiments, the virus achieves a viral yield of 1 pfu or more, 10 pfu or more, 5×10¹ pfu or more, 10² pfu or more, 5×10² pfu or more, 10³ pfu or more, 2.5×10³ pfu or more, 5×10³ pfu or more, 10⁴ pfu or more, 2.5×10⁴ pfu or more, 5×10⁴ pfu or more, or 10⁵ pfu or more in a subject. In certain embodiments, the virus achieves a viral yield of 1 pfu or more, 10 pfu or more, 5×10¹ pfu or more, 10² pfu or more, 5×10² pfu or more, 10³ pfu or more, 2.5×10³ pfu or more, 5×10³ pfu or more, 10⁴ pfu or more, 2.5×10⁴ pfu or more, 5×10⁴ pfu or more, or 10⁵ pfu or more in a subject within 4 hours, 6 hours, 8 hours, 12 hours, 16 hours, 24 hours, or 48 hours. In certain embodiments, the virus achieves a viral yield of 1 pfu or more, 10 pfu or more, 10¹ pfu or more, 5×10¹ pfu or more, 10² pfu or more, 5×10² pfu or more, 10³ pfu or more, 2.5×10³ pfu or more, 5×10³ pfu or more, 10⁴ pfu or more, 2.5×10⁴ pfu or more, 5×10⁴ pfu or more, or 10⁵ pfu or more in a subject within 48 hours, 72 hours, or 1 week. In accordance with these embodiments, the viral yield may be measured in the infected tissue or serum. In a specific embodiment, the subject is immunocompetent. In another embodiment, the subject is immunocompromised or immunosuppressed.

In some embodiments, the virus achieves a viral yield of 1 infectious unit or more, 10 infectious units or more, 5×10¹ infectious units or more, 10² infectious units or more, 5×10² infectious units or more, 10³ infectious units or more, 2.5×10³ infectious units or more, 5×10³ infectious units or more, 10⁴ infectious units or more, 2.5×10⁴ infectious units or more, 5×10⁴ infectious units or more, or 10⁵ infectious units or more in a subject. In certain embodiments, the virus achieves a viral yield of 1 infectious unit or more, 10 infectious units or more, 5×10¹ infectious units or more, 10² infectious units or more, 5×10² infectious units or more, 10³ infectious units or more, 2.5×10³ infectious units or more, 5×10³ infectious units or more, 10⁴ infectious units or more, 2.5×10⁴ infectious units or more, 5×10⁴ infectious units or more, or 10⁵ infectious units or more in a subject within 4 hours, 6 hours, 8 hours, 12 hours, 16 hours, 24 hours, or 48 hours. In certain embodiments, the virus achieves a viral yield of 1 infectious unit or more, 10 infectious units or more, 10¹ infectious units or more, 5×10¹ infectious units or more, 10² infectious units or more, 5×10² infectious units or more, 10³ infectious units or more, 2.5×10³ infectious units or more, 5×10³ infectious units or more, 10⁴ infectious units or more, 2.5×10⁴ infectious units or more, 5×10⁴ infectious units or more, or 10⁵ infectious units or more in a subject within 48 hours, 72 hours, or 1 week. In accordance with these embodiments, the viral yield may be measured in the infected tissue or serum. In a specific embodiment, the subject is immunocompetent. In another embodiment, the subject is immunocompromised or immunosuppressed. In a specific embodiment, the virus achieves a yield of less than 10⁴ infectious units. In other embodiments the virus achieves a yield of 10⁵ or more infectious units.

In some embodiments, the virus achieves a viral titer of 1 infectious unit per ml or more, 10 infectious units per ml or more, 5×10¹ infectious units per ml or more, 10² infectious units per ml or more, 5×10² infectious units per ml or more, 10³ infectious units per ml or more, 2.5×10³ infectious units per ml or more, 5×10³ infectious units per ml or more, 10⁴ infectious units per ml or more, 2.5×10⁴ infectious units per ml or more, 5×10⁴ infectious units per ml or more, or 10⁵ infectious units per ml or more in a subject. In certain embodiments, the virus achieves a viral titer of 10 infectious units per ml or more, 5×10¹ infectious units per ml or more, 10² infectious units per ml or more, 5×10² infectious units per ml or more, 10³ infectious units per ml or more, 2.5×10³ infectious units per ml or more, 5×10³ infectious units per ml or more, 10⁴ infectious units per ml or more, 2.5×10⁴ infectious units per ml or more, 5×10⁴ infectious units per ml or more, or 10⁵ infectious units per ml or more in a subject within 4 hours, 6 hours, 8 hours, 12 hours, 16 hours, 24 hours, or 48 hours. In certain embodiments, the virus achieves a viral titer of 1 infectious unit per mL or more, 10 infectious units per ml or more, 5×10¹ infectious units per ml or more, 10² infectious units per ml or more, 5×10² infectious units per ml or more, 10³ infectious units per mL or more, 2.5×10³ infectious units per ml or more, 5×10³ infectious units per ml or more, 10⁴ infectious units per ml or more, 2.5×10⁴ infectious units per ml or more, 5×10⁴ infectious units per ml or more, or 10⁵ infectious units per ml or more in a subject within 48 hours, 72 hours, or 1 week. In accordance with these embodiments, the viral titer may be measured in the infected tissue or serum. In a specific embodiment, the subject is immunocompetent. In another embodiment, the subject is immunocompromised or immunosuppressed. In a specific embodiment, the virus achieves a titer of less than 10⁴ infectious units per ml. In some embodiments, the virus achieves 10⁵ or more infectious units per ml.

In some embodiments, the virus infects a cell and produces, 10¹ or more, 2.5×10¹ or more, 5×10¹ or more, 7.5×10¹ or more, 10² or more, 2.5×10² or more, 5×10² or more, 7.5×10² or more, 10³ or more, 2.5×10³ or more, 5×10³ or more, 7.5×10³ or more, 10⁴ or more, 2.5×10⁴ or more, 5×10⁴ or more, 7.5×10⁴ or more, or 10⁵ or more viral particles per cell. In certain embodiments, the virus infects a cell and produces 10 or more, 10¹ or more, 2.5×10¹ or more, 5×10¹ or more, 7.5×10¹ or more, 10² or more, 2.5×10² or more, 5×10² or more, 7.5×10² or more, 10³ or more, 2.5×10³ or more, 5×10³ or more, 7.5×10³ or more, 10⁴ or more, 2.5×10⁴ or more, 5×10⁴ or more, 7.5×10⁴ or more, or 10⁵ or more viral particles per cell within 4 hours, 6 hours, 8 hours, 12 hours, 16 hours, or 24 hours. In other embodiments, the virus infects a cell and produces 10 or more, 10¹ or more, 2.5×10¹ or more, 5×10¹ or more, 7.5×10¹ or more, 10² or more, 2.5×10² or more, 5×10² or more, 7.5×10² or more, 10³ or more, 2.5×10³ or more, 5×10³ or more, 7.5×10³ or more, 10⁴ or more, 2.5×10⁴ or more, 5×10⁴ or more, 7.5×10⁴ or more, or 10⁵ or more viral particles per cell within 48 hours, 72 hours, or 1 week.

In other embodiments, the virus is latent for a period of about at least 1 day, 2 days, 3 days, 4 days, 5 days, 6 days, 7 days, 8 days, 9 days, 10 days, 11 days, 12 days, 13 days, 14 days, or 15 days. In another embodiment, the virus is latent for a period of about at least 1 week, or 2 weeks, 3 weeks, 4 weeks, 5 weeks, 6 weeks, 7 weeks, 8 weeks, 9 weeks, or 10 weeks. In a further embodiment, the virus is latent for a period of about at least 1 month, 2 months, 3 months, 4 months, 5 months, 6 months, 7 months, 8 months, 9 months, 10 months, or 11 months. In yet another embodiment, the virus is latent for a period of about at least 1 year, 2 years, 3 years, 4 years, 5 years, 6 years, 7 years, 8 years, 9 years, 10 years, 11 years, 12 years, 13 years, 14 years, or 15 years. In some embodiments, the virus is latent for a period of greater than 15 years.

5.2 In vitro Assays to Detect Antiviral Activity

The antiviral activity of compounds may be assessed in various in vitro assays described herein or others known to one of skill in the art. Non-limiting examples of the viruses that can be tested for compounds with antiviral activities against such viruses are provided herein. In specific embodiments, compounds exhibit an activity profile that is consistent with their ability to inhibit viral replication while maintaining low toxicity with respect to eukaryotic cells, preferably mammalian cells. For example, the effect of a compound on the replication of a virus may be determined by infecting cells with different dilutions of a virus in the presence or absence of various dilutions of a compound, and assessing the effect of the compound on, e.g., viral replication, viral genome replication, and/or the synthesis of viral proteins. Alternatively, the effect of a compound on the replication of a virus may be determined by contacting cells with various dilutions of a compound or a placebo, infecting the cells with different dilutions of a virus, and assessing the effect of the compound on, e.g., viral replication, viral genome replication, and/or the synthesis of viral proteins. Altered viral replication can be assessed by, e.g., plaque formation. The production of viral proteins can be assessed by, e.g., ELISA, Western blot, immunofluorescence, or flow cytometry analysis. The production of viral nucleic acids can be assessed by, e.g., RT-PCR, PCR, Northern blot analysis, or Southern blot.

In certain embodiments, compounds reduce the replication of a virus by approximately 10%, preferably 15%, 25%, 30%, 45%, 50%, 60%, 75%, 95% or more relative to a negative control (e.g., PBS, DMSO) in an assay described herein or others known to one of skill in the art. In some embodiments, compounds reduce the replication of a virus by about at least 1.5 fold, 2, fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 75 fold, 100 fold, 500 fold, or 1000 fold relative to a negative control (e.g., PBS, DMSO) in an assay described herein or others known to one of skill in the art. In other embodiments, compounds reduce the replication of a virus by about at least 1.5 to 3 fold, 2 to 4 fold, 3 to 5 fold, 4 to 8 fold, 6 to 9 fold, 8 to 10 fold, 2 to 10 fold, 5 to 20 fold, 10 to 40 fold, 10 to 50 fold, 25 to 50 fold, 50 to 100 fold, 75 to 100 fold, 100 to 500 fold, 500 to 1000 fold, or 10 to 1000 fold relative to a negative control (e.g., PBS, DMSO) in an assay described herein or others known to one of skill in the art. In other embodiments, compounds reduce the replication of a virus by about 1 log, 1.5 logs, 2 logs, 2.5 logs, 3 logs, 3.5 logs, 4 logs, 4.5 logs, 5 logs or more relative to a negative control (e.g., PBS, DMSO) in an assay described herein or others known to one of skill in the art. In accordance with these embodiments, such compounds may be further assessed for their safety and efficacy in assays such as those described herein.

In certain embodiments, compounds reduce the replication of a viral genome by approximately 10%, preferably 15%, 25%, 30%, 45%, 50%, 60%, 75%, 95% or more relative to a negative control (e.g., PBS, DMSO) in an assay described herein or others known to one of skill in the art. In some embodiments, compounds reduce the replication of a viral genome by about at least 1.5 fold, 2, fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 75 fold, 100 fold, 500 fold, or 1000 fold relative to a negative control (e.g., PBS, DMSO) in an assay described herein or others known to one of skill in the art. In other embodiments, compounds reduce the replication of a viral genome by about at least 1.5 to 3 fold, 2 to 4 fold, 3 to 5 fold, 4 to 8 fold, 6 to 9 fold, 8 to 10 fold, 2 to 10 fold, 5 to 20 fold, 10 to 40 fold, 10 to 50 fold, 25 to 50 fold, 50 to 100 fold, 75 to 100 fold, 100 to 500 fold, 500 to 1000 fold, or 10 to 1000 fold relative to a negative control (e.g., PBS, DMSO) in an assay described herein or others known to one of skill in the art. In other embodiments, compounds reduce the replication of a viral genome by about 1 log, 1.5 logs, 2 logs, 2.5 logs, 3 logs, 3.5 logs, 4 logs, 4.5 logs, 5 logs or more relative to a negative control (e.g., PBS, DMSO) in an assay described herein or others known to one of skill in the art. In accordance with these embodiments, such compounds may be further assessed for their safety and efficacy in assays such as those described herein.

In certain embodiments, compounds reduce the synthesis of viral proteins by approximately 10%, preferably 15%, 25%, 30%, 45%, 50%, 60%, 75%, 95% or more relative to a negative control (e.g., PBS, DMSO) in an assay described herein or others known to one of skill in the art. In some embodiments, compounds reduce the synthesis of viral proteins by approximately at least 1.5 fold, 2, fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 75 fold, 100 fold, 500 fold, or 1000 fold relative to a negative control (e.g., PBS, DMSO) in an assay described herein or others known to one of skill in the art. In other embodiments, compounds reduce the synthesis of viral proteins by approximately at least 1.5 to 3 fold, 2 to 4 fold, 3 to 5 fold, 4 to 8 fold, 6 to 9 fold, 8 to 10 fold, 2 to 10 fold, 5 to 20 fold, 10 to 40 fold, 10 to 50 fold, 25 to 50 fold, 50 to 100 fold, 75 to 100 fold, 100 to 500 fold, 500 to 1000 fold, or 10 to 1000 fold relative to a negative control (e.g., PBS, DMSO) in an assay described herein or others known to one of skill in the art. In other embodiments, compounds reduce the synthesis of viral proteins by approximately 1 log, 1.5 logs, 2 logs, 2.5 logs, 3 logs, 3.5 logs, 4 logs, 4.5 logs, 5 logs or more relative to a negative control (e.g., PBS, DMSO) in an assay described herein or others known to one of skill in the art. In accordance with these embodiments, such compounds may be further assessed for their safety and efficacy in assays such as those described herein.

In some embodiments, compounds result in about a 1.5 fold or more, 2 fold or more, 3 fold or more, 4 fold or more, 5 fold or more, 6 fold or more, 7 fold or more, 8 fold or more, 9 fold or more, 10 fold or more, 15 fold or more, 20 fold or more, 25 fold or more, 30 fold or more, 35 fold or more, 40 fold or more, 45 fold or more, 50 fold or more, 60 fold or more, 70 fold or more, 80 fold or more, 90 fold or more, or 100 fold or more inhibition/reduction of viral yield per round of viral replication. In certain embodiments, compounds result in about a 2 fold or more reduction inhibition/reduction of viral yield per round of viral replication. In specific embodiments, compounds result in about a 10 fold or more inhibition/reduction of viral yield per round of viral replication.

The in vitro antiviral assays can be conducted using any eukaryotic cell, including primary cells and established cell lines. The cell or cell lines selected should be susceptible to infection by a virus of interest. Non-limiting examples of mammalian cell lines that can be used in standard in vitro antiviral assays (e.g., viral cytopathic effect assays, neutral red update assays, viral yield assay, plaque reduction assays) for the respective viruses are set out in Table 3.

TABLE 3
Examples of Mammalian Cell Lines in Antiviral Assays

| Virus | Cell Line |
|---|---|
| herpes simplex virus | primary human fibroblasts (MRC-5 cells) |
| (HSV) | Vero cells |
| human cytomegalovirus | Primary human fibroblasts (MRC-5 cells) |
| (HCMV) | |
| hepatitis C virus | Huh7 (or Huh7.7) |
| | primary human hepatocytes (PHH) |
| | immortalized human hepatocytes (IHH) |
| HHV-6 | Human Cord Blood Lymphocytes (CBL) |
| | Human T cell lymphoblastoid cell lines |
| | (HSB-2 and SupT-1) |
| HHV-8 | Human B-cell lymphoma cell line (BCBL-1) |
| EBV | Human umbilical cord blood lymphocytes |

Sections 5.2.1 to 5.2.7 below provide non-limiting examples of antiviral assays that can be used to characterize the antiviral activity of compounds against the respective virus. One of skill in the art will know how to adapt the methods described in Sections 5.2.1 to 5.2.7 to other viruses by, e.g., changing the cell system and viral pathogen, such as described in Table 3.

5.2.1 Viral Cytopathic Effect (CPE) Assay

CPE is the morphological changes that cultured cells undergo upon being infected by most viruses. These morphological changes can be observed easily in unfixed, unstained cells by microscopy. Forms of CPE, which can vary depending on the virus, include, but are not limited to, rounding of the cells, appearance of inclusion bodies in the nucleus and/or cytoplasm of infected cells, and formation of syncytia, or polykaryocytes (large cytoplasmic masses that contain many nuclei). For adenovirus infection, crystalline arrays of adenovirus capsids accumulate in the nucleus to form an inclusion body.

The CPE assay can provide a measure of the antiviral effect of a compound. In a non-limiting example of such an assay, compounds are serially diluted (e.g. 1000, 500, 100, 50, 10, 1 μg/ml) and added to 3 wells containing a cell monolayer (preferably mammalian cells at 80-100% confluent) of a 96-well plate. Within 5 minutes, viruses are added and the plate sealed, incubated at 37° C. for the standard time period required to induce near-maximal viral CPE (e.g., approximately 48 to 120 hours, depending on the virus and multiplicity of infection). CPE is read microscopically after a known positive control drug is evaluated in parallel with compounds in each test. The data are expressed as 50% effective concentrations or approximated virus-inhibitory concentration, 50% endpoint (EC50) and cell-inhibitory concentration, 50% endpoint (IC50). General selectivity index (“SI”) is calculated as the IC50 divided by the EC50. These values can be calculated using any method known in the art, e.g., the computer software program MacSynergy II by M. N. Prichard, K. R. Asaltine, and C. Shipman, Jr., University of Michigan, Ann Arbor, Mich.

In one embodiment, a compound has an SI of greater than 3, or 4, or 5, or 6, or 7, or 8, or 9, or 10, or 11, or 12, or 13, or 14, or 15, or 20, or 21, or 22, or 23, or 24, or 25, or 30, or 35, or 40, or 45, or 50, or 60, or 70, or 80, or 90, or 100, or 200, or 300, or 400, or 500, 1,000, or 10,000. In some embodiments, a compound has an SI of greater than 10. In a specific embodiment, compounds with an SI of greater than 10 are further assessed in other in vitro and in vivo assays described herein or others known in the art to characterize safety and efficacy.

5.2.2 Neutral Red (NR) Dye Uptake Assay

The NR Dye Uptake assay can be used to validate the CPE inhibition assay. In a non-limiting example of such an assay, the same 96-well microplates used for the CPE inhibition assay can be used. Neutral red is added to the medium, and cells not damaged by virus take up a greater amount of dye. The percentage of uptake indicating viable cells is read on a microplate autoreader at dual wavelengths of 405 and 540 nm, with the difference taken to eliminate background. (See McManus et al., Appl. Environment. Microbiol. 31:35-38, 1976). An EC50 is determined for samples with infected cells and contacted with compounds, and an IC50 is determined for samples with uninfected cells contacted with compounds.

5.2.3 Virus Yield Assay

Lysed cells and supernatants from infected cultures such as those in the CPE inhibition assay can be used to assay for virus yield (production of viral particles after the primary infection). In a non-limiting example, these supernatants are serial diluted and added onto monolayers of susceptible cells (e.g., Vero cells). Development of CPE in these cells is an indication of the presence of infectious viruses in the supernatant. The 90% effective concentration (EC90), the test compound concentration that inhibits virus yield by 1 log₁₀, is determined from these data using known calculation methods in the art. In one embodiment, the EC90 of compound is at least 1.5 fold, 2 fold, 3 fold, 4 fold, 5 fold, 6 fold, 7 fold, 8 fold, 9 fold, 10 fold, 20 fold, 30 fold, 40 fold, or 50 fold less than the EC90 of the negative control sample.

5.2.4 Plaque Reduction Assay

In a non-limiting example of such an assay, the virus is diluted into various concentrations and added to each well containing a monolayer of the target mammalian cells in triplicate. The plates are then incubated for a period of time to achieve effective infection of the control sample (e.g., 1 hour with shaking every fifteen minutes). After the incubation period, an equal amount of 1% agarose is added to an equal volume of each compound dilution prepared in 2× concentration. In certain embodiments, final compound concentrations between 0.03 μg/ml to 100 μg/ml can be tested with a final agarose overlay concentration of 0.5%. The drug agarose mixture is applied to each well in 2 ml volume and the plates are incubated for three days, after which the cells are stained with a 1.5% solution of neutral red. At the end of the 4-6 hour incubation period, the neutral red solution is aspirated, and plaques counted using a stereomicroscope. Alternatively, a final agarose concentration of 0.4% can be used. In other embodiments, the plates are incubated for more than three days with additional overlays being applied on day four and on day 8 when appropriate. In another embodiment, the overlay medium is liquid rather than semi-solid.

5.2.5 Virus Titer Assay

In this non-limiting example, a monolayer of the target mammalian cell line is infected with different amounts (e.g., multiplicity of 3 plaque forming units (pfu) or 5 pfu) of virus (e.g., HCMV or HSV) and subsequently cultured in the presence or absence of various dilutions of compounds (e.g., 0.1 μg/ml, 1 μg/ml, 5 μg/ml, or 10 μg/ml). Infected cultures are harvested 48 hours or 72 hours post infection and titered by standard plaque assays known in the art on the appropriate target cell line (e.g., Vero cells, MRC5 cells). In certain embodiments, culturing the infected cells in the presence of compounds reduces the yield of infectious virus by at least 1.5 fold, 2, fold, 3, fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 100 fold, 500 fold, or 1000 fold relative to culturing the infected cells in the absence of compounds. In a specific embodiment, culturing the infected cells in the presence of compounds reduces the PFU/ml by at least 10 fold relative to culturing the infected cells in the absence of compounds.

In certain embodiments, culturing the infected cells in the presence of compounds reduces the yield of infectious virus by at least 0.5 log 10, 1 log 10, 1.5 log 10, 2 log 10, 2.5 log 10, 3 log 10, 3.5 log 10, 4 log 10, 4.5 log 10, 5 log 10, 5.5 log 10, 6 log 10, 6.5 log 10, 7 log 10, 7.5 log 10, 8 log 10, 8.5 log 10, or 9 log 10 relative to culturing the infected cells in the absence of compounds. In a specific embodiment, culturing the infected cells in the presence of compounds reduces the yield of infectious virus by at least 1 log 10 or 2 log 10 relative to culturing the infected cells in the absence of compounds. In another specific embodiment, culturing the infected cells in the presence of compounds reduces the yield of infectious virus by at least 2 log 10 relative to culturing the infected cells in the absence of compounds.

5.2.6 Flow Cytometry Assay

Flow cytometry can be utilized to detect expression of virus antigens in infected target cells cultured in the presence or absence of compounds (See, e.g., McSharry et al., Clinical Microbiology Rev., 1994, 7:576-604). Non-limiting examples of viral antigens that can be detected on cell surfaces by flow cytometry include, but are not limited to gB, gC, gC, and gE of HSV; gpI of varicella-zoster virus; gB of HCMV; and gp110/60 of HHV-6. In other embodiments, intracellular viral antigens or viral nucleic acid can be detected by flow cytometry with techniques known in the art.

5.2.7 Genetically Engineered Cell Lines for Antiviral Assays

Various cell lines for use in antiviral assays can be genetically engineered to render them more suitable hosts for viral infection or viral replication and more convenient substrates for rapidly detecting virus-infected cells (See, e.g., Olivo, P. D., Clin. Microbiol. Rev., 1996, 9:321-334). In some aspects, these cell lines are available for testing the antiviral activity of compound on blocking any step of viral replication, such as, transcription, translation, pregenome encapsidation, reverse transcription, particle assembly and release. Nonlimiting examples of genetically engineered cells lines for use in antiviral assays with the respective virus are discussed below.

The antiviral effect of compound can be assayed against EBV by measuring the level of viral capsid antigen (VCA) production in Daudi cells using an ELISA assay. Various concentrations of compounds are tested (e.g., 50 mg/ml to 0.03 mg/ml), and the results obtained from untreated and compound treated cells are used to calculate an EC50 value. Selected compounds that have good activity against EBV VCA production without toxicity will be tested for their ability to inhibit EBV DNA synthesis.

For assays with HSV, the BHKICP6LacZ cell line, which was stably transformed with the E. coli lacZ gene under the transcriptional control of the HSV-1 UL39 promoter, can be used (See Stabell et al., 1992, Methods 38:195-204). Infected cells are detected using β-galactosidase assays known in the art, e.g., colorimetric assay.

5.3 Characterization of Safety and Efficacy of Compounds

The safety and efficacy of compounds can be assessed using technologies known to one of skill in the art. Sections 5.4 and 5.5 below provide non-limiting examples of cytotoxicity assays and animal model assays, respectively, to characterize the safety and efficacy of compounds. In certain embodiments, the cytotoxicity assays described herein are conducted before, concurrently, or following the in vitro antiviral assays described herein.

In some embodiments, compounds differentially affect the viability of uninfected cells and cells infected with virus. The differential effect of a compound on the viability of virally infected and uninfected cells may be assessed using techniques such as those described herein, or other techniques known to one of skill in the art. In certain embodiments, compounds are more toxic to cells infected with a virus than uninfected cells. In specific embodiments, compounds preferentially affect the viability of cells infected with a virus. Without being bound by any particular concept, the differential effect of a compound on the viability of uninfected and virally infected cells may be the result of the compound targeting a particular enzyme or protein that is differentially expressed or regulated or that has differential activities in uninfected and virally infected cells. For example, viral infection and/or viral replication in an infected host cells may alter the expression, regulation, and/or activities of enzymes and/or proteins. Accordingly, in some embodiments, other compounds that target the same enzyme, protein or metabolic pathway are examined for antiviral activity. In other embodiments, congeners of compounds that differentially affect the viability of cells infected with virus are designed and examined for antiviral activity. Non-limiting examples of antiviral assays that can be used to assess the antiviral activity of compound are provided herein.

### 5.4 Cytotoxicity Studies

In a preferred embodiment, the cells are animal cells, including primary cells and cell lines. In some embodiments, the cells are human cells. In certain embodiments, cytotoxicity is assessed in one or more of the following cell lines: U937, a human monocyte cell line; primary peripheral blood mononuclear cells (PBMC); Huh7, a human hepatoblastoma cell line; 293T, a human embryonic kidney cell line; and THP-1, monocytic cells. Other non-limiting examples of cell lines that can be used to test the cytotoxicity of compounds are provided in Table 3.

Many assays well-known in the art can be used to assess viability of cells (infected or uninfected) or cell lines following exposure to a compound and, thus, determine the cytotoxicity of the compound. For example, cell proliferation can be assayed by measuring Bromodeoxyuridine (BrdU) incorporation (See, e.g., Hoshino et al., 1986, Int. J. Cancer 38, 369; Campana et al., 1988, J. Immunol. Meth. 107:79), (3H) thymidine incorporation (See, e.g., Chen, J., 1996, Oncogene 13:1395-403; Jeoung, J., 1995, J. Biol. Chem. 270:18367 73), by direct cell count, or by detecting changes in transcription, translation or activity of known genes such as proto-oncogenes (e.g., fos, myc) or cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as ELISA, Western blotting or immunoprecipitation using antibodies, including commercially available antibodies. mRNA can be quantitated using methods that are well known and routine in the art, for example, using northern analysis, RNase protection, or polymerase chain reaction in connection with reverse transcription. Cell viability can be assessed by using trypan-blue staining or other cell death or viability markers known in the art. In a specific embodiment, the level of cellular ATP is measured to determined cell viability.

In specific embodiments, cell viability is measured in three-day and seven-day periods using an assay standard in the art, such as the CellTiter-Glo Assay Kit (Promega) which measures levels of intracellular ATP. A reduction in cellular ATP is indicative of a cytotoxic effect. In another specific embodiment, cell viability can be measured in the neutral red uptake assay. In other embodiments, visual observation for morphological changes may include enlargement, granularity, cells with ragged edges, a filmy appearance, rounding, detachment from the surface of the well, or other changes. These changes are given a designation of T (100% toxic), PVH (partially toxic-very heavy-80%), PH (partially toxic-heavy-60%), P (partially toxic-40%), Ps (partially toxic-slight-20%), or 0 (no toxicity-0%), conforming to the degree of cytotoxicity seen. A 50% cell inhibitory (cytotoxic) concentration (IC50) is determined by regression analysis of these data.

Compounds can be tested for in vivo toxicity in animal models. For example, animal models, described herein and/or others known in the art, used to test the antiviral activities of compounds can also be used to determine the in vivo toxicity of these compounds. For example, animals are administered a range of concentrations of compounds. Subsequently, the animals are monitored over time for lethality, weight loss or failure to gain weight, and/or levels of serum markers that may be indicative of tissue damage (e.g., creatine phosphokinase level as an indicator of general tissue damage, level of glutamic oxalic acid transaminase or pyruvic acid transaminase as indicators for possible liver damage). These in vivo assays may also be adapted to test the toxicity of various administration mode and/or regimen in addition to dosages.

The toxicity and/or efficacy of a compound in accordance with the invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. A compound identified in accordance with the invention that exhibits large therapeutic indices is preferred. While a compound identified in accordance with the invention that exhibits toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of a compound identified in accordance with the invention for use in humans. The dosage of such agents lies preferably within a range of circulating concentrations that include the ED50 with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high-performance liquid chromatography. Additional information concerning dosage determination is provided herein.

5.5 Animal Models

Compounds and compositions are preferably assayed in vivo for the desired therapeutic or prophylactic activity prior to use in humans. For example, in vivo assays can be used to determine whether it is preferable to administer a compound and/or another therapeutic agent. For example, to assess the use of a compound to prevent a viral infection, the compound can be administered before the animal is infected with the virus. In another embodiment, a compound can be administered to the animal at the same time that the animal is infected with the virus. To assess the use of a compound to treat or manage a viral infection, in one embodiment, the compound is administered after a viral infection in the animal. In another embodiment, a compound is administered to the animal at the same time that the animal is infected with the virus to treat and/or manage the viral infection. In a specific embodiment, the compound is administered to the animal more than one time.

Compounds can be tested for antiviral activity against virus in animal models systems including, but are not limited to, rats, mice, chicken, cows, monkeys, pigs, goats, sheep, dogs, rabbits, guinea pigs, etc. In a specific embodiment of the invention, compounds are tested in a mouse model system. Such model systems are widely used and well-known to the skilled artisan.

Animals are infected with virus and concurrently or subsequently treated with a compound or placebo. Samples obtained from these animals (e.g., serum, urine, sputum, semen, saliva, plasma, or tissue sample) can be tested for viral replication via well known methods in the art, e.g., those that measure altered viral replication (as determined, e.g., by plaque formation) or the production of viral proteins (as determined, e.g., by Western blot, ELISA, or flow cytometry analysis) or viral nucleic acids (as determined, e.g., by RT-PCR, northern blot analysis or southern blot). For quantitation of virus in tissue samples, tissue samples are homogenized in phosphate-buffered saline (PBS), and dilutions of clarified homogenates are adsorbed for 1 hour at 37° C. onto monolayers of cells (e.g., Vero, CEF or MDCK cells). In other assays, histopathologic evaluations are performed after infection, preferably evaluations of the organ(s) the virus is known to target for infection. Virus immunohistochemistry can be performed using a viral-specific monoclonal antibody. Non-limiting exemplary animal models described below (Sections 5.5.1-5.5.2) can be adapted for other viral systems.

The effect of a compound on the virulence of a virus can also be determined using in vivo assays in which the titer of the virus in an infected subject administered a compound, the length of survival of an infected subject administered a compound, the immune response in an infected subject administered a compound, the number, duration and/or severity of the symptoms in an infected subject administered a compound, and/or the time period before onset of one or more symptoms in an infected subject administered a compound is assessed. Techniques known to one of skill in the art can be used to measure such effects.

5.5.1 Herpes Simplex Virus (HSV)

Mouse models of herpes simplex virus type 1 or type 2 (HSV-1 or HSV-2) can be employed to assess the antiviral activity of compounds in vivo. BALB/c mice are commonly used, but other suitable mouse strains that are susceptible can also be used. Mice are inoculated by various routes with an appropriate multiplicity of infection of HSV (e.g., 10⁵ pfu of HSV-1 strain E-377 or 4×10⁴ pfu of HSV-2 strain MS) followed by administration of compounds and placebo. For i.p. inoculation, HSV-1 replicates in the gut, liver, and spleen and spreads to the CNS. For i.n. inoculation, HSV-1 replicates in the nasaopharynx and spreads to the CNS. Any appropriate route of administration (e.g., oral, topical, systemic, nasal), frequency and dose of administration can be tested to determine the optimal dosages and treatment regimens using compounds, optionally in combination with other therapies.

In a mouse model of HSV-2 genital disease, intravaginal inoculation of female Swiss Webster mice with HSV-1 or HSV-2 is carried out, and vaginal swabs are obtained to evaluate the effect of therapy on viral replication (See, e.g., Crute et al., Nature Medicine, 2002, 8:386-391). For example, viral titers by plaque assays are determined from the vaginal swabs. A mouse model of HSV-1 using SKH-1 mice, a strain of immunocompetent hairless mice, to study cutaneous lesions is also described in the art (See, e.g., Crute et al., Nature Medicine, 2002, 8:386-391 and Bolger et al., Antiviral Res., 1997, 35:157-165). Guinea pig models of HSV have also been described, See, e.g., Chen et al., Virol. J, 2004 Nov. 23, 1:11. Statistical analysis is carried out to calculate significance (e.g., a P value of 0.05 or less).

5.5.2 HCMV

Since HCMV does not generally infect laboratory animals, mouse models of infection with murine CMV (MCMV) can be used to assay antiviral activity compounds in vivo. For example, a MCMV mouse model with BALB/c mice can be used to assay the antiviral activities of compounds in vivo when administered to infected mice (See, e.g., Kern et al., Antimicrob. Agents Chemother., 2004, 48:4745-4753). Tissue homogenates isolated from infected mice treated or untreated with compounds are tested using standard plaque assays with mouse embryonic fibroblasts (MEFs). Statistical analysis is then carried out to calculate significance (e.g., a P value of 0.05 or less).

Alternatively, human tissue (i.e., retinal tissue or fetal thymus and liver tissue) is implanted into SCID mice, and the mice are subsequently infected with HCMV, preferably at the site of the tissue graft (See, e.g., Kern et al., Antimicrob. Agents Chemother., 2004, 48:4745-4753). The pfu of HCMV used for inoculation can vary depending on the experiment and virus strain. Any appropriate routes of administration (e.g., oral, topical, systemic, nasal), frequency and dose of administration can be tested to determine the optimal dosages and treatment regimens using compounds, optionally in combination with other therapies. Implant tissue homogenates isolated from infected mice treated or untreated with compounds at various time points are tested using standard plaque assays with human foreskin fibroblasts (HFFs). Statistical analysis is then carried out to calculate significance (i.e., a P value of 0.05 or less).

Guinea pig models of CMV to study antiviral agents have also been described, See, e.g., Bourne et al., Antiviral Res., 2000, 47:103-109; Bravo et al., Antiviral Res., 2003, 60:41-49; and Bravo et al, J. Infectious Diseases, 2006, 193:591-597.

### 6. Pharmaceutical Compositions

Any compound described or incorporated by referenced herein may optionally be in the form of a composition comprising the compound.

In certain embodiments provided herein, compositions (including pharmaceutical compositions) comprise a compound and a pharmaceutically acceptable carrier, excipient, or diluent.

In other embodiments provided herein are pharmaceutical compositions comprising an effective amount of a compound and a pharmaceutically acceptable carrier, excipient, or diluent. The pharmaceutical compositions are suitable for veterinary and/or human administration.

The pharmaceutical compositions provided herein can be in any form that allows for the composition to be administered to a subject, said subject preferably being an animal, including, but not limited to a human, mammal, or non-human animal, such as a cow, horse, sheep, pig, fowl, cat, dog, mouse, rat, rabbit, guinea pig, etc., and is more preferably a mammal, and most preferably a human.

In a specific embodiment and in this context, the term “pharmaceutically acceptable carrier, excipient or diluent” means a carrier, excipient or diluent approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical compositions and dosage forms comprise one or more excipients. Suitable excipients are well-known to those skilled in the art of pharmacy, and non limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a patient and the specific active ingredients in the dosage form. The composition or single unit dosage form, if desired, can also contain minor amounts of wetting or emulsifying agents, or pH buffering agents.

Lactose free compositions can comprise excipients that are well known in the art and are listed, for example, in the U.S. Pharmacopeia (USP) SP (XXI)/NF (XVI). In general, lactose free compositions comprise an active ingredient, a binder/filler, and a lubricant in pharmaceutically compatible and pharmaceutically acceptable amounts. Preferred lactose free dosage forms comprise a compound, microcrystalline cellulose, pre gelatinized starch, and magnesium stearate.

Further provided herein are anhydrous pharmaceutical compositions and dosage forms comprising one or more compounds, since water can facilitate the degradation of some compounds. For example, the addition of water (e.g., 5%) is widely accepted in the pharmaceutical arts as a means of simulating long term storage in order to determine characteristics such as shelf life or the stability of formulations over time. See, e.g., Jens T. Carstensen, Drug Stability: Principles & Practice, 2d. Ed., Marcel Dekker, NY, N.Y., 1995, pp. 379 80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous compositions and dosage forms provided herein can be prepared using anhydrous or low moisture containing ingredients and low moisture or low humidity conditions. Compositions and dosage forms that comprise lactose and at least one compound that comprises a primary or secondary amine are preferably anhydrous if substantial contact with moisture and/or humidity during manufacturing, packaging, and/or storage is expected.

An anhydrous composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Further provided herein are compositions and dosage forms that comprise one or more agents that reduce the rate by which a compound will decompose. Such agents, which are referred to herein as “stabilizers,” include, but are not limited to, antioxidants such as ascorbic acid, pH buffers, or salt buffers.

The compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a compound preferably in purified form, together with a suitable amount of carrier so as to provide the form for proper administration to the patient. The formulation should suit the mode of administration. In a preferred embodiment, the compositions or single unit dosage forms are sterile and in suitable form for administration to a subject, preferably an animal subject, more preferably a mammalian subject, and most preferably a human subject.

Compositions provided herein are formulated to be compatible with the intended route of administration. Examples of routes of administration include, but are not limited to, parenteral, e.g., intravenous, intradermal, subcutaneous, oral (e.g., inhalation), intranasal, transdermal (topical), transmucosal, intra-synovial, ophthalmic, and rectal administration. In a specific embodiment, the composition is formulated in accordance with routine procedures as a composition adapted for intravenous, subcutaneous, intramuscular, oral, intranasal, ophthalmic, or topical administration to human beings. In a preferred embodiment, a composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non aqueous liquid suspensions, oil in water emulsions, or a water in oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use.

Generally, the ingredients of compositions provided herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Pharmaceutical compositions provided herein that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms provided herein are prepared by combining a compound in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free flowing form such as powder or granules, optionally mixed with an excipient. Molded tablets can be made by molding in a suitable machine a mixture of the powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre gelatinized starch, and mixtures thereof. The binder or filler in pharmaceutical compositions provided herein is typically present in from about 50 to about 99 weight percent of the pharmaceutical composition or dosage form.

Suitable forms of microcrystalline cellulose include, but are not limited to, the materials sold as AVICEL PH 101, AVICEL PH 103 AVICEL RC 581, AVICEL PH 105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC 581. Suitable anhydrous or low moisture excipients or additives include AVICEL PH 103™ and Starch 1500 LM.

Disintegrants are used in the compositions provided herein to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms provided herein. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms provided herein include, but are not limited to, agar, alginic acid, calcium carbonate, microcrystalline cellulose, croscarmellose sodium, crospovidone, polacrilin potassium, sodium starch glycolate, potato or tapioca starch, pre gelatinized starch, other starches, clays, other algins, other celluloses, gums, and mixtures thereof.

Lubricants that can be used in pharmaceutical compositions and dosage forms provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB O SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

A compound can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled release.

All controlled release pharmaceutical products have a common goal of improving drug therapy over that achieved by their non controlled counterparts. Ideally, the use of an optimally designed controlled release preparation in medical treatment is characterized by a minimum of drug substance being employed to cure or control the condition in a minimum amount of time. Advantages of controlled release formulations include extended activity of the drug, reduced dosage frequency, and increased patient compliance. In addition, controlled release formulations can be used to affect the time of onset of action or other characteristics, such as blood levels of the drug, and can thus affect the occurrence of side (e.g., adverse) effects.

Most controlled release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or agents.

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms provided herein are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non aqueous vehicles such as, but not limited to, corn oil, cottonseed oil, peanut oil, sesame oil, ethyl oleate, isopropyl myristate, and benzyl benzoate.

Agents that increase the solubility of one or more of the compounds provided herein can also be incorporated into the parenteral dosage forms provided herein.

Transdermal, topical, and mucosal dosage forms provided herein include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms provided herein are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane 1,3 diol, isopropyl myristate, isopropyl palmitate, mineral oil, and mixtures thereof to form lotions, tinctures, creams, emulsions, gels or ointments, which are non toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

Depending on the specific tissue to be treated, additional components may be used prior to, in conjunction with, or subsequent to treatment with a compound. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more compounds. Similarly, the polarity of a solvent carrier, its ionic strength, or tonicity can be adjusted to improve delivery. Agents such as stearates can also be added to pharmaceutical compositions or dosage forms to advantageously alter the hydrophilicity or lipophilicity of one or more compounds so as to improve delivery. In this regard, stearates can serve as a lipid vehicle for the formulation, as an emulsifying agent or surfactant, and as a delivery enhancing or penetration enhancing agent. Different salts, hydrates or solvates of the compounds can be used to further adjust the properties of the resulting composition.

In certain specific embodiments, the compositions are in oral, injectable, or transdermal dosage forms. In one specific embodiment, the compositions are in oral dosage forms. In another specific embodiment, the compositions are in the form of injectable dosage forms. In another specific embodiment, the compositions are in the form of transdermal dosage forms.

### 7. Prophylactic and Therapeutic Methods

The present invention provides methods of preventing, treating and/or managing a viral infection, said methods comprising administering to a subject in need thereof one or more compounds. In a specific embodiment, the invention provides a method of preventing, treating and/or managing a viral infection, said method comprising administering to a subject in need thereof a dose of a prophylactically or therapeutically effective amount of one or more compounds or a composition comprising a compound. A compound or a composition comprising a compound may be used as any line of therapy (e.g., a first, second, third, fourth or fifth line therapy) for a viral infection.

In another embodiment, the invention relates to a method for reversing or redirecting metabolic flux altered by viral infection in a human subject by administering to a human subject in need thereof, an effective amount of one or more compounds or a composition comprising one or more compounds. For example, viral infection can be treated using combinations of the enzyme inhibition compounds that produce beneficial results, e.g., synergistic effect; reduction of side effects; a higher therapeutic index.

In specific embodiments, a compound is the only active ingredient administered to prevent, treat, manage or ameliorate said viral infection. In a certain embodiment, a composition comprising a compound is the only active ingredient.

The present invention encompasses methods for preventing, treating, and/or managing a viral infection for which no antiviral therapy is available. The present invention also encompasses methods for preventing, treating, and/or managing a viral infection as an alternative to other conventional therapies.

The present invention also provides methods of preventing, treating and/or managing a viral infection, said methods comprising administering to a subject in need thereof one or more of the compounds and one or more other therapies (e.g., prophylactic or therapeutic agents). In a specific embodiment, the other therapies are currently being used, have been used or are known to be useful in the prevention, treatment and/or management of a viral infection. Non-limiting examples of such therapies are provided herein. In a specific embodiment, one or more compounds are administered to a subject in combination with one or more of the therapies described herein. In another embodiment, one or more compounds are administered to a subject in combination with a supportive therapy, a pain relief therapy, or other therapy that does not have antiviral activity.

The combination therapies of the invention can be administered sequentially or concurrently. In one embodiment the combination therapies of the invention comprise a compound that is an mTOR inhibitor and a compound that inhibits the UPR. In one embodiment the combination therapies of the invention comprise a compound that inhibits a rapamycin-resistant function of mTOR and a compound that inhibits UPR. In one embodiment the combination therapies of the invention comprise a compound that inhibits a rapamycin-resistant function of mTOR and a compound that is a molecular chaperone. In one embodiment, the combination therapies of the invention comprise a compound and at least one other therapy which has the same mechanism of action. In another embodiment, the combination therapies of the invention comprise a compound and at least one other therapy which has a different mechanism of action than the compound.

In a specific embodiment, the combination therapies of the present invention improve the prophylactic and/or therapeutic effect of a compound by functioning together with the compound to have an additive or synergistic effect. In another embodiment, the combination therapies of the present invention reduce the side effects associated with each therapy taken alone.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

7.1 Patient Population

According to the invention, compounds, compositions comprising a compound, or a combination therapy are administered to a subject suffering from a viral infection. In other embodiments, compounds, compositions comprising a compound, or a combination therapy are administered to a subject predisposed or susceptible to a viral infection. In some embodiments, compounds, compositions comprising a compound, or a combination therapy is administered to a subject that lives in a region where there has been or might be an outbreak with a viral infection. In some embodiments, the viral infection is a latent viral infection. In one embodiment, a compound or a combination therapy is administered to a human infant. In one embodiment, a compound or a combination therapy is administered to a premature human infant. In other embodiments, the viral infection is an active infection. In yet other embodiments, the viral infection is a chronic viral infection. Non-limiting examples of types of virus infections include infections caused by those provided herein.

In a specific embodiment, the viral infection is an enveloped virus infection. In some embodiments, the enveloped virus is a DNA virus. In other embodiments, the enveloped virus is a RNA virus. In some embodiments, the enveloped virus has a double stranded DNA or RNA genome. In other embodiments, the enveloped virus has a single-stranded DNA or RNA genome. In a specific embodiment, the virus infects humans.

In certain embodiments, a compound, a composition comprising a compound, or a combination therapy is administered to a mammal which is 0 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old. In certain embodiments, a compound, a composition comprising a compound, or a combination therapy is administered to a human at risk for a virus infection. In certain embodiments, a compound, a composition comprising a compound, or a combination therapy is administered to a human with a virus infection. In certain embodiments, the patient is a human 0 to 6 months old, 6 to 12 months old, 1 to 5 years old, 5 to 10 years old, 5 to 12 years old, 10 to 15 years old, 15 to 20 years old, 13 to 19 years old, 20 to 25 years old, 25 to 30 years old, 20 to 65 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old. In some embodiments, a compound, a composition comprising a compound, or a combination therapy is administered to a human infant. In other embodiments, a compound, or a combination therapy is administered to a human child. In other embodiments, a compound, a composition comprising a compound, or a combination therapy is administered to a human adult. In yet other embodiments, a compound, a composition comprising a compound, or a combination therapy is administered to an elderly human.

In certain embodiments, a compound, a composition comprising a compound, or a combination therapy is administered to a pet, e.g., a dog or cat. In certain embodiments, a compound, a composition comprising a compound, or a combination therapy is administered to a farm animal or livestock, e.g., pig, cows, horses, chickens, etc. In certain embodiments, a compound, a composition comprising a compound, or a combination therapy is administered to a bird, e.g., ducks or chicken.

In certain embodiments, a compound, a composition comprising a compound, or a combination therapy is administered to a primate, preferably a human, or another mammal, such as a pig, cow, horse, sheep, goat, dog, cat and rodent, in an immunocompromised state or immunosuppressed state or at risk for becoming immunocompromised or immunosuppressed. In certain embodiments, a compound, a composition comprising a compound, or a combination therapy is administered to a subject receiving or recovering from immunosuppressive therapy. In certain embodiments, a compound, a composition comprising a compound, or a combination therapy is administered to a subject that has or is at risk of getting cancer, AIDS, another viral infection, or a bacterial infection. In certain embodiments, a subject that is, will or has undergone surgery, chemotherapy and/or radiation therapy. In certain embodiments, a compound, a composition comprising a compound, or a combination therapy is administered to a subject that has cystic fibrosis, pulmonary fibrosis, or another disease which makes the subject susceptible to a viral infection. In certain embodiments, a compound, a composition comprising a compound, or a combination therapy is administered to a subject that has, will have or had a tissue transplant. In some embodiments, a compound, a composition comprising a compound, or a combination therapy is administered to a subject that lives in a nursing home, a group home (i.e., a home for 10 or more subjects), or a prison. In some embodiments, a compound, a composition comprising a compound, or a combination therapy is administered to a subject that attends school (e.g., elementary school, middle school, junior high school, high school or university) or daycare. In some embodiments, a compound, a composition comprising a compound, or a combination therapy is administered to a subject that works in the healthcare area, such as a doctor or a nurse, or in a hospital. In certain embodiments, a compound, a composition comprising a compound, or a combination therapy is administered to a subject that is pregnant or will become pregnant.

In some embodiments, a patient is administered a compound or a composition comprising a compound, or a combination therapy before any adverse effects or intolerance to therapies other than compounds develops. In some embodiments, compounds or compositions comprising one or more compounds, or combination therapies are administered to refractory patients. In a certain embodiment, refractory patient is a patient refractory to a standard antiviral therapy. In certain embodiments, a patient with a viral infection, is refractory to a therapy when the infection has not significantly been eradicated and/or the symptoms have not been significantly alleviated. The determination of whether a patient is refractory can be made either in vivo or in vitro by any method known in the art for assaying the effectiveness of a treatment of infections, using art-accepted meanings of “refractory” in such a context. In various embodiments, a patient with a viral infection is refractory when viral replication has not decreased or has increased.

In some embodiments, compounds or compositions comprising one or more compounds, or combination therapies are administered to a patient to prevent the onset or reoccurrence of viral infections in a patient at risk of developing such infections. In some embodiments, compounds or compositions comprising one or more compounds, or combination therapies are administered to a patient who is susceptible to adverse reactions to conventional therapies.

In some embodiments, one or more compounds or compositions comprising one or more compounds, or combination therapies are administered to a patient who has proven refractory to therapies other than compounds, but are no longer on these therapies. In certain embodiments, the patients being managed or treated in accordance with the methods of this invention are patients already being treated with antibiotics, anti-virals, anti-fungals, or other biological therapy/immunotherapy. Among these patients are refractory patients, patients who are too young for conventional therapies, and patients with reoccurring viral infections despite management or treatment with existing therapies.

In some embodiments, the subject being administered one or more compounds or compositions comprising one or more compounds, or combination therapies has not received a therapy prior to the administration of the compounds or compositions or combination therapies. In other embodiments, one or more compounds or compositions comprising one or more compounds, or combination therapies are administered to a subject who has received a therapy prior to administration of one or more compounds or compositions comprising one or more compounds, or combination therapies. In some embodiments, the subject administered a compound or a composition comprising a compound was refractory to a prior therapy or experienced adverse side effects to the prior therapy or the prior therapy was discontinued due to unacceptable levels of toxicity to the subject.

7.2 Mode of Administration

When administered to a patient, a compound is preferably administered as a component of a composition that optionally comprises a pharmaceutically acceptable vehicle. The composition can be administered orally, or by any other convenient route, for example, by infusion or bolus injection, by absorption through epithelial or mucocutaneous linings (e.g., oral mucosa, rectal, and intestinal mucosa) and may be administered together with another biologically active agent. Administration can be systemic or local. Various delivery systems are known, e.g., encapsulation in liposomes, microparticles, microcapsules, capsules, and can be used to administer the compound and pharmaceutically acceptable salts thereof.

Methods of administration include but are not limited to parenteral, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The mode of administration is left to the discretion of the practitioner. In most instances, administration will result in the release of a compound into the bloodstream.

In specific embodiments, it may be desirable to administer a compound locally. This may be achieved, for example, and not by way of limitation, by local infusion, topical application, e.g., in conjunction with a wound dressing, by injection, by means of a catheter, by means of a suppository, or by means of an implant, said implant being of a porous, non-porous, or gelatinous material, including membranes, such as sialastic membranes, or fibers. In such instances, administration may selectively target a local tissue without substantial release of a compound into the bloodstream.

In certain embodiments, it may be desirable to introduce a compound into the central nervous system by any suitable route, including intraventricular, intrathecal and epidural injection. Intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, a compound is formulated as a suppository, with traditional binders and vehicles such as triglycerides.

For viral infections with cutaneous manifestations, the compound can be administered topically. Similarly, for viral infections with ocular manifestation, the compounds can be administered ocularly.

In another embodiment, a compound is delivered in a vesicle, in particular a liposome (See Langer, 1990, Science 249:1527 1533; Treat et al., in Liposomes in the Therapy of Infectious Disease and Bacterial infection, Lopez-Berestein and Fidler (eds.), Liss, New York, pp. 353 365 (1989); Lopez Berestein, ibid., pp. 317 327; See generally ibid.).

In another embodiment, a compound is delivered in a controlled release system (See, e.g., Goodson, in Medical Applications of Controlled Release, supra, vol. 2, pp. 115 138 (1984)). Examples of controlled-release systems are discussed in the review by Langer, 1990, Science 249:1527 1533 may be used. In one embodiment, a pump may be used (See Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (See Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; See also Levy et al., 1985, Science 228:190; During et al., 1989, Ann. Neurol. 25:351; Howard et al., 1989, J. Neurosurg. 71:105). In a specific embodiment, a controlled-release system comprising a compound is placed in close proximity to the tissue infected with a virus to be prevented, treated and/or managed. In accordance with this embodiment, the close proximity of the controlled-release system to the infection may result in only a fraction of the dose of the compound required if it is systemically administered.

In certain embodiments, it may be preferable to administer a compound via the natural route of infection of the virus against which a compound has antiviral activity. For example, it may be desirable to administer a compound of the invention into the lungs by any suitable route to treat or prevent an infection of the respiratory tract by viruses (e.g., influenza virus). Pulmonary administration can also be employed, e.g., by use of an inhaler or nebulizer, and formulation with an aerosolizing agent for use as a spray.

7.3 Agents for Use in Combination with Compounds

Therapeutic or prophylactic agents that can be used in combination with compounds for the prevention, treatment and/or management of a viral infection include, but are not limited to, small molecules, synthetic drugs, peptides (including cyclic peptides), polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides), antibodies, synthetic or natural inorganic molecules, mimetic agents, and synthetic or natural organic molecules. Specific examples of such agents include, but are not limited to, immunomodulatory agents (e.g., interferon), anti-inflammatory agents (e.g., adrenocorticoids, corticosteroids (e.g., beclomethasone, budesonide, flunisolide, fluticasone, triamcinolone, methylprednisolone, prednisolone, prednisone, hydrocortisone), glucocorticoids, steriods, and non-steriodal anti-inflammatory drugs (e.g., aspirin, ibuprofen, diclofenac, and COX-2 inhibitors), pain relievers, leukotreine antagonists (e.g., montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), anti-viral agents (e.g., nucleoside analogs (e.g., zidovudine, acyclovir, gangcyclovir, vidarabine, idoxuridine, trifluridine, and ribavirin), foscarnet, amantadine, rimantadine, saquinavir, indinavir, ritonavir, and AZT) and antibiotics (e.g., dactinomycin (formerly actinomycin), bleomycin, erythomycin, penicillin, mithramycin, and anthramycin (AMC)).

Any therapy which is known to be useful, or which has been used or is currently being used for the prevention, management, and/or treatment of a viral infection or can be used in combination with compounds in accordance with the invention described herein. See, e.g., Gilman et al., Goodman and Gilman's: The Pharmacological Basis of Therapeutics, 10th ed., McGraw-Hill, New York, 2001; The Merck Manual of Diagnosis and Therapy, Berkow, M. D. et al. (eds.), 17th Ed., Merck Sharp & Dohme Research Laboratories, Rahway, N.J., 1999; Cecil Textbook of Medicine, 20th Ed., Bennett and Plum (eds.), W.B. Saunders, Philadelphia, 1996, and Physicians' Desk Reference (61^{st} ed. 1007) for information regarding therapies (e.g., prophylactic or therapeutic agents) which have been or are currently being used for preventing, treating and/or managing viral infections.

7.3.1 Antiviral Agents

Antiviral agents that can be used in combination with compounds include, but are not limited to, non-nucleoside reverse transcriptase inhibitors, nucleoside reverse transcriptase inhibitors, protease inhibitors, and fusion inhibitors. In one embodiment, the antiviral agent is selected from the group consisting of amantadine, oseltamivir phosphate, rimantadine, and zanamivir. In another embodiment, the antiviral agent is a non-nucleoside reverse transcriptase inhibitor selected from the group consisting of delavirdine, efavirenz, and nevirapine. In another embodiment, the antiviral agent is a nucleoside reverse transcriptase inhibitor selected from the group consisting of abacavir, didanosine, emtricitabine, emtricitabine, lamivudine, stavudine, tenofovir DF, zalcitabine, and zidovudine. In another embodiment, the antiviral agent is a protease inhibitor selected from the group consisting of amprenavir, atazanavir, fosamprenav, indinavir, lopinavir, nelfinavir, ritonavir, and saquinavir. In another embodiment, the antiviral agent is a fusion inhibitor such as enfuvirtide.

Additional, non-limiting examples of antiviral agents for use in combination compounds include the following: rifampicin, nucleoside reverse transcriptase inhibitors (e.g., AZT, ddI, ddC, 3TC, d4T), non-nucleoside reverse transcriptase inhibitors (e.g., delavirdine efavirenz, nevirapine), protease inhibitors (e.g., aprenavir, indinavir, ritonavir, and saquinavir), idoxuridine, cidofovir, acyclovir, ganciclovir, zanamivir, amantadine, and palivizumab. Other examples of anti-viral agents include but are not limited to acemannan; acyclovir; acyclovir sodium; adefovir; alovudine; alvircept sudotox; amantadine hydrochloride (SYMMETREL™); aranotin; arildone; atevirdine mesylate; pyridine; cidofovir; cipamfylline; cytarabine hydrochloride; delavirdine mesylate; desciclovir; didanosine; disoxaril; edoxudine; enviradene; enviroxime; famciclovir; famotine hydrochloride; fiacitabine; fialuridine; fosarilate; foscamet sodium; fosfonet sodium; ganciclovir; ganciclovir sodium; idoxuridine; kethoxal; lamivudine; lobucavir; memotine hydrochloride; methisazone; nevirapine; oseltamivir phosphate (TAMIFLU™); penciclovir; pirodavir; ribavirin; rimantadine hydrochloride (FLUMADINE™); saquinavir mesylate; somantadine hydrochloride; sorivudine; statolon; stavudine; tilorone hydrochloride; trifluridine; valacyclovir hydrochloride; vidarabine; vidarabine phosphate; vidarabine sodium phosphate; viroxime; zalcitabine; zanamivir (RELENZA™); zidovudine; and zinviroxime.

7.3.2 Antibacterial Agents

Antibacterial agents, including antibiotics, that can be used in combination with compounds include, but are not limited to, aminoglycoside antibiotics, glycopeptides, amphenicol antibiotics, ansamycin antibiotics, cephalosporins, cephamycins oxazolidinones, penicillins, quinolones, streptogamins, tetracyclins, and analogs thereof. In some embodiments, antibiotics are administered in combination with a compound to prevent and/or treat a bacterial infection.

In a specific embodiment, compounds are used in combination with other protein synthesis inhibitors, including but not limited to, streptomycin, neomycin, erythromycin, carbomycin, and spiramycin.

In one embodiment, the antibacterial agent is selected from the group consisting of ampicillin, amoxicillin, ciprofloxacin, gentamycin, kanamycin, neomycin, penicillin G, streptomycin, sulfanilamide, and vancomycin. In another embodiment, the antibacterial agent is selected from the group consisting of azithromycin, cefonicid, cefotetan, cephalothin, cephamycin, chlortetracycline, clarithromycin, clindamycin, cycloserine, dalfopristin, doxycycline, erythromycin, linezolid, mupirocin, oxytetracycline, quinupristin, rifampin, spectinomycin, and trimethoprim.

Additional, non-limiting examples of antibacterial agents for use in combination with compounds include the following: aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefinetazole, and cefminox), folic acid analogs (e.g., trimethoprim), glycopeptides (e.g., vancomycin), lincosamides (e.g., clindamycin, and lincomycin), macrolides (e.g., azithromycin, carbomycin, clarithomycin, dirithromycin, erythromycin, and erythromycin acistrate), monobactams (e.g., aztreonam, carumonam, and tigemonam), nitrofurans (e.g., furaltadone, and furazolium chloride), oxacephems (e.g., flomoxef, and moxalactam), oxazolidinones (e.g., linezolid), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o benethamine, penicillin 0, penicillin V, penicillin V benzathine, penicillin V hydrabamine, penimepicycline, and phencihicillin potassium), quinolones and analogs thereof (e.g., cinoxacin, ciprofloxacin, clinafloxacin, flumequine, grepagloxacin, levofloxacin, and moxifloxacin), streptogramins (e.g., quinupristin and dalfopristin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), and tetracyclines (e.g., apicycline, chlortetracycline, clomocycline, and demeclocycline). Additional examples include cycloserine, mupirocin, tuberin amphomycin, bacitracin, capreomycin, colistin, enduracidin, enviomycin, and 2,4 diaminopyrimidines (e.g., brodimoprim).

7.4 Dosages & Frequency of Administration

The amount of a compound, or the amount of a composition comprising a compound, that will be effective in the prevention, treatment and/or management of a viral infection can be determined by standard clinical techniques. In vitro or in vivo assays may optionally be employed to help identify optimal dosage ranges. The precise dose to be employed will also depend, e.g., on the route of administration, the type of invention, and the seriousness of the infection, and should be decided according to the judgment of the practitioner and each patient's or subject's circumstances.

In some embodiments, the dosage of a compound is determined by extrapolating from the no observed adverse effective level (NOAEL), as determined in animal studies. This extrapolated dosage is useful in determining the maximum recommended starting dose for human clinical trials. For instance, the NOAELs can be extrapolated to determine human equivalent dosages (HED). Typically, HED is extrapolated from a non-human animal dosage based on the doses that are normalized to body surface area (i.e., mg/m²). In specific embodiments, the NOAELs are determined in mice, hamsters, rats, ferrets, guinea pigs, rabbits, dogs, primates, primates (monkeys, marmosets, squirrel monkeys, baboons), micropigs or minipigs. For a discussion on the use of NOAELs and their extrapolation to determine human equivalent doses, See Guidance for Industry Estimating the Maximum Safe Starting Dose in Initial Clinical Trials for Therapeutics in Adult Healthy Volunteers, U.S. Department of Health and Human Services Food and Drug Administration Center for Drug Evaluation and Research (CDER), Pharmacology and Toxicology, July 2005. In one embodiment, a compound or composition thereof is administered at a dose that is lower than the human equivalent dosage (HED) of the NOAEL over a period of 1 week, 2 weeks, 3 weeks, 1 month, 2 months, three months, four months, six months, nine months, 1 year, 2 years, 3 years, 4 years or more.

In certain embodiments, a dosage regime for a human subject can be extrapolated from animal model studies using the dose at which 10% of the animals die (LD10). In general the starting dose of a Phase I clinical trial is based on preclinical testing. A standard measure of toxicity of a drug in preclinical testing is the percentage of animals that die because of treatment. It is well within the skill of the art to correlate the LD10 in an animal study with the maximal-tolerated dose (MTD) in humans, adjusted for body surface area, as a basis to extrapolate a starting human dose. In some embodiments, the interrelationship of dosages for one animal model can be converted for use in another animal, including humans, using conversion factors (based on milligrams per meter squared of body surface) as described, e.g., in Freireich et al., Cancer Chemother. Rep., 1966, 50:219-244. Body surface area may be approximately determined from height and weight of the patient. See, e.g., Scientific Tables, Geigy Pharmaceuticals, Ardley, N.Y., 1970, 537. In certain embodiments, the adjustment for body surface area includes host factors such as, for example, surface area, weight, metabolism, tissue distribution, absorption rate, and excretion rate. In addition, the route of administration, excipient usage, and the specific disease or virus to target are also factors to consider. In one embodiment, the standard conservative starting dose is about 1/10 the murine LD10, although it may be even lower if other species (i.e., dogs) were more sensitive to the compound. In other embodiments, the standard conservative starting dose is about 1/100, 1/95, 1/90, 1/85, 1/80, 1/75, 1/70, 1/65, 1/60, 1/55, 1/50, 1/45, 1/40, 1/35, 1/30, 1/25, 1/20, 1/15, 2/10, 3/10, 4/10, or 5/10 of the murine LD10. In other embodiments, a starting dose amount of a compound in a human is lower than the dose extrapolated from animal model studies. In another embodiment, an starting dose amount of a compound in a human is higher than the dose extrapolated from animal model studies. It is well within the skill of the art to start doses of the active composition at relatively low levels, and increase or decrease the dosage as necessary to achieve the desired effect with minimal toxicity.

Exemplary doses of compounds or compositions include milligram or microgram amounts per kilogram of subject or sample weight (e.g., about 1 microgram per kilogram to about 500 milligrams per kilogram, about 5 micrograms per kilogram to about 100 milligrams per kilogram, or about 1 microgram per kilogram to about 50 micrograms per kilogram). In specific embodiments, a daily dose is at least 50 mg, 75 mg, 100 mg, 150 mg, 250 mg, 500 mg, 750 mg, or at least 1 g.

In one embodiment, the dosage is a concentration of 0.01 to 5000 mM, 1 to 300 mM, 10 to 100 mM and 10 mM to 1 M. In another embodiment, the dosage is a concentration of at least 5 μM, at least 10 μM, at least 50 μM, at least 100 μM, at least 500 μM, at least 1 mM, at least 5 mM, at least 10 mM, at least 50 mM, at least 100 mM, or at least 500 mM.

In one embodiment, the dosage is a concentration of 0.01 to 5000 mM, 1 to 300 mM, 10 to 100 mM and 10 mM to 1 M. In another embodiment, the dosage is a concentration of at least 5 μM, at least 10 μM, at least 50 μM, at least 100 μM, at least 500 μM, at least 1 mM, at least 5 mM, at least 10 mM, at least 50 mM, at least 100 mM, or at least 500 mM. In a specific embodiment, the dosage is 0.25 μg/kg or more, preferably 0.5 μg/kg or more, 1 μg/kg or more, 2 μg/kg or more, 3 μg/kg or more, 4 μg/kg or more, 5 μg/kg or more, 6 μg/kg or more, 7 μg/kg or more, 8 μg/kg or more, 9 μg/kg or more, or 10 μg/kg or more, 25 μg/kg or more, preferably 50 μg/kg or more, 100 μg/kg or more, 250 μg/kg or more, 500 μg/kg or more, 1 mg/kg or more, 5 mg/kg or more, 6 mg/kg or more, 7 mg/kg or more, 8 mg/kg or more, 9 mg/kg or more, or 10 mg/kg or more of a patient's body weight.

In another embodiment, the dosage is a unit dose of 5 mg, preferably 10 mg, 50 mg, 100 mg, 150 mg, 200 mg, 250 mg, 300 mg, 350 mg, 400 mg, 500 mg, 550 mg, 600 mg, 650 mg, 700 mg, 750 mg, 800 mg or more. In another embodiment, the dosage is a unit dose that ranges from about 5 mg to about 100 mg, about 100 mg to about 200 μg, about 150 mg to about 300 mg, about 150 mg to about 400 mg, 250 μg to about 500 mg, about 500 mg to about 800 mg, about 500 mg to about 1000 mg, or about 5 mg to about 1000 mg.

In certain embodiments, suitable dosage ranges for oral administration are about 0.001 milligram to about 500 milligrams of a compound, per kilogram body weight per day. In specific embodiments of the invention, the oral dose is about 0.01 milligram to about 100 milligrams per kilogram body weight per day, about 0.1 milligram to about 75 milligrams per kilogram body weight per day or about 0.5 milligram to 5 milligrams per kilogram body weight per day. The dosage amounts described herein refer to total amounts administered; that is, if more than one compound is administered, then, in some embodiments, the dosages correspond to the total amount administered. In a specific embodiment, oral compositions contain about 10% to about 95% a compound of the invention by weight.

Suitable dosage ranges for intravenous (i.v.) administration are about 0.01 milligram to about 100 milligrams per kilogram body weight per day, about 0.1 milligram to about 35 milligrams per kilogram body weight per day, and about 1 milligram to about 10 milligrams per kilogram body weight per day. In some embodiments, suitable dosage ranges for intranasal administration are about 0.01 pg/kg body weight per day to about 1 mg/kg body weight per day. Suppositories generally contain about 0.01 milligram to about 50 milligrams of a compound of the invention per kilogram body weight per day and comprise active ingredient in the range of about 0.5% to about 10% by weight.

Recommended dosages for intradermal, intramuscular, intraperitoneal, subcutaneous, epidural, sublingual, intracerebral, intravaginal, transdermal administration or administration by inhalation are in the range of about 0.001 milligram to about 500 milligrams per kilogram of body weight per day. Suitable doses for topical administration include doses that are in the range of about 0.001 milligram to about 50 milligrams, depending on the area of administration. Effective doses may be extrapolated from dose-response curves derived from in vitro or animal model test systems. Such animal models and systems are well known in the art.

In another embodiment, a subject is administered one or more doses of a prophylactically or therapeutically effective amount of a compound or a composition, wherein the prophylactically or therapeutically effective amount is not the same for each dose. In another embodiment, a subject is administered one or more doses of a prophylactically or therapeutically effective amount of a compound or a composition,
wherein the dose of a prophylactically or therapeutically effective amount administered to said subject is increased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, or 50 μg/kg, as treatment progresses. In another embodiment, a subject is administered one or more doses of a prophylactically or therapeutically effective amount of a compound or composition, wherein the dose is decreased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, or 50 μg/kg, as treatment progresses.

In certain embodiments, a subject is administered a compound or a composition in an amount effective to inhibit or reduce viral genome replication by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In other embodiments, a subject is administered a compound or a composition in an amount effective to inhibit or reduce viral genome replication by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In certain embodiments, a subject is administered a compound or a composition in an amount effective to inhibit or reduce viral genome replication by at least 1.5 fold, 2 fold, 2.5 fold, 3 fold, 4 fold, 5 fold, 8 fold, 10 fold, 15 fold, 20 fold, or 2 to 5 fold, 2 to 10 fold, 5 to 10 fold, or 5 to 20 fold relative to a negative control as determined using an assay described herein or other known to one of skill in the art.

In certain embodiments, a subject is administered a compound or a composition in an amount effective to inhibit or reduce viral protein synthesis by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In other embodiments, a subject is administered a compound or a composition in an amount effective to inhibit or reduce viral protein synthesis by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In certain embodiments, a subject is administered a compound or a composition in an amount effective to inhibit or reduce viral protein synthesis by at least 1.5 fold, 2 fold, 2.5 fold, 3 fold, 4 fold, 5 fold, 8 fold, 10 fold, 15 fold, 20 fold, or 2 to 5 fold, 2 to 10 fold, 5 to 10 fold, or 5 to 20 fold relative to a negative control as determined using an assay described herein or others known to one of skill in the art.

In certain embodiments, a subject is administered a compound or a composition in an amount effective to inhibit or reduce viral infection by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In some embodiments, a subject is administered a compound or a composition in an amount effective to inhibit or reduce viral infection by at least 1.5 fold, 2 fold, 2.5 fold, 3 fold, 4 fold, 5 fold, 8 fold, 10 fold, 15 fold, 20 fold, or 2 to 5 fold, 2 to 10 fold, 5 to 10 fold, or 5 to 20 fold relative to a negative control as determined using an assay described herein or others known to one of skill in the art.

In certain embodiments, a subject is administered a compound or a composition in an amount effective to inhibit or reduce viral replication by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In some embodiments, a subject is administered a compound or a composition in an amount effective to inhibit or reduce viral replication by at least 1.5 fold, 2 fold, 2.5 fold, 3 fold, 4 fold, 5 fold, 8 fold, 10 fold, 15 fold, 20 fold, or 2 to 5 fold, 2 to 10 fold, 5 to 10 fold, or 5 to 20 fold relative to a negative control as determined using an assay described herein or others known to one of skill in the art. In other embodiments, a subject is administered a compound or a composition in an amount effective to inhibit or reduce viral replication by 1 log, 1.5 logs, 2 logs, 2.5 logs, 3 logs, 3.5 logs, 4 logs, 5 logs or more relative to a negative control as determined using an assay described herein or others known to one of skill in the art.

In certain embodiments, a subject is administered a compound or a composition in an amount effective to inhibit or reduce the ability of the virus to spread to other individuals by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In other embodiments, a subject is administered a compound or a composition in an amount effective to inhibit or reduce the ability of the virus to spread to other cells, tissues or organs in the subject by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art.

In certain embodiments, a dose of a compound or a composition is administered to a subject every day, every other day, every couple of days, every third day, once a week, twice a week, three times a week, or once every two weeks. In other embodiments, two, three or four doses of a compound or a composition is administered to a subject every day, every couple of days, every third day, once a week or once every two weeks. In some embodiments, a dose(s) of a compound or a composition is administered for 2 days, 3 days, 5 days, 7 days, 14 days, or 21 days. In certain embodiments, a dose of a compound or a composition is administered for 1 month, 1.5 months, 2 months, 2.5 months, 3 months, 4 months, 5 months, 6 months or more.

The dosages of prophylactic or therapeutic agents which have been or are currently used for the prevention, treatment and/or management of a viral infection can be determined using references available to a clinician such as, e.g., the Physicians' Desk Reference (61^{st} ed. 2007). Preferably, dosages lower than those which have been or are currently being used to prevent, treat and/or manage the infection are utilized in combination with one or more compounds or compositions.

For compounds which have been approved for uses other than prevention, treatment or management of viral infections, safe ranges of doses can be readily determined using references available to clinicians, such as e.g., the Physician's Desk Reference (61^{st} ed. 2007).

The above-described administration schedules are provided for illustrative purposes only and should not be considered limiting. A person of ordinary skill in the art will readily understand that all doses are within the scope of the invention.

It is to be understood and expected that variations in the principles of invention herein disclosed may be made by one skilled in the art and it is intended that such modifications are to be included within the scope of the present invention.

Throughout this application, various publications are referenced in parentheses. The disclosures of these publications in their entireties are hereby incorporated by reference into this application to more fully describe the state of the art to which this invention pertains. The following examples further illustrate the invention, but should not be construed to limit the scope of the invention in any way.

### EXAMPLES

### Example 1

### Torin1 Inhibits the Production of HCMV Progeny

To determine the effects of the mTOR inhibitor, Torin1, on HCMV replication, fibroblasts were growth arrested by serum starvation, infected with HCMV, and treated with either Torin1 or rapamycin, and growth was monitored over multiple rounds of viral replication.

Primary human foreskin fibroblasts were grown in Dulbecco's modified Eagle's medium (DMEM) containing 10% normal calf serum and used between passages 6 and 14. Multistep growth analysis of viruses was performed by plating human fibroblasts at confluence and serum starved for 48 h prior to infection. Cells were infected at a multiplicity of 0.05 PFU/cell with HCMV. Cells in six-well plates were incubated with virus in 300 μl of medium for 1 h with rocking every 15 min. After adsorption, the inoculum was removed and replaced with fresh serum-free medium. The amount of virus present in cell-free supernatants was quantified by the 50% tissue culture infective dose (TCID₅₀) method on primary human fibroblasts.

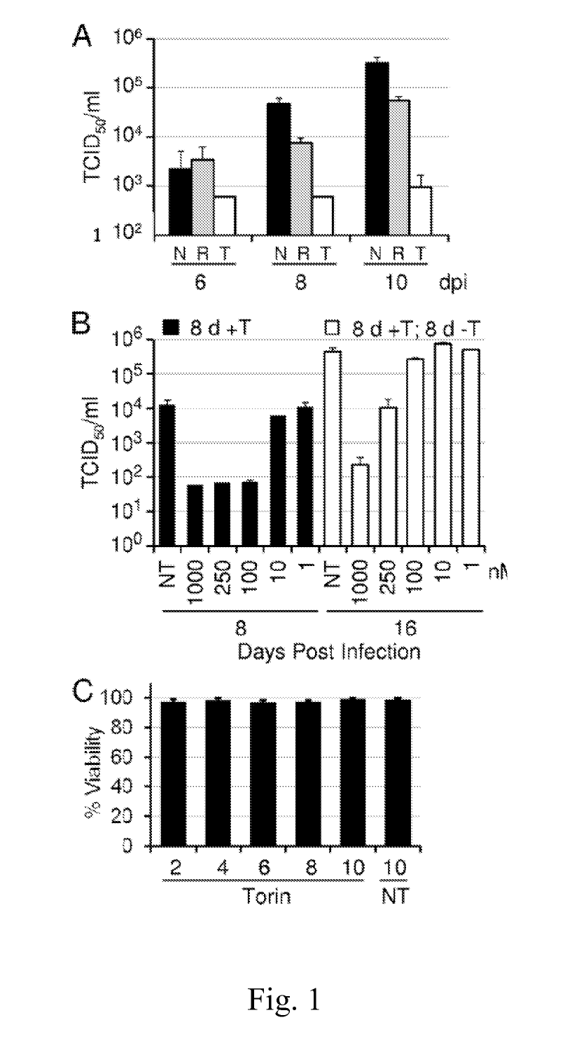

As shown in FIG. 1A, rapamycin treatment modestly inhibited HCMV replication, achieving about an 8-fold effect on day 10. In contrast, Torin1 reduced the yield of HCMV by a factor of about 160 on day 10. Torin1 was effective in blocking the production of HCMV progeny over a range of concentrations, with a 50% inhibitory concentration (IC50) of about 60 nM (8 days post infection) (FIG. 1B). This dose compares favorably with the IC50s of 2 to 10 nM at which Torin1 inhibits the kinase activities of mTORC1 and mTORC2 (47).

Previous reports have shown that although Torin1 substantially blocks cellular proliferation, it does not kill cells at concentrations of up to 500 nM. We tested the effect of 250 nM Torin1 on the viability of growth-arrested fibroblasts using a trypan blue exclusion assay. Torin1 treatment did not affect the viability of these cells, with more than 95% of the cells remaining viable over 10 days of Torin1 treatment (FIG. 1C).

To further confirm that the viral growth defect was not the result of cytotoxicity, we performed a drug release experiment. Infected cells were treated with a range of concentrations of Torin1 for 8 days, after which the cells were maintained in medium lacking Torin1. Eight days later, virus in the supernatant was quantified by the TCID50 method (16 days post infection) (FIG. 1B). Following the removal of the drug, HCMV replication partially recovered in cultures that had initially received 1 mM drug, substantially recovered in cells that had received 250 nM drug, and completely recovered in cells that had received 100 nM Torin1. The >100-fold increase in virus yield after the reversal of an 8-day Torin1 treatment further demonstrates that cells treated with <250 nM drug remained viable.

These results demonstrate that Torin1 is a potent inhibitor of HCMV replication. Given data from previous work demonstrating the selectivity of Torin1 for the mTOR kinase and its ability to inhibit rapamycin-resistant mTORC1 activity it is likely that this mTORC1 activity is important for HCMV lytic replication.

### Example 2

### Torin1 Blocks the Accumulation of Viral DNA and a Late Viral Protein

To determine the nature of the blockade in the viral life cycle imposed by Torin1, we initially examined the impact of drug treatment at a dose of 250 nM on HCMV entry. Cells were either pretreated for 24 h with Torin1 or treated with drug immediately following viral adsorption.

Determination of viral DNA and transcript accumulation in infected cells. The accumulation of viral DNA during HCMV infection was monitored by quantitative PCR (qPCR) as described previously (Terhune, et al. (2007) J. Virol. 81:3109-3123).

Briefly, primary human fibroblasts were infected with BADinGFP at a multiplicity of 0.05 PFU/cell. At the indicated times, cells were harvested by scraping them into medium and were stored as frozen cell pellets until analysis. Cell pellets were resuspended in 500 μl of a solution containing 400 mM NaCl, 10 mM Tris (pH 7.5), and 10 mM EDTA. Proteinase K (20 μg) was added together with 4 μl of a 20% SDS solution. The lysate was incubated overnight at 37° C. Lysates were phenolchloroform extracted. RNase A was added (20 μg), and the lysates were incubated at 37° C. for 1 h. Lysates were extracted with phenol-chloroform and then with chloroform. DNA was precipitated by the addition of 1 ml of 100% ethanol followed by centrifugation at 14,000×g for 30 min. DNA was washed once in 70% ethanol prior to resuspension in 501 of 10 mM Tris (pH 7.5). For each sample, DNA was quantified by using a NanoDrop spectrophotometer (Thermo Scientific). Five hundred nanograms of DNA was added to 12.5 μl 2×SYBR green PCR master mix (Applied Biosystems) and 2 M each primer in a total volume of 25 μl. As an additional control for equal loading, the amount of viral DNA in each sample was normalized to the amount of the cellular glyceraldehyde-3-phosphate dehydrogenase (GAPDH) gene in each sample.

Western blot analysis of proteins was performed on human fibroblast pretreated for 24 h with Torin1 or treated with drug for 1 h following viral adsorption. Cells were lysed in radioimmunoprecipitation assay (RIPA) buffer (50 mM Tris-HCl [pH 7.4], 1% NP-40, 0.25% sodium deoxycholate, 150 mM NaCl, 1 mM EDTA) containing protease inhibitors (complete EDTA free; Roche). Protein concentrations in each lysate were determined by the Bradford assay. 30 g of protein was analyzed per sample. Proteins in cell lysates were resolved on SDS-containing 10% polyacrylamide gels. Proteins were transferred onto Protran membranes by using a semidry transfer apparatus. Membranes were blocked with phosphate-buffered saline containing 0.1% Tween 20 (PBS-T) and 5% fat-free dried milk for 1 h prior to incubation with primary antibody. Anti-IE1 monoclonal antibodies (56) or anti-tubulin antibodies (Sigma) were diluted in PBS-T containing 1% bovine serum albumin (BSA) and incubated with the membrane for 1 h at room temperature. Following extensive washing with PBS-T, blots were incubated with goat anti-mouse horseradish peroxidase (HRP)-coupled secondary antibodies diluted 1:5,000 in PBS-T containing 1% BSA. Membranes were then washed again in PBS-T, and proteins were visualized by chemiluminescence using ECL reagent (Amersham).

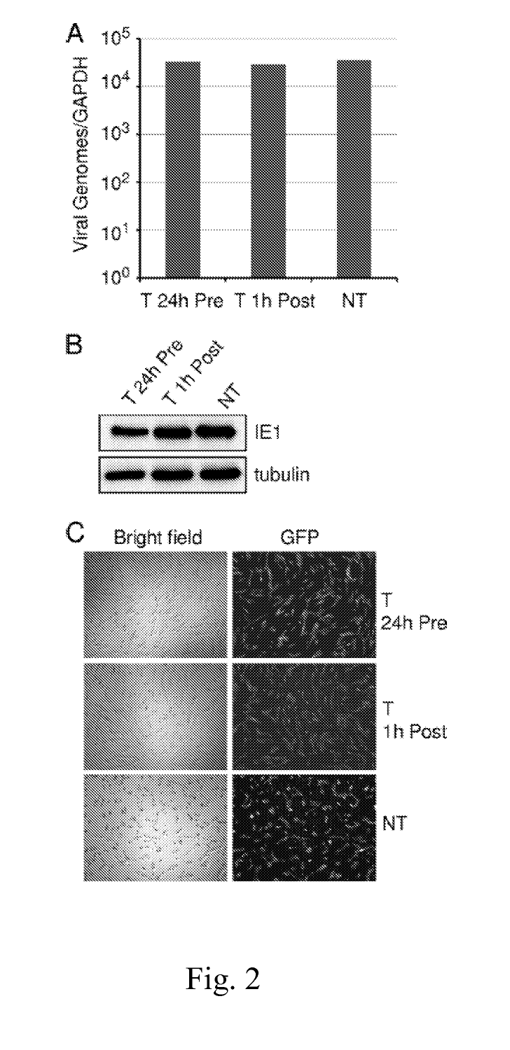

The level of cell-associated viral DNA at 2 h post infection (hpi) was not influenced by either drug treatment regimen (FIG. 2A). When the expression of the HCMV immediate-early IE1 protein was examined under these conditions, there was no appreciable difference in the amount of IE1 relative to cell-coded tubulin between the Torin1-treated and untreated cells at 6 hpi (FIG. 2B). Furthermore, drug treatment did not alter the percentage of cells expressing a GFP marker protein expressed from the virus genome at 24 hpi (FIG. 2C). Together, these results demonstrate that the initial steps of the HCMV life cycle, including the binding and entry of the virion and the expression of an immediate-early protein, are not affected by Torin1.

### Example 3

### The Effect of Torin1 Compared to Rapamycin on the Accumulation of Viral Proteins

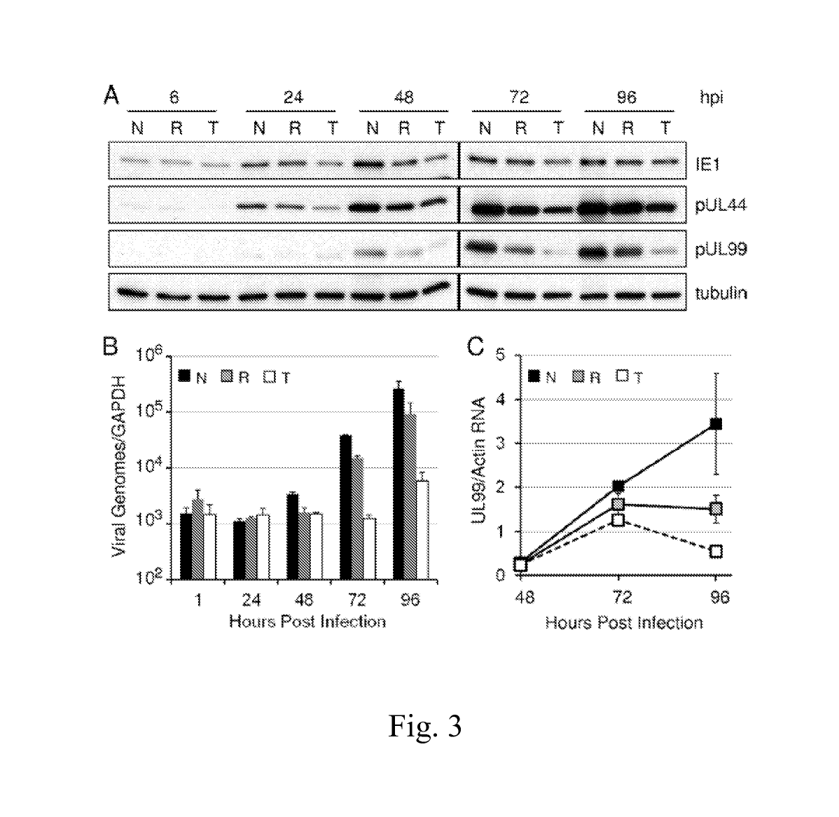

The effect of Torin1 compared to rapamycin on the accumulation of representative viral proteins from each kinetic class (IE1, pUL44, and pUL99) at 6 to 96 hpi was examined by western blot (FIG. 3A) as described above. Antibodies to pUL99 have been described previously (Silva, et al. J. Virol. (2003) 77:10594-10605). In addition, accumulation of HCMV DNA was monitored following infection using the methods described above.

The UL99 transcript levels following infection were determined as described previously (Depto, et al. (1992) J. Virol. 66:3241-3246). Briefly, total RNA was harvested at the indicated times by Trizol (Invitrogen) extraction. DNase-treated RNA (0.5 μg) was reverse transcribed with the TaqMan reverse transcription reagent kit (Applied Biosciences) using random hexamer primers. Two microliters of cDNA was added to SYBR green master mix (Applied Biosciences) together with primers specific for UL99 (5′-GTGTCCCATTCCCGACTCG-3′ (SEQ ID NO:4) and 5′-TTCACAACGTCCACCCACC-3′ (SEQ ID NO:5). Actin levels were measured in the same samples by using the following primers: 5′-TCCTCCTGAGCGCAAGTACTC-3′ (SEQ ID NO:6) and 5′-CGGACTCGTCATACTCCT GCTT-3′ (SEQ ID NO:7). Copy numbers for UL99 and actin transcripts were determined by comparing the threshold cycle for each sample to a standard curve, which consisted of serial dilutions of a recombinant HCMV BAC that contains the actin gene inserted into the UL21.5 locus. The standard curve for all experiments had an R value greater than 0.98.

Rapamycin had little effect on the accumulation of the immediate-early protein IE1 and the early protein pUL44, and it reduced the level of the late protein pUL99 to a modest extent (FIG. 3A). Torin1 inhibited the accumulation of IE1 and pUL44 to a limited extent, but it dramatically reduced the amount of pUL99. Since the expression of pUL99 is dependent on the initiation of viral DNA replication, we tested whether Torin1 inhibits viral DNA accumulation (FIG. 3B). Viral DNA accumulation was measured by quantitative real-time PCR of fibroblasts treated with rapamycin or Torin1. Rapamycin modestly inhibited viral DNA accumulation, consistent with its effect on the production of HCMV progeny. In contrast, Torin1 reduced viral DNA accumulation at 96 hpi by 150-fold. This finding suggested that the inhibition of viral late protein expression reflects a reduced transcription of viral late RNAs due to the inhibition of viral DNA accumulation. To test this hypothesis, we measured the levels of expression of UL99 mRNA in the presence of Torin1 and rapamycin. Both rapamycin and Torin1 decreased the levels of UL99 mRNA, and Torin1 had a greater effect than rapamycin (FIG. 3C). The decreased level of UL99 mRNA in Torin1-treated cells is consistent with the observed inhibition of viral DNA accumulation. The decrease in UL99 protein levels may be more severe than the decrease in UL99 mRNA levels, raising the possibility that mTOR activity might play a role in viral late protein synthesis specifically. However, an interpretation of these results in terms of an effect on late translation is confounded by the drug's effect on DNA accumulation. In sum, these results demonstrate that a rapamycin-insensitive mTOR activity is required for efficient HCMV DNA accumulation but is dispensable for the expression of viral immediate-early and early proteins.

### Example 4

### Torin1 Blocks the Phosphorylation of 4EBP1 within HCMV-Infected Cells

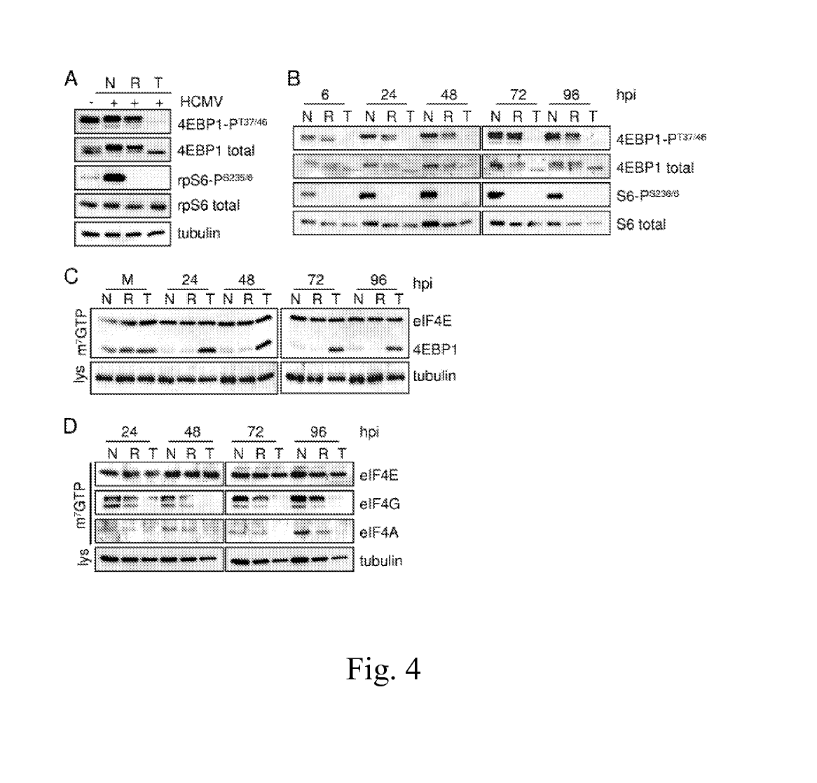

The effect of Torin1 on the phosphorylation of mTORC1 targets during HCMV infection was investigated. HCMV infection induces mTORC1 activity, but the phosphorylation of mTORC1 targets is differentially sensitive to the mTORC1 inhibitor rapamycin. While the mTORC1 phosphorylation of p70 S6 kinase, and its subsequent phosphorylation of rpS6, is inhibited by rapamycin during HCMV infection, the phosphorylation of another mTORC1 target, 4EBP1, is resistant to rapamycin. This differential effect on mTOR targets could indicate that a kinase other than mTOR is responsible for 4EBP1 phosphorylation during infection. To test this possibility, fibroblasts infected with rapamycin were treated with Torin1 and the phosphorylation status of 4EBP1 and rpS6 was measured. Both drugs markedly inhibited the induction of rpS6 phosphorylation that is normally observed during HCMV infection, but only Torin1 substantially blocked the phosphorylation of 4EBP1 (FIG. 4A). This was evident both by the failure to detect phosphorylated 4EBP1-PT37/46 by using an antibody specific for the phosphoform and by the altered migration of total 4EBP1 in the presence of the drug. Total 4EBP1, rpS6, and tubulin levels were monitored to control for protein recovery. The differential effects of the drugs were observed throughout the course of infection (FIG. 4B). These results demonstrate that the rapamycin-resistant phosphorylation of 4EBP1 during HCMV infection is dependent on Torin1-sensitive mTOR activity rather than the action of another kinase.

The phosphorylation status of 4EBP1 regulates cap-dependent protein translation. Hypophosphorylated 4EBP1 binds to eIF4E and inhibits the formation of the eIF4F complex, while the phosphorylation of 4EBP1 inhibits its interaction with eIF4E. The ability of Torin1 to markedly inhibit 4EBP1 phosphorylation led us to examine the levels of the intact eIF4F complex in Torin1-treated cells. HCMV infection caused a decreased association of 4EBP1 with an analog of the m7G cap, m7GTP-Sepharose, throughout the course of infection (FIG. 4C), as was previously described (Walsh, et al. (2005) J. Virol. 79:8057-8064).

Rapamycin treatment did not increase the amount of 4EBP1 associated with the cap analog, consistent with its inability to block 4EBP1 phosphorylation during infection. In contrast, Torin1 treatment resulted in a substantially increased association of 4EBP1 with m7GTP-Sepharose throughout infection. HCMV infection did not alter the association of eIF4E with the cap analog, and this served as a loading control. In addition, the amount of tubulin in cell lysates was assayed to confirm that equal amounts of protein in each sample were loaded onto the cap analog. The increased level of 4EBP1 associated with m⁷GTPSepharose was consistent with the reduced association of eIF4G and eIF4A with the cap analog following Torin1 treatment (FIG. 4D). Rapamycin had minimal effects on the binding of eIF4G and eIF4A. Again, eIF4E levels were not affected by the drug and served as a loading control. These results indicate that the phosphorylation of 4EBP1 by rapamycin-resistant mTOR is required to maintain the integrity of the eIF4F complex during HCMV infection.

### Example 5

### Torin1 does not Block MCMV Replication in 4EBP1-Null Cells

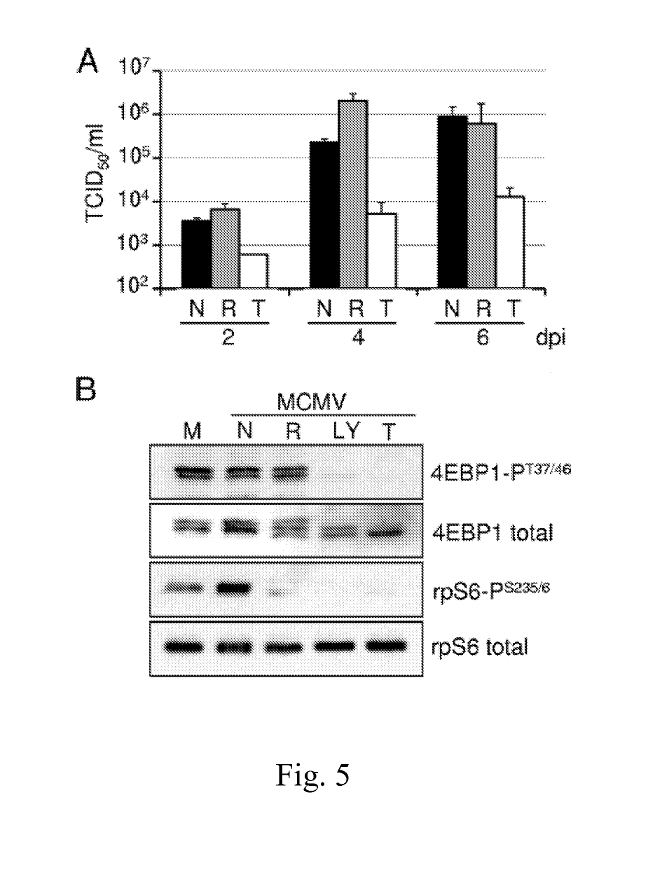

The identification of the functional roles of proteins in the mTOR signaling pathway has been facilitated by the generation of knockout mouse strains lacking individual mTOR components. For example, the availability of murine embryonic fibroblasts (MEFs) lacking the essential mTORC2 component Rictor led to the definitive identification of mTORC2 as the kinase complex responsible for the complete activation of Akt. We used murine cytomegalovirus (MCMV) and several MEF lines deficient for mTOR signaling pathway components to test for a possible contribution of mTORC2 to rapamycin-resistant phosphorylation events. To confirm that MCMV behaves like HCMV and is a suitable model for the analysis of mTOR signaling events, we determined the effect of Torin1 and rapamycin on MCMV growth and mTOR-dependent phosphorylation events in MEFs. As was the case for HCMV, Torin1, but not rapamycin, inhibited MCMV replication (FIG. 5A). Indeed, although rapamycin reduced the yield of HCMV to a modest extent (FIG. 1A), it had no inhibitory effect on MCMV. Also as observed for HCMV (FIG. 4A), MCMV infection induced mTORC1 activity, as measured by the increased phosphorylation of rpS6. The phosphorylation of rpS6 was completely inhibited by rapamycin, Torin1, and LY294002, an inhibitor of class 1 phosphatidylinositol 3-kinase and mTOR, whereas the phosphorylation of 4EBP1 was inhibited by Torin1 and LY294002 but not rapamycin (FIG. 5B). Total rpS6 protein was assayed and served as a loading control. Like HCMV, MCMV induces the mTOR signaling pathway, and it depends on rapamycin-resistant mTOR activity to induce the phosphorylation of 4EBP1.

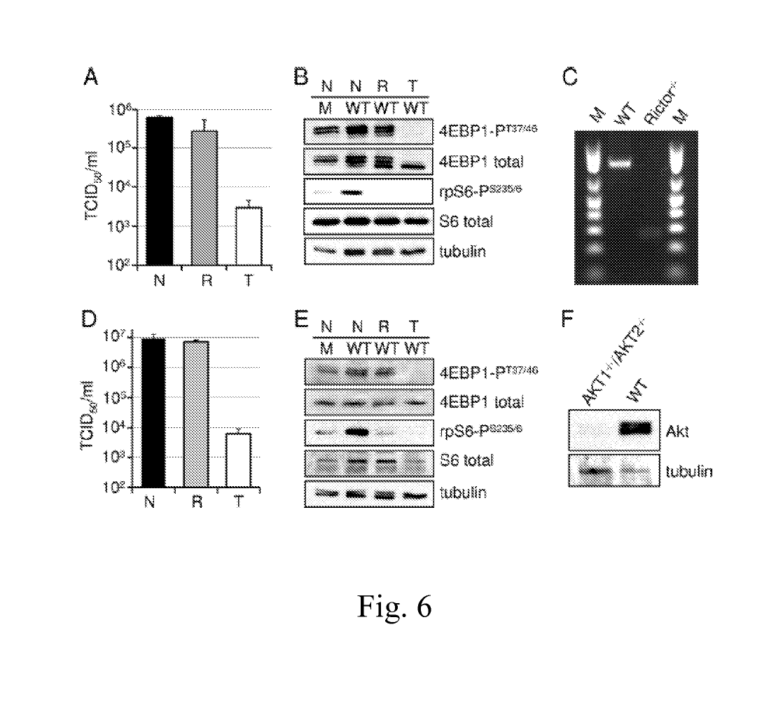

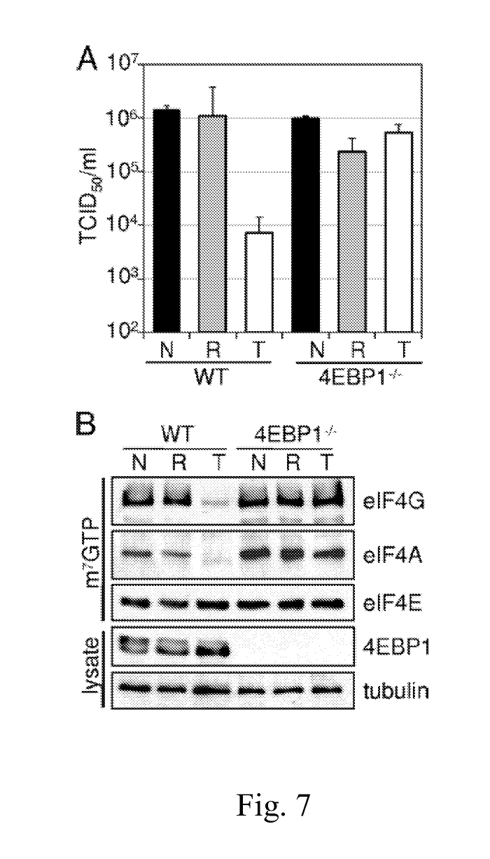

Having established that MCMV induces a set of mTOR signaling events similar to that of HCMV, the effect of Torin1 and rapamycin treatment on MEFs deficient for various effectors of mTOR action was characterized. We first investigated the requirement for mTORC2 for the replication of MCMV. Rictor-null MEFs supported viral growth (no treatment) (FIG. 6A), demonstrating that mTORC2 is not required for efficient MCMV replication. Furthermore, Torin1 effectively inhibited MCMV replication (FIG. 6A) and 4EBP1 phosphorylation (FIG. 6B) in these cells, arguing that mTORC2 is not the target for Torin1 in MCMV-infected cells. Finally, the cells were confirmed to lack an intact Rictor locus when assayed by PCR (FIG. 6C). We also employed Akt1/Akt2-null MEFs to evaluate a possible role for the Akt kinase, one of the targets of mTORC2. These cells supported Torin-sensitive MCMV replication (FIG. 6D), and Torin1 inhibited 4EBP1 phosphorylation in the absence of Akt (FIG. 6E), ruling out this kinase as the Torin1 target in MCMV-infected cells. Again, the cells were confirmed to lack Akt by Western blot assay (FIG. 6F). The inhibition of HCMV replication by Torin1 correlated with the hypophosphorylation of 4EBP1 (FIGS. 3 and 4), suggesting that this phosphorylation event might be the critical Torin1 target. Accordingly, we tested the ability of Torin1 to inhibit MCMV replication in 4EBP1-null MEFs (48). MCMV replicated as well in these cells as in normal MEFs, indicating that 4EBP1 is not required for cytomegalovirus replication (4EBP1^{-/-}, no treatment) (FIG. 7A). As in control cells, rapamycin had a minimal impact on MCMV replication in 4EBP1-null cells. Importantly, Torin1 was no longer capable of inhibiting MCMV replication in cells lacking 4EBP1 (FIG. 7A). 4EBP1 functions to inhibit eIF4F complex assembly unless inactivated by mTORC1-mediated phosphorylation. While Torin1 treatment inhibited the formation of the eIF4F complex in control cells, no such effect was observed for 4EBP1-null cells (FIG. 7B). Finally, that 4EBP1 was not detected in lysates of these cells by Western blot assay confirmed the phenotype of the MEFs. We conclude that 4EBP1 is a target providing sensitivity to Torin1 during cytomegalovirus infection, and we propose that rapamycin-resistant mTORC1 is required for the maintenance of cap-dependent translation during the viral life cycle.

### Example 6

### Members of all Three Herpesvirus Subfamilies are Inhibited by Torin1

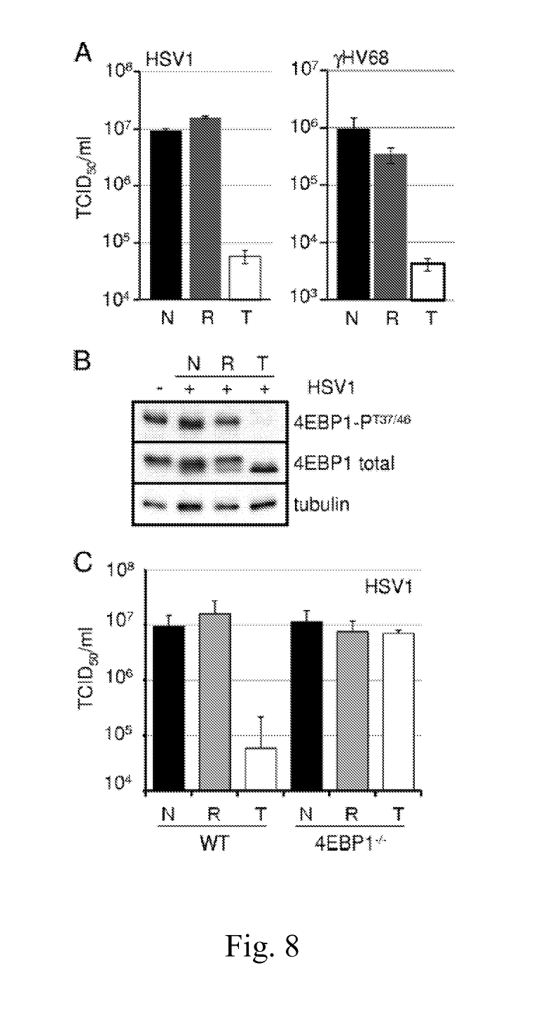

MEFs were infected with the alphaherpesvirus, herpes simplex virus type 1 (HSV-1), and the gammaherpesvirus, murine gammaherpesvirus 68 (γHV68) (FIG. 8A). These viruses exhibited the same drug sensitivities as the cytomegaloviruses. While rapamycin was ineffective at preventing HSV-1 and γHV68 replication, Torin1 inhibited both viruses over multiple rounds of viral replication. In addition, Torin1, but not rapamycin, inhibited the phosphorylation of 4EBP1 during HSV-1 infection (FIG. 8B), and Torin1 failed to inhibit the production of HSV-1 in cells lacking 4EBP1 (FIG. 8C). We conclude that rapamycin-resistant mTOR activity is required for the replication of multiple herpesviruses.

### Example 7

### Inhibition of HCMV Yield by Treatment of Fibroblasts with siRNA Directed Against the MTOR Kinase

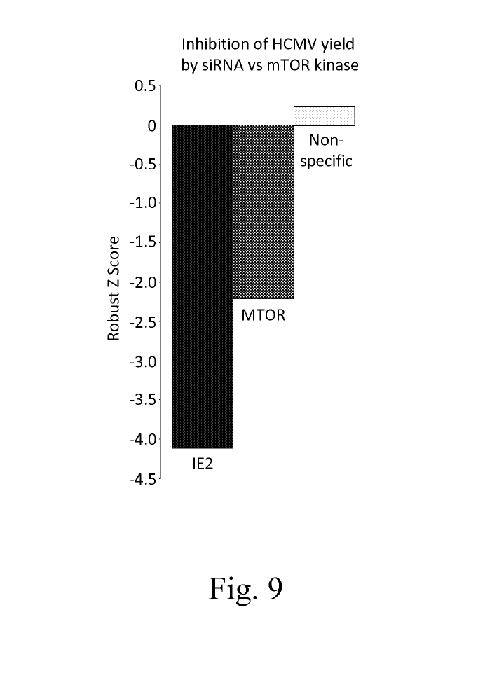

MRC5 fibroblasts (ATCC #CCL-171) at passage 23-24 were plated at a density of 7500 cells/well in DMEM (Sigma-Aldrich product #D5756, St. Louis, Mo.) supplemented 10% FBS (GIBCO) in 96-well plastic tissue culture dishes (TRP#92696, Switzerland). Cells were grown to ˜70% confluence and then transfected with 1 nmol siRNA targeting GFP mRNA (non-specific), the viral IE2 mRNA, or mTOR kinase using Oligofectamine (Invitrogen, Carlsbad, Calif.) per manufacturer's instructions. IE2 siRNA sequence: 5′-AAACGCAUCUCCGAGUUGGAC-3′ (SEQ ID NO:1); GFP siRNA sequence: 5′-GCAAGCUGACCCUGAAGUUCAU-3′ (SEQ ID NO:2); mTOR kinase (FRAP1_{—}2) siRNA sequence: 5′-GAGUUACAGUCGGGCAUAU-3′ (SEQ ID NO:3). All siRNAs were obtained from Sigma-Aldrich. 4 h post-transfection, medium was supplemented with FBS to 10% final concentration. 28 h post-transfection, culture supernatants were removed and replaced with 1001 DMEM/10% FBS containing HCMV strain AD169 at a concentration of 0.1 pfu per cell. Infection proceeded for 96 h, at which time culture supernatants were harvested and used to infect a fresh plate of −90% confluent MRC5 cells in 96-well format. 24 h post-infection of this reporter plate, the samples were fixed with chilled methanol at −20° for 15 min and processed for immunofluorescence to quantify infectivity. Results in FIG. 9 are presented as “robust Z score”, which correlates with standard deviations from mean value for infectivity generated in the absence of siRNA treatment. Thus, the mTOR kinase-specific siRNA reduced the yield of infectious HCMV by a factor of >2 standard deviations, a highly significant effect.

### Example 8

### Inhibition of HCMV Yield by Treatment of Fibroblasts with an Inhibitor of the Unfolded Protein Response

To explore the hypothesis that HCMV might actually require the UPR to occur in order to maintain cellular homeostasis despite high levels of expression of viral glycoproteins, HCMV-infected human fibroblasts (HFFs) were treated with an inhibitor of the UPR, the chemical chaperone sodium 4-phenylbutyrate (4-PBA). Treatment with 4-PBA effectively inhibited virus replication in a dose-dependent manner (FIG. 10).

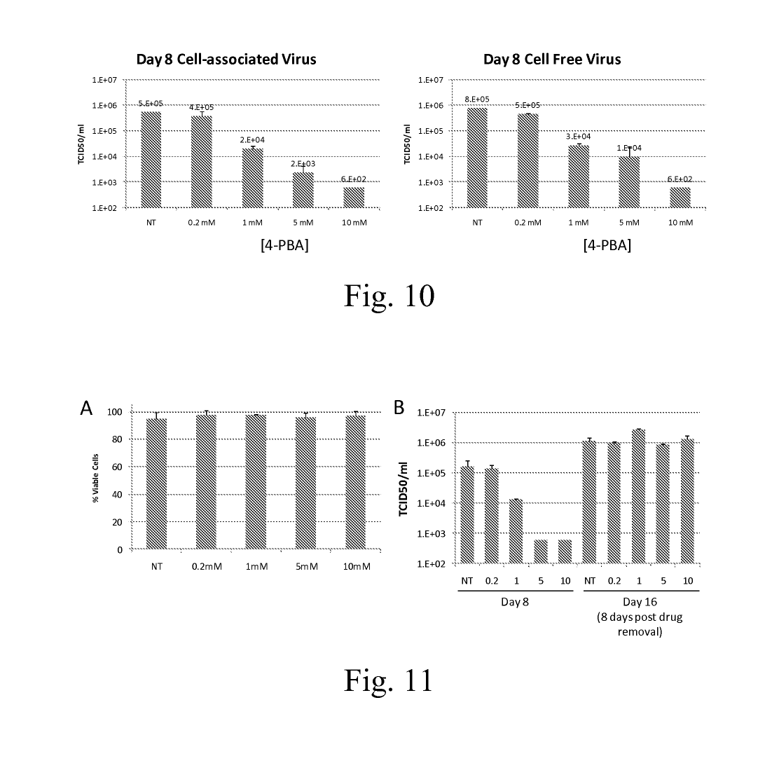

Two experiments were performed to rule out the possibility that 4-PBA is simply toxic to the cells and inhibits HCMV indirectly by reducing cell viability (FIG. 11). In the first experiment (FIG. 11A) an assay for cell viability was performed on confluent human fibroblasts treated for eight days with different concentrations of the drug. The highest dose of the drug tested had no effect on cellular viability in the trypan blue exclusion assay. In the second experiment, the drug was shown to be reversible (FIG. 11B). Infected cells were maintained in the presence of different concentrations for the drug for 8 days, and a sample was taken to determine the yield of virus. As in the previous experiment (FIG. 10), the drug inhibited virus production in a dose-dependent manner. Then the drug was removed and the yield of virus was determined 8 days later. For all doses of drug tested, the virus recovered and produced a normal yield. This shows that the drug did not damage the cell during an 8-day treatment, because the cell remained capable of producing a normal virus yield.

This demonstrates that HCMV depends on the UPR to produce a normal yield of infectious progeny. Importantly, this data also demonstrates that a drug which inhibits the UPR acts as an anti-HCMV therapeutic. Drugs that inhibit the UPR are also predicted have antiviral properties towards other herpesviruses and other viruses as well, based upon the high levels of viral glycoproteins expressed during infection by many viruses. Drugs in this class include 4-PBA as well as Tauroursodeoxycholic acid (TUDCA). 4-PBA is currently used clinically for the treatment of urea cycle disorders in newborns. Serum concentrations similar to those used in this study have been measured in patients treated with 4-PBA. This demonstrates that 4-PBA is safe and well tolerated in individuals with poorly functioning immune systems, the same patient groups which suffer from cytomegalovirus disease, and that a dose of 4-PBA that inhibits HCMV replication can be achieved in vivo.

### Example 9

### Inhibition of HCMV Yield by Treatment of Fibroblasts with a Combination of an MTOR Inhibitor and an Inhibitor of the Unfolded Protein Response

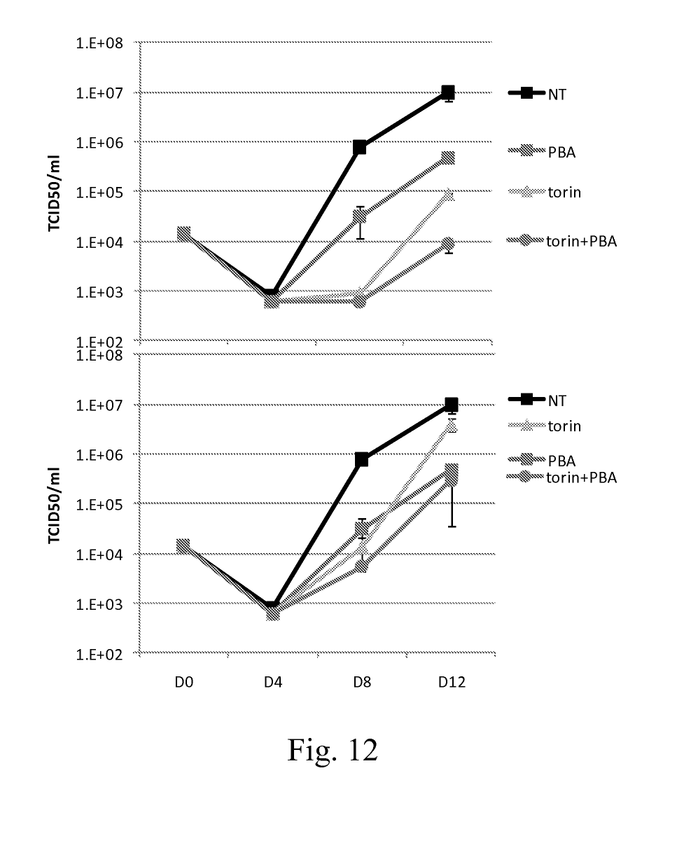

Torin1 when combined with 4-PBA inhibited HCMV to a greater extent than either drug alone, and 4-PBA plus rapamycin also inhibited HCMV to a greater extent than either drug alone (FIG. 12). Human fibroblasts were infected with HCMV strain AD169 at a multiplicity of 0.1 pfu/cell and maintained in medium containing 10% fetal calf serum and either rapamycin or Torin1 alone and in combination with 4-PBA at the following concentrations: 4-PBA, 1 mM; Torin1, 250 nM; rapamycin, 20 nM. The medium with drug(s) was replaced every other day. Cell-free and cell-associated virus was collected on days 0, 4, 8 and 12 post infection, and titered by the TCID₅₀ method.

### Example 10

### Dose-Dependent Inhibition of Human Cytomegalovirus Replication by MTOR Inhibitors

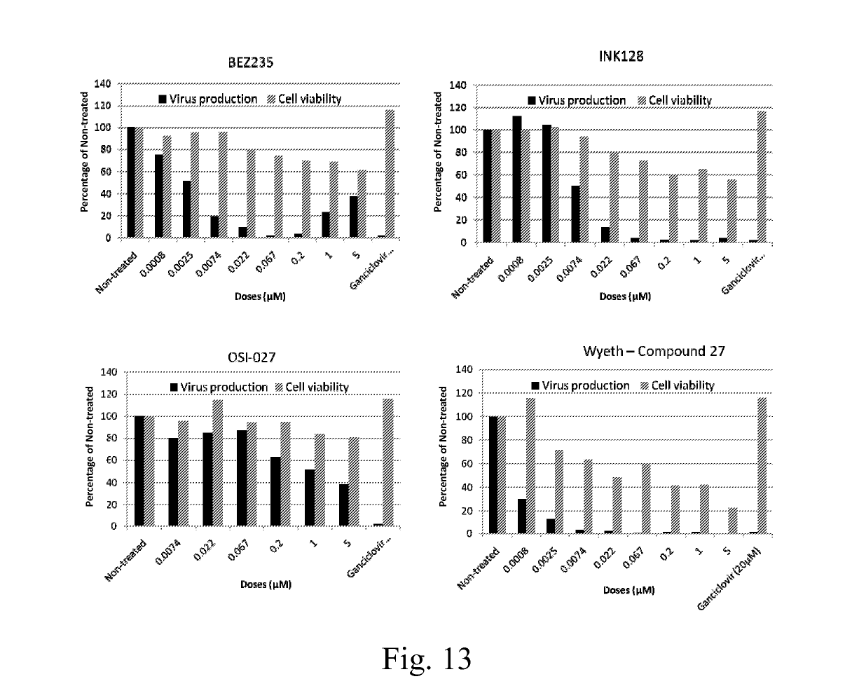

MRC5 human fibroblasts at about 95% confluency were infected with HCMV at a multiplicity of 0.5 pfu/cell. Two hours after infection, the medium of the cells was replaced with fresh medium containing either BEZ235 (at concentrations from 0.0008 μM to 5 μM); INK128 (at concentrations from 0.0008 μM to 5 μM); OSI-027 (at concentrations from 0.0074 μM to 5 μM); and Wyeth—Compound 27 (Wyeth-BMCL20102648-27) (at concentrations from 0.0008 μM to 5 μM). The yield of HCMV was determined at 96 hours post-infection and represented as the percentage of virus yield in untreated cells. Ganciclovir (a marketed drug for HCMV) was used as comparator for the efficacy of tested compounds in the assay. The effects of the compounds on the viability of uninfected MRC5 cells at 96 hours of treatment were assessed by using the Toxilight bioassay kit (Lonza). The results, shown in FIG. 13, demonstrate that mTOR inhibitors with unrelated structures inhibit virus production.

1. A method of treating or preventing viral infection in a mammal, comprising administering to a mammalian subject in need thereof a therapeutically effective amount of a compound or prodrug thereof, or pharmaceutically acceptable salt or ester of said compound or prodrug, wherein the compound is an inhibitor of a rapamycin-resistant function of mTOR and wherein the compound is a compound of Formula VIII 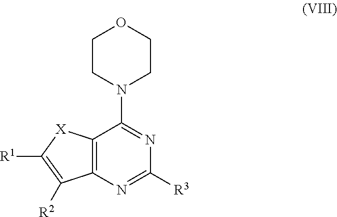
wherein:
X is O or S;
R¹ is selected from H, F, Cl, Br, I, CN, —CR¹⁴R¹⁵—NR¹⁶R¹⁷, —CR¹⁴R¹⁵—NHR¹⁰, —(CR¹⁴R¹⁵)ₜNR¹⁰R¹¹, —C(R¹⁴R¹⁵)ₙNR¹²C(═Y)R¹⁰, —(CR¹⁴R¹⁵)ₙNR¹²S(O)₂R¹⁰, —(CR¹⁴R¹⁵)ₘOR¹⁰, —(CR¹⁴R¹⁵)ₙS(O)₂R¹⁰, —(CR¹⁴R¹⁵)ₙS(O)₂NR¹⁰R¹¹, —C(OR¹⁰)R¹¹R¹⁴, —C(R¹⁴)═CR¹⁸R¹⁹, —C(═Y)R¹⁰, —C(═Y)OR¹⁰, —C(═Y)NR¹⁰R¹¹, —C(═Y)NR¹²OR¹⁰, —C(═O)NR¹²S(O)₂R¹⁰, —C(═O)NR¹²(CR¹⁴R¹⁵)ₘNR¹⁰R¹¹, —NO₂, —NHR¹², —NR¹²C(═Y)R¹¹, —NR¹²C(═Y)OR¹¹, —NR¹²C(═Y)NR¹⁰R¹¹, —NR¹²S(O)₂R¹⁰, —NR¹²SO₂NR¹⁰R¹¹, —S(O)₂R¹⁰, —S(O)₂NR¹⁰R¹¹, —SC(═Y)R¹⁰, —SC(═Y)OR¹⁰, C₂-C₁₂ alkyl, C₂-C₁₂ alkyl-R¹⁰, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl;
R² is selected from H, F, Cl, Br, I, CN, CF₃, —NO₂, —C(═Y)R¹⁰, —C(—Y)OR¹⁰, —C(═Y)NR¹⁰R¹¹, —(CR¹⁴R¹⁵)ₘNR¹⁰R¹¹, —(CR¹⁴R¹⁵)ₙOR¹⁰, —(CR¹⁴R¹⁵)ₜ—NR¹²C(═O)(CR¹⁴R¹⁵)NR¹⁰R¹¹, —NR¹²C(═Y)R¹⁰, —NR¹²C(═Y)OR¹⁰, —NR¹²C(═Y)NR¹⁰R¹¹, —NR¹²SO²R¹⁰, OR¹⁰, —OC(═Y)R¹⁰, —OC(═Y)OR¹⁰, —OC(═Y)NR¹⁰R¹¹, —OS(O)₂(OR¹⁰), —OP(═Y)(OR¹⁰)(OR¹¹), —OP(OR¹⁰)(OR¹¹), SR¹⁰, —S(O)R¹⁰, —S(O)₂R¹⁰, —S(O)₂NR¹⁰R¹¹, —S(O)(OR¹⁰), —S(O)₂(OR¹⁰), —SC(═Y)R¹⁰, —SC(═Y)OR¹⁰, —SC(═Y)NR¹⁰R¹¹, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, and C₁-C₂₀ heteroaryl;
R³ is a C₂-C₅ heterocyclyl, a C₂-C₅ heteroaryl, a fused bicyclic C₄-C₂₀ heterocyclyl or a fused bicyclic C₃-C₂₀ heteroaryl, each of which are unsubstituted or are optionally substituted;
R¹⁰, R¹¹ and R¹² are independently H, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl,
or R¹⁰ and R¹¹ together with the nitrogen to which they are attached optionally form a saturated, partially unsaturated or fully unsaturated C₃-C₂₀ heterocyclic ring optionally containing one or more additional ring atoms selected from N, O or S, wherein said heterocyclic ring is optionally substituted with one or more groups independently selected from oxo, (CH₂)ₘOR¹⁰NR¹⁰R¹¹, CF₃, F, Cl, Br, I, SO₂R¹⁰, C(═O)R¹⁰, NR¹²C(═Y)R¹¹, NR¹²S(O)₂R¹¹, C(═Y)NR¹⁰R¹¹, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl and C₁-C₂₀ heteroaryl;
R¹⁴ and R¹⁵ are independently selected from H, C₁-C₁₂ alkyl, or —(CH₂)-aryl,
or R¹⁴ and R¹⁵ together with the atoms to which they are attached form a saturated or partially unsaturated C₃-C₁₂ carbocyclic ring,
R¹⁶ and R¹⁷ are independently H, C₁-C₁₂ alkyl, C₂-C₈ alkenyl, C₂-C₈ alkynyl, C₃-C₁₂ carbocyclyl, or C₆-C₂₀ aryl,
R¹⁸ and R¹⁹ together with the carbon to which they are attached form a C₃-C₂₀ heterocyclic ring,
where said alkyl, alkenyl, alkynyl, carbocyclyl, heterocyclyl, aryl and heteroaryl are optionally substituted with one or more groups independently selected from F, Cl, Br, I, CN, CF₃, —NO₂, oxo, R¹⁰, —C(═Y)R¹⁰, —C(═Y)OR¹⁰, —C(═Y)NR¹⁰R¹¹, —(CR¹⁴R¹⁵)ₙNR¹⁰R¹¹, —(CR¹⁴R¹⁵)ₙOR¹⁰, —NR¹⁰R¹¹, —NR¹²C(═Y)R¹⁰, —NR¹²C(═Y)OR¹¹, —NR¹²C(═Y)NR¹⁰R¹¹, —NR¹²SO₂R¹⁰, ═NR¹², OR¹⁰, —OC(═Y)R¹⁰, —OC(═Y)OR¹⁰, —OC(═Y)NR¹⁰R¹¹, —OS(O)₂(OR¹⁰), —OP(═Y)(OR¹⁰)(OR¹¹), —OP(OR¹⁰)(OR¹¹), SR¹⁰, —S(O)R¹⁰, —S(O)₂R¹⁰, —S(O)₂NR¹⁰R¹¹, —S(O)(OR¹⁰), —S(O)₂(OR¹⁰), —SC(═Y)R¹⁰, —SC(═Y)OR¹⁰, —SC(═Y)NR¹⁰R¹¹, C₁-C₁₂ optionally substituted alkyl, C₂-C₈ optionally substituted alkenyl, C₂-C₈ optionally substituted alkynyl, C₃-C₁₂ optionally substituted carbocyclyl, C₂-C₂₀ optionally substituted heterocyclyl, C₆-C₂₀ optionally substituted aryl, C₁-C₂₀ optionally substituted heteroaryl, —(CR¹⁴R¹⁵)ₜ—NR¹²C(═O)(CR¹⁴R¹⁵)NR¹⁰R¹¹, and (CR¹⁴R¹⁵)ₜNR¹⁰R¹¹;
Y is O, S, or NR¹²;
m is 0, 1, 2, 3, 4, 5 or 6;
n is 1, 2, 3, 4, 5 or 6; and
t is 1, 2, 3, 4, 5 or 6, and
wherein the substituent groups that are alkyls, alkenyls, alkynyls, carbocyclyls, heterocyclyls, aryls, heteroaryls, fused bicyclic heterocyclyls, and fused bicyclic heteroaryls are optionally substituted with one or more substituents selected from the group consisting of F, Cl, Br, I, CN, CF₃, —NO₂, —NH₂, oxo, R¹⁰, —C(═Y)R¹⁰, —C(═Y)OR¹⁰, —C(═Y)NR¹⁰R¹¹, —(CR¹⁴R¹⁵)ₙNR¹⁰R¹¹, —(CR¹⁴R¹⁵)ₙOR¹⁰, —NR¹⁰R¹¹, —NR¹²C(═Y)R¹⁰, —NR¹²C(═Y)OR¹¹, —NR¹²C(═Y)NR¹⁰R¹¹, —NR¹²SO₂R¹⁰, ═NR¹², OR¹⁰, —OC(═Y)R¹⁰, —OC(═Y)OR¹⁰, —OC(═Y)NR¹⁰R¹¹, —OS(O)₂(OR¹⁰), —OP(═Y)(OR¹⁰)(OR¹¹), —OP(OR¹⁰)(OR¹¹), SR¹⁰, —S(O)R¹⁰, —S(O)₂R¹⁰, —S(O)₂NR¹⁰R¹¹, —S(O)(OR¹⁰), —S(O)₂(OR¹⁰), —SC(═Y)R¹⁰, —SC(═Y)OR¹⁰, —SC(═Y)NR¹⁰R¹¹, C₁-C₁₂ optionally substituted alkyl, C₂-C₈ optionally substituted alkenyl, C₂-C₈ optionally substituted alkynyl, C₃-C₁₂ optionally substituted carbocyclyl, C₂-C₂₀ optionally substituted heterocyclyl, C₆-C₂₀ optionally substituted aryl, C₁-C₂₀ optionally substituted heteroaryl, —(CR¹⁴R¹⁵)ₜ—NR¹²C(═O)(CR¹⁴R¹⁵)NR¹⁰R¹¹, and (CR¹⁴R¹⁵)ₜ—NR¹⁰R¹¹.

2. The method of claim 1, wherein the compound of formula VIII is GNE-493.

3. The method of claim 1, wherein the compound of formula VIII is GNE-0941.

4. The method of claim 1, wherein the compound is an inhibitor of mTORC1.

5. The method of claim 1, wherein the compound is an inhibitor of mTORC2.

6. The method of claim 1, wherein the viral infection is by a herpesvirus.

7. The method of claim 1, wherein the viral infection is by a herpesvirus selected from the group consisting of herpes simplex virus type 1, herpes simplex virus type 2, varicella-zoster virus, human cytomegalovirus, Epstein-Barr virus, human herpesvirus 6 variant A, human herpesvirus 6 variant B, human herpesvirus 7, human herpesvirus 8, and cercopithecine herpesvirus 1.

8. The method of claim 1, further comprising administering to the mammalian subject an inhibitor of the unfolded protein response.

9. The method of claim 8 wherein the inhibitor of the unfolded protein response is 4-phenylbutyrate.

10. The method of claim 8 wherein the inhibitor of the unfolded protein response is tauroursodeoxycholic acid.

11. A pharmaceutical composition for treatment or prevention of a viral infection comprising a therapeutically effective amount of a composition comprising (i) a compound or prodrug thereof, or pharmaceutically acceptable salt of said compound or prodrug; and (ii) a pharmaceutically acceptable carrier, wherein the compound is an inhibitor of a rapamycin-resistant function of mTOR and wherein the compound is a compound of Formula VIII: 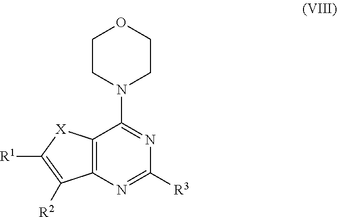
wherein:
X is O or S;
R¹ is selected from H, F, Cl, Br, I, CN, —CR¹⁴R¹⁵—NR¹⁶R¹⁷, —CR¹⁴R¹⁵—NHR¹⁰, —(CR¹⁴R¹⁵)ₜNR¹⁰R¹¹, —C(R¹⁴R¹⁵)ₙNR¹²C(═Y)R¹⁰, —(CR¹⁴R¹⁵)NR¹²S(O)₂R¹⁰, —(CR¹⁴R¹⁵)ₘOR¹⁰, —(CR¹⁴R¹⁵)S(O)₂R¹⁰, —(CR¹⁴R¹⁵)ₙS(O)₂NR¹⁰R¹¹, —C(OR¹⁰)R¹¹R¹⁴, —C(R¹⁴)═CR¹⁸R¹⁹, —C(═Y)R¹⁰, —C(═Y)OR¹⁰, —C(═Y)NR¹⁰R¹¹, —C(═Y)NR¹²OR¹⁰, —C(═O)NR¹²S(O)₂R¹⁰, —C(═O)NR¹²(CR¹⁴R¹⁵)ₘNR¹⁰R¹¹, —NO₂, —NHR¹², —NR¹²C(═Y)R¹¹, —NR¹²C(═Y)OR¹¹, —NR¹²C(═Y)NR¹⁰R¹¹, —NR¹²S(O)₂R¹⁰, —NR¹²SO₂NR¹⁰R¹¹, —S(O)₂R¹⁰, —S(O)₂NR¹⁰R¹¹, —SC(═Y)R¹⁰, —SC(═Y)OR¹⁰, C₂-C₁₂ alkyl, C₂-C₁₂ alkyl-R¹⁰, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl;
R² is selected from H, F, Cl, Br, I, CN, CF₃, —NO₂, —C(═Y)R¹⁰, —C(—Y)OR¹⁰, —C(═Y)NR¹⁰R¹¹, —(CR¹⁴R¹⁵)ₘNR¹⁰R¹¹, —(CR¹⁴R¹⁵)ₙOR¹⁰, —(CR¹⁴R¹⁵)ₜNR¹²C(═O)(CR¹⁴R¹⁵)NR¹⁰R¹¹, —NR¹²C(═Y)R¹⁰, —NR¹²C(═Y)OR¹⁰, —NR¹²C(═Y)NR¹⁰R¹¹, —NR¹²SO₂R¹⁰, OR¹⁰, —OC(═Y)R¹⁰, —OC(═Y)OR¹⁰, —OC(═Y)NR¹⁰R¹¹, —OS(O)₂(OR¹⁰), —OP(═Y)(OR¹⁰)(OR¹¹), —OP(OR¹⁰)(OR¹¹), SR¹⁰, —S(O)R¹⁰, —S(O)₂R¹⁰, —S(O)₂NR¹⁰R¹¹, —S(O)(OR¹⁰), —S(O)₂(OR¹⁰), —SC(═Y)R¹⁰, —SC(═Y)OR¹⁰, —SC(═Y)NR¹⁰R¹¹, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, and C₁-C₂₀ heteroaryl;
R³ is a C₂-C₅ heterocyclyl, a C₂-C₅ heteroaryl, a fused bicyclic C₄-C₂₀ heterocyclyl or a fused bicyclic C₃-C₂₀ heteroaryl, each of which are unsubstituted or are optionally substituted;
R¹⁰, R¹¹ and R¹² are independently H, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl,
or R¹⁰ and R¹¹ together with the nitrogen to which they are attached optionally form a saturated, partially unsaturated or fully unsaturated C₃-C₂₀ heterocyclic ring optionally containing one or more additional ring atoms selected from N, O or S, wherein said heterocyclic ring is optionally substituted with one or more groups independently selected from oxo, (CH₂)ₘOR¹⁰NR¹⁰R¹¹, CF₃, F, Cl, Br, I, SO₂R¹⁰, C(═O)R¹⁰, NR¹²C(═Y)R¹¹, NR¹²S(O)₂R¹¹, C(═Y)NR¹⁰R¹¹, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl and C₁-C₂₀ heteroaryl;
R¹⁴ and R¹⁵ are independently selected from H, C₁-C₁₂ alkyl, or —(CH₂)ₙ-aryl,
or R¹⁴ and R¹⁵ together with the atoms to which they are attached form a saturated or partially unsaturated C₃-C₁₂ carbocyclic ring,
R¹⁶ and R¹⁷ are independently H, C₁-C₁₂ alkyl, C₂-C₈ alkenyl, C₂-C₈ alkynyl, C₃-C₁₂ carbocyclyl, or C₆-C₂₀ aryl,
R¹⁸ and R¹⁹ together with the carbon to which they are attached form a C₃-C₂₀ heterocyclic ring,
where said alkyl, alkenyl, alkynyl, carbocyclyl, heterocyclyl, aryl and heteroaryl are optionally substituted with one or more groups independently selected from F, Cl, Br, I, CN, CF₃, —NO₂, oxo, R¹⁰, —C(═Y)R¹⁰, —C(═Y)OR¹⁰, —C(═Y)NR¹⁰R¹¹, —(CR¹⁴R¹⁵)ₙNR¹⁰R¹¹, —(CR¹⁴R¹⁵)ₙOR¹⁰, —NR¹⁰R¹¹, —NR¹²C(═Y)R¹⁰, —NR¹²C(═Y)OR¹¹, —NR¹²C(═Y)NR¹⁰R¹¹, —NR¹²SO₂R¹⁰, ═NR¹², OR¹⁰, —OC(═Y)R¹⁰, —OC(═Y)OR¹⁰, —OC(═Y)NR¹⁰R¹¹, —OS(O)₂(OR¹⁰), —OP(═Y)(OR¹⁰)(OR¹¹), —OP(OR¹⁰)(OR¹¹), SR¹⁰, —S(O)R¹⁰, —S(O)₂R¹⁰, —S(O)₂NR¹⁰R¹¹, —S(O)(OR¹⁰), —S(O)₂(OR¹⁰), —SC(═Y)R¹⁰, —SC(═Y)OR¹⁰, —SC(═Y)NR¹⁰R¹¹, C₁-C₁₂ optionally substituted alkyl, C₂-C₈ optionally substituted alkenyl, C₂-C₈ optionally substituted alkynyl, C₃-C₁₂ optionally substituted carbocyclyl, C₂-C₂₀ optionally substituted heterocyclyl, C₆-C₂₀ optionally substituted aryl, C₁-C₂₀ optionally substituted heteroaryl, —(CR¹⁴R¹⁵)ₜ—NR¹²C(═O)(CR¹⁴R¹⁵)NR¹⁰R¹¹, and (CR¹⁴R¹⁵)NR¹⁰R¹¹;
Y is O, S, or NR¹²;
m is 0, 1, 2, 3, 4, 5 or 6;
n is 1, 2, 3, 4, 5 or 6; and
t is 1, 2, 3, 4, 5 or 6, and
wherein the substituent groups that are alkyls, alkenyls, alkynyls, carbocyclyls, heterocyclyls, aryls, heteroaryls, fused bicyclic heterocyclyls, and fused bicyclic heteroaryls are optionally substituted with one or more substituents selected from the group consisting of F, Cl, Br, I, CN, CF₃, —NO₂, —NH₂, oxo, R¹⁰, —C(═Y)R¹⁰, —C(═Y)OR¹⁰, —C(═Y)NR¹⁰R¹¹, —(CR¹⁴R¹⁵)ₙNR¹⁰R¹¹, —(CR¹⁴R¹⁵)OR¹⁰, —NR¹⁰R¹¹, —NR¹²C(═Y)R¹⁰, —NR¹²C(═Y)OR¹¹, —NR¹²C(═Y)NR¹⁰R¹¹, —NR¹²SO₂R¹⁰, ═NR¹², OR¹⁰, —OC(═Y)R¹⁰, —OC(═Y)OR¹⁰, —OC(═Y)NR¹⁰R¹¹, —OS(O)₂(OR¹⁰), —OP(═Y)(OR¹⁰)(OR¹¹), —OP(OR¹⁰)(OR¹¹), SR¹⁰, —S(O)R¹⁰, —S(O)₂R¹⁰, —S(O)₂NR¹⁰R¹¹, —S(O)(OR¹⁰), —S(O)₂(OR¹⁰), —SC(═Y)R¹⁰, —SC(═Y)OR¹⁰, —SC(═Y)NR¹⁰R¹¹, C₁-C₁₂ optionally substituted alkyl, C₂-C₈ optionally substituted alkenyl, C₂-C₈ optionally substituted alkynyl, C₃-C₁₂ optionally substituted carbocyclyl, C₂-C₂₀ optionally substituted heterocyclyl, C₆-C₂₀ optionally substituted aryl, C₁-C₂₀ optionally substituted heteroaryl, —(CR¹⁴R¹⁵)ₜ—NR¹²C(═O)(CR¹⁴R¹⁵)NR¹⁰R¹¹, and (CR¹⁴R¹⁵)ₜ—NR¹⁰R¹¹.

12. The pharmaceutical composition of claim 11, wherein the compound of formula VIII is GNE-493.

13. The pharmaceutical composition of claim 11, wherein the compound of formula VIII is GNE-0941.

14. The pharmaceutical composition of claim 11, wherein the compound is an inhibitor of mTORC1.

15. The pharmaceutical composition of claim 11, wherein the compound is an inhibitor of mTORC2.

16. The pharmaceutical composition of claim 11, wherein the viral infection is by a herpesvirus.

17. The pharmaceutical composition of claim 11, wherein the viral infection is by a herpesvirus selected from the group consisting of herpes simplex virus type 1, herpes simplex virus type 2, varicella-zoster virus, human cytomegalovirus, Epstein-Barr virus, human herpesvirus 6 variant A, human herpesvirus 6 variant B, human herpesvirus 7, human herpesvirus 8, and cercopithecine herpesvirus 1.

18. The pharmaceutical composition of claim 11, further comprising administering to the mammalian subject an inhibitor of the unfolded protein response.

19. The pharmaceutical composition of claim 18 wherein the inhibitor of the unfolded protein response is 4-phenylbutyrate.

20. The pharmaceutical composition of claim 18 wherein the inhibitor of the unfolded protein response is tauroursodeoxycholic acid.

21-40. (canceled)

41. A method of treating or preventing viral infection in a mammal, comprising administering to a mammalian subject in need thereof a therapeutically effective amount of a compound or prodrug thereof, or pharmaceutically acceptable salt or ester of said compound or prodrug, wherein the compound is an inhibitor of a rapamycin-resistant function of mTOR and wherein the compound is a compound of Formula IX 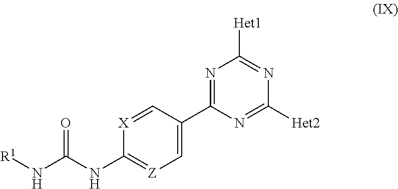
wherein,
Het1 and Het2 are independently selected from (i) a 5 to 7 membered heterocyclic group having 1-3 heteroatoms selected from O, N, and S, and (ii) a 6 to 10 membered bicyclic heterocyclic group having 1-3 heteroatoms selected from O, N, and S; wherein each of (i) or (ii) may by unsubstituted or substituted with 1 to 3 substituents selected from C₁-C₄ alkyl, NH₂, —O—R⁰, CN, and halo;
X and Z are independently selected from N and CH;
R¹ is selected from the group consisting of:
(i) C₁-C₁₀ alkyl, C₂-C₁₀ alkenyl, C₂-C₁₀ alkynyl, and C₃ to C₁₀ cycloalkyl; each of which is unsubstituted or is substituted by 1 to 3 substituents selected from the group consisting of halo, —N(R⁰)₂, CN, NO₂, —O—(C₂-C₁₀ alkyl), aryl, heteroaryl, and heterocyclyl;
(ii) aryl, heteroaryl and heterocyclyl; each of which is unsubstituted or is substituted by 1 to 3 substituents selected from the group consisting of halo, —N(R⁰)₂, CN, NO₂, C₁-C₆ alkyl, C₂-C₆ alkenyl, aryl, heteroaryl, heterocyclyl, —O—R⁰, —(C₁-C₁₀ alkyl)-OR⁰, —O—(C₁-C₁₀ alkyl)-OR⁰, —(C₁-C₁₀ alkyl)-N(R⁰)₂, —O—(C₂-C₁₀ alkyl)-N(R⁰)₂, —O—(C₂-C₁₀ alkyl)-C(═O)—N(R⁰)₂, —C(═O)—(C₂-C₁₀ alkyl)-N(R⁰)₂, —C(═O)N(R⁰)—(C₂-C₁₀ alkyl)-N(R⁰)₂, and —(C₁-C₆ alkyl)-aryl;
(iii) -aryl-C(═O)-aryl, -aryl-C(═O)-heteroaryl, and -aryl-C(═O)-heterocyclyl; each of which is unsubstituted or is substituted by 1 to 3 substituents selected from the group consisting of halo, —N(R⁰)₂, CN, NO₂, C₁-C₆ alkyl, C₂-C₆ alkenyl, and —OR⁰; and
(iv) -aryl-aryl, -aryl-heteroaryl, and -aryl-heterocyclyl; each of which is unsubstituted or is substituted by 1 to 3 substituents selected from the group consisting of halo, —N(R⁰)₂, CN, NO₂, C₁-C₆ alkyl, C₂-C₆ alkenyl, —O—R⁰; and
each R⁰ is independently selected from H and C₁-C₆ alkyl; or alternatively two adjacent R⁰ attached to a nitrogen atom may be taken together to form a 5 or 6-membered heterocyclic ring having 0 to 2 additional ring heteroatoms selected from N, O, and S, and which may be unsubstituted or substituted by 1 to 3 substituents selected form the group consisting of C₁-C₄ alkyl, NH₂, —OH, CN, and halo.

42. The method of claim 41, wherein the compound of Formula IX is Wyeth BMCL 20102644-13.

43. The method of claim 41, wherein the compound of Formula IX is Wyeth BMCL 20102648-27.

44. The method of claim 41, wherein the compound is an inhibitor of mTORC1.

45. The method of claim 41, wherein the compound is an inhibitor of mTORC2.

46. The method of claim 41, wherein the viral infection is by a herpesvirus.

47. The method of claim 41, wherein the viral infection is by a herpesvirus selected from the group consisting of herpes simplex virus type 1, herpes simplex virus type 2, varicella-zoster virus, human cytomegalovirus, Epstein-Barr virus, human herpesvirus 6 variant A, human herpesvirus 6 variant B, human herpesvirus 7, human herpesvirus 8, and cercopithecine herpesvirus 1.

48. The method of claim 41, further comprising administering to the mammalian subject an inhibitor of the unfolded protein response.

49. The method of claim 48, wherein the inhibitor of the unfolded protein response is 4-phenylbutyrate.

50. The method of claim 48, wherein the inhibitor of the unfolded protein response is tauroursodeoxycholic acid.

51. A pharmaceutical composition for treatment or prevention of a viral infection comprising a therapeutically effective amount of a composition comprising (i) a compound or prodrug thereof, or pharmaceutically acceptable salt of said compound or prodrug; and (ii) a pharmaceutically acceptable carrier, wherein the compound is an inhibitor of a rapamycin-resistant function of mTOR and wherein the compound is a compound of Formula IX 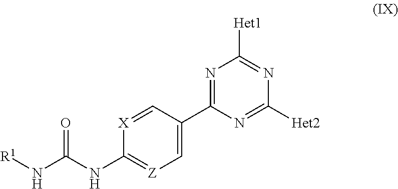
wherein,
Het1 and Het2 are independently selected from (i) a 5 to 7 membered heterocyclic group having 1-3 heteroatoms selected from O, N, and S, and (ii) a 6 to 10 membered bicyclic heterocyclic group having 1-3 heteroatoms selected from O, N, and S; wherein each of (i) or (ii) may by unsubstituted or substituted with 1 to 3 substituents selected from C₁-C₄ alkyl, NH₂, —O—R⁰, CN, and halo;
X and Z are independently selected from N and CH;
R¹ is selected from the group consisting of:
(i) C₁-C₁₀ alkyl, C₂-C₁₀ alkenyl, C₂-C₁₀ alkynyl, and C₃ to C₁₀ cycloalkyl; each of which is unsubstituted or is substituted by 1 to 3 substituents selected from the group consisting of halo, —N(R⁰)₂, CN, NO₂, —O—(C₂-C₁₀ alkyl), aryl, heteroaryl, and heterocyclyl;
(ii) aryl, heteroaryl and heterocyclyl; each of which is unsubstituted or is substituted by 1 to 3 substituents selected from the group consisting of halo, —N(R⁰)₂, CN, NO₂, C₁-C₆ alkyl, C₂-C₆ alkenyl, aryl, heteroaryl, heterocyclyl, —O—R⁰, —(C₁-C₁₀ alkyl)-OR⁰, —O—(C₁-C₁₀ alkyl)-OR⁰, —(C₁-C₁₀ alkyl)-N(R⁰)₂, —O—(C₂-C₁₀ alkyl)-N(R⁰)₂, —O—(C₂-C₁₀ alkyl)-C(═O)—N(R⁰)₂, —C(═O)—(C₂-C₁₀ alkyl)-N(R⁰)₂, —C(═O)N(R⁰)—(C₂-C₁₀ alkyl)-N(R⁰)₂, and —(C₁-C₆ alkyl)-aryl;
(iii) -aryl-C(═O)-aryl, -aryl-C(═O)-heteroaryl, and -aryl-C(═O)-heterocyclyl; each of which is unsubstituted or is substituted by 1 to 3 substituents selected from the group consisting of halo, —N(R⁰)₂, CN, NO₂, C₁-C₆ alkyl, C₂-C₆ alkenyl, and —O—R⁰;
(iv) -aryl-aryl, -aryl-heteroaryl, and -aryl-heterocyclyl; each of which is unsubstituted or is substituted by 1 to 3 substituents selected from the group consisting of halo, —N(R⁰)₂, CN, NO₂, C₁-C₆ alkyl, C₂-C₆ alkenyl, —O—R⁰; and
each R⁰ is independently selected from H and C₁-C₆ alkyl; or alternatively two adjacent R⁰ attached to a nitrogen atom may be taken together to form a 5 or 6-membered heterocyclic ring having 0 to 2 additional ring heteroatoms selected from N, O, and S, and which may be unsubstituted or substituted by 1 to 3 substituents selected form the group consisting of C₁-C₄ alkyl, NH₂, —OH, CN, and halo.

52. The pharmaceutical composition of claim 50, wherein the compound of Formula IX is Wyeth BMCL 20102644-13.

53. The pharmaceutical composition of claim 50, wherein the compound of Formula IX is Wyeth BMCL 20102648-27.

54. The pharmaceutical composition of claim 50, wherein the compound is an inhibitor of mTORC1.

55. The pharmaceutical composition of claim 50, wherein the compound is an inhibitor of mTORC2.

56. The pharmaceutical composition of claim 50, wherein the viral infection is by a herpesvirus.

57. The pharmaceutical composition of claim 50, wherein the viral infection is by a herpesvirus selected from the group consisting of herpes simplex virus type 1, herpes simplex virus type 2, varicella-zoster virus, human cytomegalovirus, Epstein-Barr virus, human herpesvirus 6 variant A, human herpesvirus 6 variant B, human herpesvirus 7, human herpesvirus 8, and cercopithecine herpesvirus 1.

58. The pharmaceutical composition of claim 50, further comprising administering to the mammalian subject an inhibitor of the unfolded protein response.

59. The pharmaceutical composition of claim 58, wherein the inhibitor of the unfolded protein response is 4-phenylbutyrate.

60. The pharmaceutical composition of claim 58, wherein the inhibitor of the unfolded protein response is tauroursodeoxycholic acid.

61-80. (canceled)

81. A method of treating or preventing viral infection in a mammal, comprising administering to a mammalian subject in need thereof a therapeutically effective amount of a compound or prodrug thereof, or pharmaceutically acceptable salt or ester of said compound or prodrug, wherein the compound is an inhibitor of a rapamycin-resistant function of mTOR and wherein the compound is a compound of Formula X: 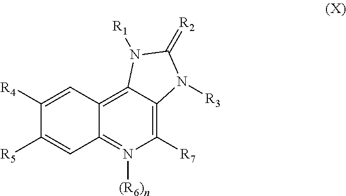
wherein
R₁ is selected from naphthyl or phenyl, which is unsubstituted or is substituted by one or two substituents independently selected from the group consisting of (i) halogen; (ii) lower alkyl unsubstituted or substituted by halogen, cyano, imidazolyl or triazolyl; (iii) cycloalkyl; (iv) amino substituted by one or two substituents independently selected from the group consisting of lower alkyl, lower alkyl sulfonyl, lower alkoxy and lower alkoxy lower alkylamino; (v) piperazinyl unsubstituted or substituted by one or two substituents independently selected from the group consisting of lower alkyl and lower alkyl sulfonyl; (vi) 2-oxo-pyrrolidinyl; (vii) lower alkoxy lower alkyl; (viii) imidazolyl; (ix) pyrazolyl; and (x) triazolyl;
R₂ is O or S;
R₃ is lower alkyl;
R₄ is selected from (i) pyridyl unsubstituted or substituted by halogen, cyano, lower alkyl, lower alkoxy or piperazinyl unsubstituted or substituted by lower alkyl; (ii) pyrimidinyl unsubstituted or substituted by lower alkoxy; (iii) quinolinyl unsubstituted or substituted by halogen; or (iv) quinoxalinyl;
R₅ is hydrogen or halogen;
n is 0 or 1;
R₆ is oxido; with the proviso that if n=1, the N-atom bearing the radical R₆ has a positive charge; and
R₇ is hydrogen or amino.

82. The method of claim 81, wherein the compound of Formula X is NVP-BEZ235.

83. The method of claim 81, wherein the compound is an inhibitor of mTORC1.

84. The method of claim 81, wherein the compound is an inhibitor of mTORC2.

85. The method of claim 81, wherein the viral infection is by a herpesvirus.

86. The method of claim 81, wherein the viral infection is by a herpesvirus selected from the group consisting of herpes simplex virus type 1, herpes simplex virus type 2, varicella-zoster virus, human cytomegalovirus, Epstein-Barr virus, human herpesvirus 6 variant A, human herpesvirus 6 variant B, human herpesvirus 7, human herpesvirus 8, and cercopithecine herpesvirus 1.

87. The method of claim 81, further comprising administering to the mammalian subject an inhibitor of the unfolded protein response.

88. The method of claim 87, wherein the inhibitor of the unfolded protein response is 4-phenylbutyrate.

89. The method of claim 87, wherein the inhibitor of the unfolded protein response is tauroursodeoxycholic acid.

90. A pharmaceutical composition for treatment or prevention of a viral infection comprising a therapeutically effective amount of a composition comprising (i) a compound or prodrug thereof, or pharmaceutically acceptable salt of said compound or prodrug; and (ii) a pharmaceutically acceptable carrier, wherein the compound is an inhibitor of a rapamycin-resistant function of mTOR and wherein the compound is a compound of Formula X: 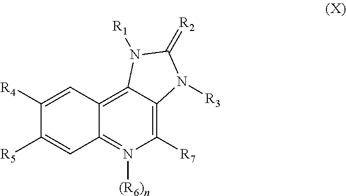
wherein
R₁ is selected from naphthyl or phenyl, which is unsubstituted or is substituted by one or two substituents independently selected from the group consisting of (i) halogen; (ii) lower alkyl unsubstituted or substituted by halogen, cyano, imidazolyl or triazolyl; (iii) cycloalkyl; (iv) amino substituted by one or two substituents independently selected from the group consisting of lower alkyl, lower alkyl sulfonyl, lower alkoxy and lower alkoxy lower alkylamino; (v) piperazinyl unsubstituted or substituted by one or two substituents independently selected from the group consisting of lower alkyl and lower alkyl sulfonyl; (vi) 2-oxo-pyrrolidinyl; (vii) lower alkoxy lower alkyl; (viii) imidazolyl; (ix) pyrazolyl; and (x) triazolyl;
R₂ is O or S;
R₃ is lower alkyl;
R₄ is selected from (i) pyridyl unsubstituted or substituted by halogen, cyano, lower alkyl, lower alkoxy or piperazinyl unsubstituted or substituted by lower alkyl; (ii) pyrimidinyl unsubstituted or substituted by lower alkoxy; (iii) quinolinyl unsubstituted or substituted by halogen; or (iv) quinoxalinyl;
R₅ is hydrogen or halogen;
n is 0 or 1;
R₆ is oxido; with the proviso that if n=1, the N-atom bearing the radical R₆ has a positive charge; and
R₇ is hydrogen or amino.

91. The pharmaceutical composition of claim 90, wherein the compound of Formula X is NVP-BEZ235.

92. The pharmaceutical composition of claim 90, wherein the compound is an inhibitor of mTORC1.

93. The pharmaceutical composition of claim 90, wherein the compound is an inhibitor of mTORC2.

94. The pharmaceutical composition of claim 90, wherein the viral infection is by a herpesvirus.

95. The pharmaceutical composition of claim 90, wherein the viral infection is by a herpesvirus selected from the group consisting of herpes simplex virus type 1, herpes simplex virus type 2, varicella-zoster virus, human cytomegalovirus, Epstein-Barr virus, human herpesvirus 6 variant A, human herpesvirus 6 variant B, human herpesvirus 7, human herpesvirus 8, and cercopithecine herpesvirus 1.

96. The pharmaceutical composition of claim 90, further comprising administering to the mammalian subject an inhibitor of the unfolded protein response.

97. The pharmaceutical composition of claim 96, wherein the inhibitor of the unfolded protein response is 4-phenylbutyrate.

98. The pharmaceutical composition of claim 96, wherein the inhibitor of the unfolded protein response is tauroursodeoxycholic acid.

99-116. (canceled)

117. A method of treating or preventing viral infection in a mammal, comprising administering to a mammalian subject in need thereof a therapeutically effective amount of a compound or prodrug thereof, or pharmaceutically acceptable salt or ester of said compound or prodrug, wherein the compound is an inhibitor of a rapamycin-resistant function of mTOR and wherein the compound is selected from AZD2014, CC-223 (TORKi), SB-2015, NVP-BGT-226, LY294002, GSK2126458, XL147, XL765, PF-04691502, P-2281, GDC-0980, ETP-45658, WJD008, PKI-179, PKI-402, PKI-587, OXA-01, Compound 401, ZSTK-474, PI-103, and Palomid-529.

118. The method of claim 117, wherein the viral infection is by a herpesvirus.

119. The method of claim 117, wherein the viral infection is by a herpesvirus selected from the group consisting of herpes simplex virus type 1, herpes simplex virus type 2, varicella-zoster virus, human cytomegalovirus, Epstein-Barr virus, human herpesvirus 6 variant A, human herpesvirus 6 variant B, human herpesvirus 7, human herpesvirus 8, and cercopithecine herpesvirus 1.

120. A pharmaceutical composition for treatment or prevention of a viral infection comprising a therapeutically effective amount of a composition comprising (i) a compound or prodrug thereof, or pharmaceutically acceptable salt of said compound or prodrug; and (ii) a pharmaceutically acceptable carrier, wherein the compound is an inhibitor of a rapamycin-resistant function of mTOR and wherein the compound is selected from AZD2014, CC-223 (TORKi), SB-2015, NVP-BGT-226, LY294002, GSK2126458, XL147, XL765, PF-04691502, P-2281, GDC-0980, ETP-45658, WJD008, PKI-179, PKI-402, PKI-587, OXA-01, Compound 401, ZSTK-474, PI-103, and Palomid-529.

121. The pharmaceutical composition of claim 120, wherein the viral infection is by a herpesvirus.

122. The pharmaceutical composition of claim 120, wherein the viral infection is by a herpesvirus selected from the group consisting of herpes simplex virus type 1, herpes simplex virus type 2, varicella-zoster virus, human cytomegalovirus, Epstein-Barr virus, human herpesvirus 6 variant A, human herpesvirus 6 variant B, human herpesvirus 7, human herpesvirus 8, and cercopithecine herpesvirus 1.

123-128. (canceled)


SEQUENCE LISTING (7 sequences)

SEQ ID NO: 1 - DNA, 21 bp, Artificial
  siRNA
aaacgcaucuccgaguuggac

SEQ ID NO: 2 - DNA, 22 bp, Artificial
  siRNA
gcaagcugacccugaaguucau

SEQ ID NO: 3 - DNA, 19 bp, Artificial
  siRNA
gaguuacagucgggcauau

SEQ ID NO: 4 - DNA, 19 bp, Artificial
  synthetic primer
gtgtcccattcccgactcg

SEQ ID NO: 5 - DNA, 19 bp, Artificial
  synthetic primer
ttcacaacgtccacccacc

SEQ ID NO: 6 - DNA, 21 bp, Artificial
  synthetic primer
tcctcctgagcgcaagtactc

SEQ ID NO: 7 - DNA, 22 bp, Artificial
  synthetic primer
cggactcgtcatactcctgctt